(19) United States
(12) Patent Application Publication
(10) Pub. No.: US 2009/0004641 A1
(43) Pub. Date: Jan. 1, 2009

(54) SITE-SPECIFIC LABELING OF AFFINITY PEPTIDES IN FUSION PROTEINS

(75) Inventors: Kyle Richard Gee, Springfield, OR (US); Courtenay Rae Hart, Eugene, OR (US); Richard Haugland, Olympia, WA (US); Wayne Forrest Patton, Newton, MA (US); Scott Whitney, San Diego, CA (US)

Correspondence Address: INVITROGEN CORPORATION;C/O INTELLEVATE, P.O. BOX 52050, MINNEAPOLIS, MN, 55402, US

(73) Assignee: INVITROGEN CORPORATION, Carlsbad, CA (US)

(21) Appl. No.: 11/871,031

(22) Filed: Oct. 11, 2007

Related U.S. Application Data
(63) Continuation of application No. 10/966,536, filed on Oct. 14, 2004, now abandoned.
Application No. 10/966,536 is a continuation-in-part of application No. 10/661,451, filed on Sep. 12, 2003.
(60) Provisional application No. 60/511,252, filed on Oct. 14, 2003.
(60) Provisional application No. 60/410,612, filed on Sep. 12, 2002.
(60) Provisional application No. 60/458,472, filed on Mar. 28, 2003.

(51) Int. Cl. C12Q 1/00; C07D 401/14; C07D 207/30

(52) U.S. Cl. 435/4; 546/256; 548/518

(57) ABSTRACT

The present invention provides methods and fluorescent compounds that facilitate detecting and labeling of a fusion protein by being capable of selectively binding to an affinity tag. The fluorescent compounds have the general formula A(B)n, wherein A is a fluorophore, B is a binding domain that is a charged chemical moiety, a protein or fragment thereof and n is an integer from 1-6 with the proviso that the protein or fragment thereof not be an antibody or generated from an antibody. The present invention provides specific fluorescent compounds and methods used to detect and label fusion proteins that contain a poly-histidine affinity tag. These compounds have the general formula A(L)m(B)n wherein A is a fluorophore, L is a linker, B is an acetic acid binding domain, m is an integer from 1 to 4 and n is an integer from 1 to 6. The acetic acid groups interact directly with the positively charged histidine residues of the affinity tag to effectively label and detect a fusion protein containing such an affinity tag when present in an acidic or neutral environment.

### CROSS-REFERENCE TO RELATED APPLICATIONS

This application claims priority to U.S. Ser. No. 60/511,252, filed Oct. 14, 2003, which disclosure is herein incorporated by reference. This application is a continuation-in-part of U.S. Ser. No. 10/661,451, filed Sep. 12, 2003, which claims priority to U.S. Ser. No. 60/410,612, filed Sep. 12, 2002; and U.S. Ser. No. 60/458,472, filed Mar. 28, 2002, which disclosures are herein incorporated by reference.

### FIELD OF THE INVENTION

The present invention relates to novel compositions and methods for the detection and isolation of fusion proteins comprising affinity tag sequences. The invention has applications in the fields of molecular biology and proteomics.

### BACKGROUND OF THE INVENTION

The present invention relates to fluorescent compounds that have selective affinity, and bind with specificity to affinity tag-containing fusion proteins. Such compounds being particularly useful for the detection, site-specifically labeling and monitoring of desired recombinant fusion proteins.

Typically, recombinant fusion proteins comprise a synthetic leader peptide or protein fragment linked to independently derived polypeptides. In 1965 it was demonstrated that an amino acid sequence not normally part of a given operon can be inserted within the operon and be controlled by the operon (Jacob, F. et al. (1965) J. Mol. Biol. 13, 704). Therefore, the leader sequence of recombinant fusion proteins can facilitate protein expression, detection and purification by providing, for example, enzymatic activity enabling identification of fusion proteins, an amino acid sequence recognized by cellular secretory mechanism, or a sequence having distinctive chemical or antigenic characteristics useful in purifying and detection of the fusion protein by ion exchange, reverse phase, immunoaffinity and affinity chromatographic media. In general, polyanionic peptides and polycationic peptides bind to ion-exchangers, hydrophobic peptides bind to reverse-phase media and peptides that are immunogenic can be bound by antibodies.

Immobilized metal-ion affinity chromatography (IMAC) relies upon the interaction of exposed histidine and cysteine residues on proteins with certain transition metals, such as Ni²⁺, Co²⁺, Zn²⁺, Cu²⁺ and Fe³⁺ (Porath, J., et al. (1975) Nature 258:598-599; Winzerling, J., et al. (1992) Methods 4:4-13; Yip, T. and Hutchens, T. (1994) Molecular Biotechnol. 1:151-164). Protein interaction with immobilized metal ions is a selective and versatile, high-affinity adsorption procedure. The basic principles of IMAC are commonly exploited to facilitate the purification of recombinant proteins.

The poly-histidine affinity tag is a transition metal-binding peptide sequence comprising a string of four to ten histidine residues. When a DNA sequence corresponding to the poly-histidine affinity tag is fused in frame with a gene, the resulting fusion protein can readily be purified by IMAC using a nickel- or cobalt-charged resin. Though a variety of fusion affinity tags have been developed over the years, the poly-histidine affinity tag is popular because it requires minimal addition of extra amino acids to the recombinant protein, rarely interferes with protein folding, is poorly immunogenic and allows for rapid purification of the target protein by IMAC.

Unfortunately, the detection of poly-histidine affinity tag containing fusion proteins after electrophoresis usually requires multiple time-consuming steps, including transfer of the gel to a membrane, blocking of unoccupied sites on the membrane with protein or detergent solutions, incubation with a poly-histidine affinity tag-binding agent (primary antibody, biotin-nitrilotriacetic acid or HRP-nitrilotriacetic acid), incubation with a secondary detection agent (antibody-reporter enzyme conjugate, streptavidin-reporter enzyme conjugate), and incubation with a visualization reagent (colorimetric, fluorogenic or chemiluminescent reagent). Specifically, biotinylated nitrilotriacetic acid (NTA) has been used in combination with streptavidin-horseradish peroxidase or streptavidin-alkaline phosphatase conjugates and chemiluminescent or colorimetric substrates in order to detect poly-histidine affinity tag containing fusion proteins after electroblotting (Hochuli, E. and Piesecki, S. (1992) Methods 4: 68-72; O'Shannessy, D., et al. (1995) Anal. Biochem. 229:119-124; McMahan, S. and Burgess, R. (1996) Anal Biochem. 236: 101-106). In addition, direct reporter enzyme-nitrilotriacetate-nickel conjugates have been employed for detection of poly-histidine affinity tag containing fusion proteins on electroblots (Botting, C. and Randall, R. (1995) BioTechniques 19: 362-363; Jin, L., et al. (1995) Anal. Biochem. 229: 54-60). Similarly, colloidal gold with nitrilotriacetic acid conjugated to its surface has been employed to detect poly-histidine affinity tag containing fusion proteins on blots after a silver enhancement step (Hainfeld, J., et al. (1999) J. Struct. Biol. 127: 185-198). Finally, though poly-histidine affinity tag is not particularly immunogenic, a number of high affinity monoclonal antibodies specific to the peptide have been generated to detect affinity tag containing fusion proteins by standard electroblotting methods (Zentgraf, H., et al. (1995) Nucleic Acids Res. 23: 3347-3348; Pogge von Strandmann, E., et al. (1995) Protein Eng. 8: 733-735; Lindner, P., et al. (1997) BioTechniques 22: 140-149).

Examples of immunogenic affinity tags include protein A, c-myc (Roth et al, (1991) J. Cell Biol. 115:587-596), myc (EQKLISEEDL; Evan G I, et al. (1985) Mol. Cell. Biol. 5:3610-3616; Munro S. and Pelham HRB, (1987) Cell 48:899-907; Borjigin J. and Nathans J., (1994) 269:14715-14727; Smith D J, (1997) BioTechniques 23:116-120) FLAG® (Hopp T. P. et al. (1988) Biotechnology 6:1204; Prickett, K. S. et al. (1989) BioTechniques 7:580-589; Gerard N P and Gerard C, (1990) Biochemistry 29:9274-9281; Einhauer A. and Jungbauer A. (2001) J. Biochem Biophys. Methods 49:455-465; U.S. Pat. Nos. 4,703,004; 4,851,341 and 5,011,912), GST (Glutathione-S-transferase), HA, derived from the influenza hemagglutinin protein (Wilson I A, et al., (1984) Cell, 37:767; Field J. et al. Mol. Cell Biol. (1988) 8:2159-2165; Xu Y, et al. (2000) Mol Cell Biol. 20:2138-2146), IRS (RYIRS; Liang T C et al. (1996) 329:208-214; Luo W et al (1996) Arch. Biochem. Biophys. 329:215-220), AU1 and AU5 (DTYRYI and TDFLYK; Lim P S et al. (1990) J. Infect. Dis. 162:1263-1269; Goldstein D J et al. (1992) 190:889-893; Koralnik I J et al. (1993) J. Virol. 67:2360-2366), glu-glu (a 9 amino acid epitope from polyoma virus medium T antigen, EEEEYMPME; Grussenmeyer, T. et al. (1985) PNAS. USA 82:7952-7954; Rubinfeld. B. et al. (1991) Cell 65:1033-1042), KT3 (an 11 amino acid epitope from the SV40 large T antigen, KPPTPPPEPET; MacArthur H. and Walter G. (1984) J, Virol. 52:483-491; Martin G A et al. (1990) 63:843-849; Di Paolo G et al. (1997) 272:5175-5182), T7 (an 11 amino acid leader peptide from T7 major capsid protein), S-TAG, HSV (an 11 amino acid peptide from herpes simplex virus glycoprotein D), VSV-G (an 11 amino acid epitope from the carboxy terminus of vesicular stomatitis virus glycoprotein, YTDIEMNRLGK; Kreis T. (1986) EMBO J. 5:931-941; Turner J R et al (1996) 271:7738-7744), Anti-Xpress (8 amino acid epitope, DLYDDDK), and VS (14 amino acid epitope from paramoxyvirus SV5, GKPIPNPLLGLDST).

Typically, immunogenic affinity tags are detected with labeled antibodies wherein the label can be an enzyme, fluorophore, hapten or any label known to one skilled in the art and the antibodies, directly or indirectly, detect the affinity containing fusion protein. Immunogenic affinity tags can also be detected in a multistep assay using ruthenium labeled anti-affinity tag antibodies that produce electrochemiluminescence (ECL) (ORIGEN®, U.S. Pat. Nos. 5,310,687; 5,714,089; 5,453,356; 6,140,138; 5,804,400 and 5,238,808) indicating the presence of the affinity tag. Electrochemiluminescence is the process by which light generation occurs when a low voltage is applied to an electrode, triggering a cyclical oxidation and reduction reaction of a ruthenium metal ion bound to the compound to be detected. The ruthenium labeled antibody is captured on a solid surface by the affinity tag, a second oxidation reaction component, tripropylamine (TPA), is introduced into the cell and a voltage is applied. The TPA reduces the ruthenium, which receives the electron in an excited state and then decays to the ground state releasing a photon in the process.

The FLAG® affinity tag was designed in conjunction with antibodies for the purpose of detection and purification of fusion proteins (Hopp T. P. et al. (1988) Biotechnology 6:1204; Prickett, K. S. et al. (1989) BioTechniques 7:580-589, supra). As such, the use of anti-FLAG® antibodies are widely used to detect and purify FLAG® affinity tag containing fusion proteins. The FLAG® sequence typically consists of DYKDDDDK, D=Asp, Y=Tyr and K=Lys, but any combination of 3 to 6 aspartic or glutamic acid residues is also considered a FLAG® sequence. The sequence is hydrophilic and highly immunogenic. The FLAG® affinity tag has effectively been used in various expression systems for the detection and purification of recombinant fusion proteins (Brizzard et al. (1994) BioTechniques 16:730-735; Lee et al. (1994) Nature 372:739-746; Xu et al. (1993) Development 117:1223-1237; Dent et al. (1995) Mol. Cell Biol. 15:4125-4135; Ritchie et al. (1999) BioChem Journal 338:305-10.) Recently, the FLAG® affinity tag was used to detect fusion proteins wherein the use of antibodies was not employed (Buranda T. et al. (2001) Anal. Biochemistry 298:151-162). The FLAG® sequence was synthesized with fluorescein and/or biotin as a label and tag, respectively, wherein the peptides were bound to streptavidin beads and the fluorescein was detected using flow cytometry.

While antibodies against GST are available for both purification and detection (Molecular Probes, Inc., Eugene, Oreg.) the affinity tag is typically purified using glutathione resin (U.S. Pat. Nos. 5,654,176; 6,303,128 and 6,013,462). Glutathione is a ubiquitous tripeptide that binds with high affinity to the GST enzyme.

An affinity tag that is not generally immunogenic and does not readily bind metal ions or chemical moieties includes calmodulin-binding peptides (U.S. Pat. Nos. 5,585,475; 6,316,409 and 6,117,976). These affinity tags are routinely purified using columns wherein beads are covalently attached to calmodulin. In the presence of calcium the calmodulin protein binds the calmodulin affinity tag with high affinity because calcium induces a conformational change in calmodulin increasing the affinity of the protein for the affinity tag. Calmodulin affinity tags are advantageous in certain applications because the captured fusion protein can be eluted from a column using a metal chelating moiety instead of harsh denaturing conditions.

Another affinity tag that is not generally immunogenic includes the binding site for the FlAsH reagent, CCXXCC wherein X is an amino acid other than cysteine (Griffin et al (2000) Methods in Enzymology 327:565-578; Griffin et al (1998) Science 281:269-272; Thorn et al (2000) Protein Science 9:213-217). The FlAsH reagent is a fluorescein molecule that has been substituted by two arsenical groups such that the reagent interacts with the α-helical structure of the CCXXCC sequence (Adams et al (2002) Journal of American Chemical Society 124: 6063-6076). For binding to occur the thiols of the cysteine residues must not be disulfide bonded or chelated by a metal ion. Thus, the FlAsH reagent is typically used to label proteins in vivo due to these limitations for in vitro labeling. Therefore a reducing agent must be used for binding to occur and a buffer must be free of metal ions.

There is a need in the art for a staining reagent that has low non-specific binding, is readily visualized on a transluminator with a standard video camera, and has similar sensitivity on Bis-Tris, Tris-Glycine, Tris-Acetate and other gels, including pH neutral gels, with picomolar detection limits of histidine-labeled proteins.

The fluorescent compounds and methods of the present invention have been developed for the fluorescence detection of affinity tag containing fusion proteins directly in polymeric gels (with or without sodium dodecyl sulfate (SDS)), without the requirement for electroblotting, blocking, reporter enzymes or secondary detection reagents. These present fluorescent compounds are advantages over FlAsH wherein a reducing agent is not required and a metal ion may be present in the buffer solution or pre-complexed to the fluorescent compound. These compounds take advantage of the charged residues of the affinity tag wherein the binding domains of the present invention are covalently attached to a fluorophore for selective detection of a wide range of affinity tag containing fusion proteins. These compounds and methods of the present invention provide a significant improvement over the prior art for detecting, monitoring and quantitating affinity tag containing fusion proteins.

### SUMMARY OF THE INVENTION

The present invention provides methods and fluorescent compounds that specifically and selectively bind to affinity tags of fusion proteins. The compounds of the present invention facilitate detecting and labeling of a fusion protein by being capable of selectively binding to an affinity tag. The methods for detecting a fusion protein containing an affinity tag comprises contacting a sample with a staining solution and then illuminating the sample whereby the fusion protein is detected. The staining solution comprises a fluorescent compound and a buffer wherein the buffer optionally comprises a metal ion. The fluorescent compounds, as used herein, are defined as a compound that is capable of selectively binding, directly or indirectly to an affinity tag. In one embodiment the fluorescent compound is pre-loaded with metal ions.

These reagents are useful in many applications including, by way of non-limiting example, detection of proteins that have been separated in polyacrylamide gels by SDS-page electrophoresis. The new staining reagents have faster kinetics and can tolerate some SDS in the solution allowing for staining to be complete in a faster time period. In one embodiment, the staining is complete in about 2 hours. One version of the staining reagent developed uses a rhodamine fluorophore. The use of rhodamine and other fluorophores as described herein allows the stain to be used with standard laboratory equipment, such as ethidium bromide filtered cameras using transillumination, as well as more specialized laser based systems which have optics designed for use with Cy3 or other dyes.

The fluorescent compounds have the general formula A(B)n, wherein A is a fluorophore, B is a binding domain that is a charged chemical moiety, a protein or fragment thereof and n is an integer from 1-6 with the proviso that the protein or fragment thereof not be an antibody or generated from an antibody. The binding domain of the fluorescent compound may bind directly or indirectly to the affinity tag. When the fluorescent compound binds directly, the charged chemical moiety or protein of the binding domain interacts directly to form a non-covalent bond between the fluorescent compound and the affinity tag of the fusion protein. When the compounds of the present invention bind indirectly, a metal ion facilitates the indirect binding by having affinity for both the charged amino acid residues of the affinity tag and the binding domain of the fluorescent compound. The indirect binding of the fluorescent compound results in a ternary complex of the fluorescent compound, metal ion and affinity tag of the fusion protein. The metal ion may be present in the staining solution. Typically, the metal ion, if present, is pre-complexed with the fluorescent compound. In this instance, the staining solution typically has a pH about 7.0 to about 9.0 and contains buffering components that maintain the neutral to slightly basic pH. Such buffering components, include but are not limited to, phosphate, Tris and tricine.

The present invention provides specific fluorescent compounds and methods used to detect and label fusion proteins that contain a poly-histidine affinity tag or a poly-arginine affinity tag. These compounds have the general formula A(L)m(B)n wherein A is a fluorophore, L is a linker, B is a binding domain, m is an integer from 1 to 4 and n is an integer from 1 to 6. The linker functions to covalently attach the fluorophore to the binding domain wherein the resulting fluorescent compound contains an acetic acid binding domain. The acetic acid groups interact directly with the positively charged histidine or arginine residues of the affinity tag to effectively label and detect a fusion protein containing such an affinity tag when present in a slightly acidic or neutral environment. Alternatively, the acetic acid groups of the fluorescent compound have an affinity for the metal ions nickel and cobalt wherein the metal ions also have affinity for the poly-histidine affinity tag of the fusion peptide. In this instance it is preferred that the fluorescent compounds be pre-loaded with the metal ions and are present in a staining solution that has a neutral to slight basic pH, with a pH about 7.0 to about 9.0. Exemplary compounds include 17a and 19. This indirect labeling and detection of the fusion protein may in certain circumstances be as effective as the direct method that does not utilize the metal ions for labeling and detecting fusion proteins containing poly-histidine affinity tags.

Thus, in one embodiment, the present staining solution comprises
a) a fluorescent compound having formula A(L)m(B)n wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH of about 7.0 to about 9.0 with the proviso that the binding domain does not comprise an antibody or fragment thereof.

Fluorophore of the present fluorescent compounds include, but are not limited to, xanthene, coumarin, cyanine, acridine, anthracene, benzofuran, indole or borapolyazaindacene. The present binding domains include, but are not limited to, NTA or BAPTA.

The present invention also provides kits that comprise a present staining solution. The kits optionally further comprise a molecular weight markers, a fixing solution, a wash solution or an additional detection reagent. Additional detection reagents include, but are not limited to, total protein stains.

### BRIEF DESCRIPTION OF THE FIGURES

FIG. 1: Shows the detection of a poly-histidine affinity tag containing fusion protein (urate oxidase) labeled with Compound 2 in a staining solution containing (1A) nickel ions and (2B) without nickel ions. In this particular assay, Compound 2 demonstrates an increased sensitivity for the poly-histidine affinity tag in the absence of nickel ions.

FIG. 2: Shows the detection of a (2A) poly-histidine affinity tag containing fusion protein (Oligomycin sensitivity conferring protein; OSCP) labeled with Compound 2 in a staining solution containing nickel ions followed by the detection of (2B) total protein using the total protein stain SYPRO® Ruby. This assay demonstrates that Compound 2 is selective for the poly-histidine affinity tag.

FIG. 3: Shows the detection of a (3A) poly-histidine affinity tag containing fusion protein (Oligomycin sensitivity conferring protein; OSCP) labeled with Compound 15 in a staining solution containing nickel ions followed by the detection of (3B) total protein using the total protein stain SYPRO® Ruby. This assay demonstrates that Compound 15 is selective for the poly-histidine affinity tag.

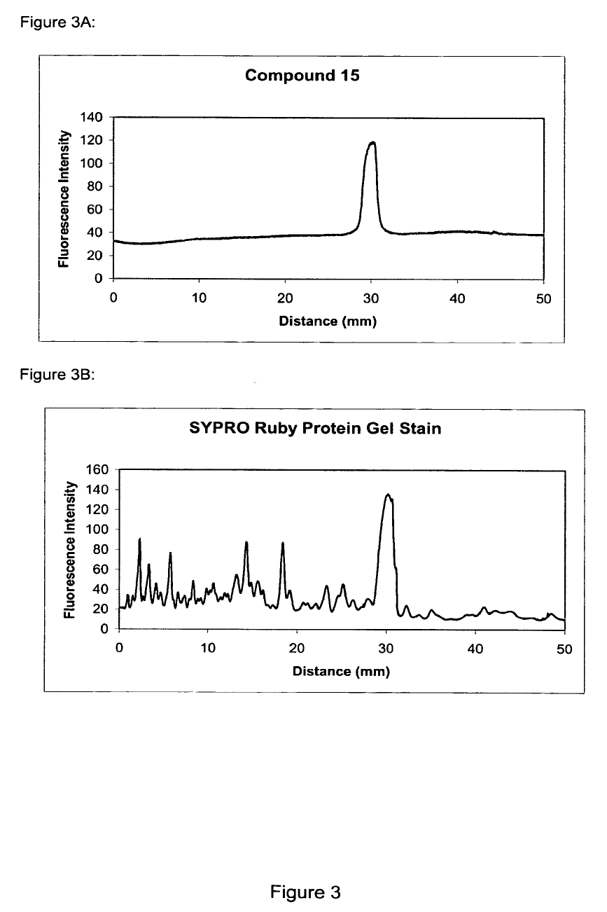

FIG. 4: Shows the detection of GST affinity tag using Texas Red X-glutathione fluorescent compound on a polyacrylamide gel. Purified glutathione S-transferase (1 μg) at 24 and 25 mm from the gel origin (2 peaks) stained with 5 μM Texas Red X-Glutathione in 50 mM PIPES pH 6.5. Imaged on the Fuji FLA3000 at 532 nm excitation, 580LP filter. See, Example 20.

FIG. 5: Shows detection of poly-histidine affinity tag containing fusion proteins using Compound 19 in NuPAGE gels with a laser-based scanner.

FIG. 6: Shows detection of poly-histidine affinity tag containing fusion proteins using Compound 19 in NuPAGE Bis-Tris gel using microwave assisted staining method.

### DETAILED DESCRIPTION OF THE INVENTION

### Introduction

In accordance with the present invention, methods and compositions are provided that label and detect fusion proteins by specifically and selectively binding to an affinity tag of a fusion protein. The affinity tag is defined to include any affinity tag known to one skilled in the art and fused to a protein of interest for the purposes of detection and purification. The fluorescent compound is defined as being capable of binding to an affinity tag and includes the general formula A(B)n wherein A is any fluorophore known to one skilled in the art, B is a selected binding domain of the present invention and n is an integer from 1 to 6. The binding domain is a chemical moiety, protein or fragment thereof with the proviso that the fluorescent compound does not comprise an antibody or fragment thereof. The binding domain may interact directly, selectively binding to the affinity tag, or indirectly, wherein a third component forms a ternary complex between the fluorescent compound and the affinity tag. Typically, the third component is a metal ion wherein the metal ion has affinity for both the affinity tag and the binding domain. Alternatively, the third component does not have an affinity for the affinity tag but induces a conformational change to the binding domain such that the binding domain has an affinity for an affinity tag. The binding moiety can be a charged chemical moiety such as a metal chelating group, a protein, a peptide or fragment thereof such as calmodulin, provided that the binding domain is not an antibody or generated from an antibody. Thus, the present invention contemplates a wide range of fluorescent compounds that can be used to detect a myriad of affinity tags that are fused to a protein of interest whereby detection of a fusion protein is determined by a fluorescent signal generated from the fluorescent compound.

In addition to the components of the fluorescent compound that confer selectivity for an affinity tag, the staining solution also plays a critical role in determining selectivity and is typically altered depending on the affinity tag and the assay method. The staining solution contains a buffer and a fluorescent compound wherein the buffering components fine-tune the selectivity of the fluorescent compound for an affinity tag. For example, we have found in one embodiment that for the selective detection of poly-histidine containing fusion proteins on a gel that the buffer is preferably slightly acidic or neutral, contains a salt and has a pKa of about 6.0 to about 7.05. It appears that a pKa value of the buffer that is similar to the pKa value of the imidazole ring of histidine, which is about 7.05, results in a buffer that facilitates the non-covalent binding of the fluorescent compound to the poly-histidine affinity tag. Thus, in one aspect preferred buffers for the detection of poly-histidine affinity tag containing fusion proteins includes, but are not limited to, Good's buffer, PIPES and MOPS buffers.

Alternatively, in another embodiment we have found that when the compound of the present invention is pre-complexed with a metal ion, such as nickel, that the buffer be neutral or slightly basic to facilitate binding to poly-histidine fusion proteins. A preferred buffer component includes, but is not limited to, phosphate.

### DEFINITIONS

Before describing the present invention in detail, it is to be understood that this invention is not limited to specific compositions or process steps, as such may vary. It must be noted that, as used in this specification and the appended claims, the singular form “a”, “an” and “the” include plural referents unless the context clearly dictates otherwise. Thus, for example, reference to “a fusion protein” includes a plurality of proteins and reference to “a fluorescent compound” includes a plurality of compounds and the like.

Unless defined otherwise, all technical and scientific terms used herein have the same meaning as commonly understood by one of ordinary skill in the art to which this invention is related. The following terms are defined for purposes of the invention as described herein.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are encompassed within the scope of the present invention.

The compounds of the invention may be prepared as a single isomer (e.g., enantiomer, cis-trans, positional, diastereomer) or as a mixture of isomers. In a preferred embodiment, the compounds are prepared as substantially a single isomer. Methods of preparing substantially isomerically pure compounds are known in the art. For example, enantiomerically enriched mixtures and pure enantiomeric compounds can be prepared by using synthetic intermediates that are enantiomerically pure in combination with reactions that either leave the stereochemistry at a chiral center unchanged or result in its complete inversion. Alternatively, the final product or intermediates along the synthetic route can be resolved into a single stereoisomer. Techniques for inverting or leaving unchanged a particular stereocenter, and those for resolving mixtures of stereoisomers are well known in the art and it is well within the ability of one of skill in the art to choose and appropriate method for a particular situation. See, generally, Furniss et al. (eds.), VOGEL'S ENCYCLOPEDIA OF PRACTICAL ORGANIC CHEMISTRY 5^{TH} ED., Longman Scientific and Technical Ltd., Essex, 1991, pp. 809-816; and Heller, Acc. Chem. Res. 23: 128 (1990).

Although typically not shown for the sake of clarity, any overall positive or negative charges possessed by any of the compounds of the invention are balanced by a necessary counterion or counterions. Where the compound of the invention is positively charged, the counterion is typically selected from, but not limited to, chloride, bromide, iodide, sulfate, alkanesulfonate, arylsulfonate, phosphate, perchlorate, tetrafluoroborate, tetraarylborate, nitrate, hexafluorophosphate, and anions of aromatic or aliphatic carboxylic acids. Where the compound of the invention is negatively charged, the counterion is typically selected from, but not limited to, alkali metal ions, alkaline earth metal ions, transition metal ions, ammonium or substituted ammonium ions. Preferably, any necessary counterion is biologically compatible, is not toxic as used, and does not have a substantially deleterious effect on biomolecules. Counterions are readily changed by methods well known in the art, such as ion-exchange chromatography, or selective precipitation.

The compounds of the present invention may also contain unnatural proportions of atomic isotopes at one or more of the atoms that constitute such compounds. For example, the compounds may be radiolabeled with radioactive isotopes, such as for example tritium (3H), iodine-125 (¹²⁵I) or carbon-14 (¹⁴C). All isotopic variations of the compounds of the present invention, whether radioactive or not, are intended to be encompassed within the scope of the present invention.

Where substituent groups are specified by their conventional chemical formulae, written from left to right, they equally encompass the chemically identical substituents, which would result from writing the structure from right to left, e.g., —CH₂O— is intended to also recite —OCH₂—.

The term “acyl” or “alkanoyl” by itself or in combination with another term, means, unless otherwise stated, a stable straight or branched chain, or cyclic hydrocarbon radical, or combinations thereof, consisting of the stated number of carbon atoms and an acyl radical on at least one terminus of the alkane radical. The “acyl radical” is the group derived from a carboxylic acid by removing the —OH moiety therefrom.

The term “alkyl,” by itself or as part of another substituent means, unless otherwise stated, a straight or branched chain, or cyclic hydrocarbon radical, or combination thereof, which may be fully saturated, mono- or polyunsaturated and can include divalent (“alkylene”) and multivalent radicals, having the number of carbon atoms designated (i.e. C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, cyclohexyl, (cyclohexyl)methyl, cyclopropylmethyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. The term “alkyl,” unless otherwise noted, is also meant to include those derivatives of alkyl defined in more detail below, such as “heteroalkyl.” Alkyl groups that are limited to hydrocarbon groups are termed “homoalkyl”.

Exemplary alkyl groups of use in the present invention contain between about one and about twenty-five carbon atoms (e.g. methyl, ethyl and the like). Straight, branched or cyclic hydrocarbon chains having eight or fewer carbon atoms will also be referred to herein as “lower alkyl”. In addition, the term “alkyl” as used herein further includes one or more substitutions at one or more carbon atoms of the hydrocarbon chain fragment.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a straight or branched chain, or cyclic carbon-containing radical, or combinations thereof, consisting of the stated number of carbon atoms and at least one heteroatom selected from the group consisting of O, N, Si, P and S, and wherein the nitrogen, phosphorous and sulfur atoms are optionally oxidized, and the nitrogen heteroatom is optionally be quaternized, and the sulfur atoms are optionally trivalent with alkyl or heteroalkyl substituents. The heteroatom(s) O, N, P, S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, and —CH═CH—N(CH₃)—CH₃. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like.

The term “aryl” means, unless otherwise stated, a polyunsaturated, aromatic moiety that can be a single ring or multiple rings (preferably from 1 to 4 rings), which are fused together or linked covalently. Specific examples of aryl substituents include, but are not limited to, substituted or unsubstituted derivatives of phenyl, biphenyl, o, m-, or p-terphenyl, 1-naphthyl, 2-naphthyl, 1-, 2-, or 9-anthryl, 1-, 2-, 3-, 4-, or 9-phenanthrenyl and 1-, 2- or 4-pyrenyl. Preferred aryl substituents are phenyl, substituted phenyl, naphthyl or substituted naphthyl.

The term “heteroaryl” as used herein refers to an aryl group as defined above in which one or more carbon atoms have been replaced by a non-carbon atom, especially nitrogen, oxygen, or sulfur. For example, but not as a limitation, such groups include furyl, tetrahydrofuryl, pyrrolyl, pyrrolidinyl, thienyl, tetrahydrothienyl, oxazolyl, isoxazolyl, triazolyl, thiazolyl, isothiazolyl, pyrazolyl, pyrazolidinyl, oxadiazolyl, thiadiazolyl, imidazolyl, imidazolinyl, pyridyl, pyridaziyl, triazinyl, piperidinyl, morpholinyl, thiomorpholinyl, pyrazinyl, piperainyl, pyrimidinyl, naphthyridinyl, benzofuranyl, benzothienyl, indolyl, indolinyl, indolizinyl, indazolyl, quinolizinyl, qunolinyl, isoquinolinyl, cinnolinyl, phthalazinyl, quinazolinyl, quinoxalinyl, pteridinyl, quinuclidinyl, carbazolyl, acridinyl, phenazinyl, phenothizinyl, phenoxazinyl, purinyl, benzimidazolyl and benzthiazolyl and their aromatic ring-fused analogs. Many fluorophores are comprised of heteroaryl groups and include, without limitations, xanthenes, oxazines, benzazolium derivatives (including cyanines and carbocyanines), borapolyazaindacenes, benzofurans, indoles and quinazolones.

Where a ring substituent is a heteroaryl substituent, it is defined as a 5- or 6-membered heteroaromatic ring that is optionally fused to an additional six-membered aromatic ring(s), or is fused to one 5- or 6-membered heteroaromatic ring. The heteroaromatic rings contain at least 1 and as many as 3 heteroatoms that are selected from the group consisting of O, N or S in any combination. The heteroaryl substituent is bound by a single bond, and is optionally substituted as defined below.

Specific examples of heteroaryl moieties include, but are not limited to, substituted or unsubstituted derivatives of 2- or 3-furanyl; 2- or 3-thienyl; N—, 2- or 3-pyrrolyl; 2- or 3-benzofuranyl; 2- or 3-benzothienyl; N—, 2- or 3-indolyl; 2-, 3- or 4-pyridyl; 2-, 3- or 4-quinolyl; 1-, 3-, or 4-isoquinolyl; 2-, 4-, or 5-(1,3-oxazolyl); 2-benzoxazolyl; 2-, 4-, or 5-(1,3-thiazolyl); 2-benzothiazolyl; 3-, 4-, or 5-isoxazolyl; N—, 2-, or 4-imidazolyl; N—, or 2-benzimidazolyl; 1- or 2-naphthofuranyl; 1- or 2-naphthothienyl; N—, 2- or 3-benzindolyl; 2-, 3-, or 4-benzoquinolyl; 1-, 2-, 3-, or 4-acridinyl. Preferred heteroaryl substituents include substituted or unsubstituted 4-pyridyl, 2-thienyl, 2-pyrrolyl, 2-indolyl, 2-oxazolyl, 2-benzothiazolyl or 2-benzoxazolyl.

The above heterocyclic groups may further include one or more substituents at one or more carbon and/or non-carbon atoms of the heteroaryl group, e.g., alkyl; aryl; heterocycle; halogen; nitro; cyano; hydroxyl, alkoxyl or aryloxyl; thio or mercapto, alkyl- or arylthio; amino, alkyl-, aryl-, dialkyl-, diaryl-, or arylalkylamino; aminocarbonyl, alkylaminocarbonyl, arylaminocarbonyl, dialkylaminocarbonyl, diarylaminocarbonyl or arylalkylaminocarbonyl; carboxyl, or alkyl- or aryloxycarbonyl; aldehyde; aryl- or alkylcarbonyl; iminyl, or aryl- or alkyliminyl; sulfo; alkyl- or arylsulfonyl; hydroximinyl, or aryl- or alkoximinyl. In addition, two or more alkyl substituents may be combined to form fused heterocycle-alkyl ring systems. Substituents including heterocyclic groups (e.g., heteroaryloxy, and heteroaralkylthio) are defined by analogy to the above-described terms.

The term “heterocycloalkyl” as used herein refers to a heterocycle group that is joined to a parent structure by one or more alkyl groups as described above, e.g., 2-piperidylmethyl, and the like. The term “heterocycloalkyl” refers to a heteroaryl group that is joined to a parent structure by one or more alkyl groups as described above, e.g., 2-thienylmethyl, and the like.

For brevity, the term “aryl” when used in combination with other terms (e.g., aryloxy, arylthioxy, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like).

Each of the above terms (e.g., “alkyl,” “heteroalkyl,” “aryl” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) are generically referred to as “alkyl group substituents,” and they can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such radical. R′, R″, R′″ and R′″ each preferably independently refer to hydrogen, substituted or unsubstituted heteroalkyl, substituted or unsubstituted aryl, e.g., aryl substituted with 1-3 halogens, substituted or unsubstituted alkyl, alkoxy or thioalkoxy groups, or arylalkyl groups. When a compound of the invention includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are generically referred to as “aryl group substituents.” The substituents are selected from, for example: halogen, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″ and R″″ are preferably independently selected from hydrogen, substituted or unsubstituted alkyl, substituted or unsubstituted heteroalkyl, substituted or unsubstituted aryl and substituted or unsubstituted heteroaryl. When a compound of the invention includes more than one R group, for example, each of the R groups is independently selected as are each R′, R″, R′″ and R″″ groups when more than one of these groups is present. In the schemes that follow, the symbol X represents “R” as described above.

The aryl and heteroaryl substituents described herein are unsubstituted or optionally and independently substituted by H, halogen, cyano, sulfonic acid, carboxylic acid, nitro, alkyl, perfluoroalkyl, alkoxy, alkylthio, amino, monoalkylamino, dialkylamino or alkylamido.

Two of the substituents on adjacent atoms of the aryl or heteroaryl ring may optionally be replaced with a substituent of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)S—X—(CR″R′″)_{d}—, where s and d are independently integers of from 0 to 3, and X is —O—, —NR′—, —S—, —S(O)—, —S(O)₂—, or —S(O)₂NR′—. The substituents R, R′, R″ and R′″ are preferably independently selected from hydrogen or substituted or unsubstituted (C₁-C₆)alkyl.

As used herein, the term “heteroatom” includes oxygen (O), nitrogen (N), sulfur (S), phosphorus (P) and silicon (Si).

The term “amino” or “amine group” refers to the group —NR′R″ (or NRR′R″) where R, R′ and R″ are independently selected from the group consisting of hydrogen, alkyl, substituted alkyl, aryl, substituted aryl, aryl alkyl, substituted aryl alkyl, heteroaryl, and substituted heteroaryl. A substituted amine being an amine group wherein R′ or R″ is other than hydrogen. In a primary amino group, both R′ and R″ are hydrogen, whereas in a secondary amino group, either, but not both, R′ or R″ is hydrogen. In addition, the terms “amine” and “amino” can include protonated and quaternized versions of nitrogen, comprising the group —NRR′R″ and its biologically compatible anionic counterions.

The term “buffer” as used herein refers to a system that acts to minimize the change in acidity or basicity of the solution against addition or depletion of chemical substances.

The term “carbonyl” as used herein refers to the functional group —(C═O)—. However, it will be appreciated that this group may be replaced with other well-known groups that have similar electronic and/or steric character, such as thiocarbonyl (—(C═S)—); sulfinyl (—S(O)—); sulfonyl (—SO₂)—), phosphonyl (—PO₂—).

The term “acetic acid binding domain” as used herein refers to a domain that contains at least two terminal acetic acid groups, as defined below. The acetic acid binding domains contain nitrogen as the point of attachment for the acetic acid groups and the binding domain is attached to a linker at either a nitrogen or carbon atom depending on one of the three (I, II or III) formulas for the binding domain. Specifically, the acetic acid binding domains have formula (I) ⁻O₂CCH(R)N(CH₂CO₂⁻)₂, wherein R is a linker that is covalently bonded to the methine carbon atom (See, for example Compound 1), or formula (II) —N(CH₂CO₂⁻)₂ wherein the linker is covalently bonded to the nitrogen atom (See, for example Compound 12). Alternatively, the acetic acid binding domain has formula (III) (CH₂CO₂⁻)_{Z}N[(CH(R))ₛN(CH₂CO₂⁻)]_{T}(CH(R))_{S}N(CH₂CO₂⁻)_{Z} wherein the linker is attached to a methine carbon or nitrogen atom and Z is 1 or 2, S is 1 to 5 and T is 0 to 4. In all cases, the acetic acid binding domain contains at least two acetic acid groups and the nitrogen atom is the point of attachment for the acetic acid groups.

The term “acetic acid group” as used herein refers to the chemical formula (IV) —CH(R)CO₂⁻, which includes the protenated form —CH(R)CO₂H. R is independently H or a Linker, as defined below. When R is hydrogen the acetic acid group has the formula —CH₂CO₂⁻. When the linker of the fluorescent compound is attached to a methine carbon of an acetic acid group then R is the linker. When an acetic acid group is referred to, it is understood to be a terminal end of a compound, which allows for the negatively charged carboxy group of the acetic acid group to freely interact with a positively charged affinity-binding domain. When acetic acid groups are part of the binding domain, nitrogen is the point of attachment for the acetic acid groups. These binding domains are particularly useful for labeling and detecting poly-histidine affinity tags, e.g. ⁻O₂CCH(R)N(CH₂CO₂⁻)₂ wherein R is the point of attachment of the Linker.

The term “affinity” as used herein refers to the strength of the binding interaction of two molecules, such as a metal chelating compound and a metal ion or a positively charged moiety and a negatively charged moiety.

The term “affinity tag” as used herein refers to any known amino acid sequence fused to a protein of interest at either the amino terminal or carboxy terminal end of the protein (K. Terpe, Appl. Microbiol. Biotechnol (2003) 60:523-533). Typically, the affinity tag is used for isolation and or detection purposes. The “affinity tag” may optionally be in the middle of the protein of interest such that when the corresponding nucleic acid sequence is translated the affinity tag is fused in frame into the protein of interest. The amino acid residues form a peptide that has affinity for a chemical moiety, a metal ion or a protein. The affinity tag may have an overall positive, negative or neutral charge; typically the affinity tag has an overall positive or negative charge.

The term “affinity-tag-containing-fusion protein” as used herein refers to a fusion protein that contains a protein of interest and an affinity tag.

The term “aqueous solution” as used herein refers to a solution that is predominantly water and retains the solution characteristics of water. Where the aqueous solution contains solvents in addition to water, water is typically the predominant solvent.

The term “B binding domain”, “B” and “binding domain” as used herein refer to a component of the fluorescent compound that interacts directly or indirectly with the affinity tag of the fusion protein. The binding domain can be a chemical moiety that has an overall charge or a protein, provided the protein is not an antibody or a fragment thereof. The binding domain may be substituted to adjust the binding affinity, solubility or other physical properties of the fluorescent compound that the binding domain is covalently attached to. An important aspect of the invention is that the binding domain does not contain an arsenic atom.

The term “buffer” as used herein refers to a system that acts to minimize the change in acidity or basicity of the solution against addition or depletion of chemical substances.

The term “calmodulin” as used herein refers to a binding domain that when complexed with calcium binds the calmodulin affinity tag.

The term “calmodulin affinity tag” as used herein refers to the amino acid sequence that codes for calmodulin binding peptide and includes any corresponding peptides disclosed in U.S. Pat. Nos. 5,585,475; 6,117,976 and 6,316,409. The “calmodulin affinity tag” is fused to a protein of interest for the purposes of detection and purification.

The term “carrier molecule” as used herein refers to a fluorescent compound of the present invention that is covalently bonded to a biological or a non-biological component. Such components include, but are not limited to, an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof.

The term “complex” as used herein refers to the association of two or more molecules, usually by non-covalent bonding, e.g., the association between the negatively charged acetic acid groups and the positively charged histidine residues of a poly-histidine affinity tag.

The term “detectable response” as used herein refers to an occurrence of, or a change in, a signal that is directly or indirectly detectable either by observation or by instrumentation. Typically, the detectable response is an occurrence of a signal wherein the fluorophore is inherently fluorescent and does not produce a significant change in signal upon binding to a metal ion or biological compound. Alternatively, the detectable response is an optical response resulting in a change in the wavelength distribution patterns or intensity of absorbance, fluorescence or a change in light scatter, fluorescence lifetime, fluorescence polarization, or a combination of the above parameters.

The term “direct binding” as used herein refers to binding of the fluorescent compound to the affinity tag of the fusion protein with the proviso that a metal ion does not comprise the resulting complex. Typically the charged binding domain of the fluorescent compound has an affinity for the charged amino acid residues of the affinity tag wherein a stable non-covalent bond is formed between the compound and peptide.

The term “FLAG affinity tag” as used herein refers to the amino acid sequence DYKDDDDK and any corresponding peptide disclosed in U.S. Pat. Nos. 4,851,341 and 5,011,912, wherein the FLAG affinity tag is fused to a protein of interest.

The term “fluorescent compound” as used herein refers to a compound with the general formula A(B)n wherein A is a fluorophore, B is a binding domain comprising a chemical moiety, protein or fragment thereof that is capable of binding, directly or indirectly, to the affinity tag of the fusion protein wherein n is an integer from about 1 to about 6, with the proviso that the fluorescent compound does not comprise an antibody or fragment thereof. When the binding domain is a chemical moiety the fluorescent compound has the general formula A(L)m(B)n wherein L is a Linker that covalently attaches the fluorophore to the binding domain. The fluorescent compound of the present invention effectively non-covalently attaches a fluorophore to the fusion protein at the site of the affinity tag.

The term “fluorophore” as used herein refers to a compound that is inherently fluorescent or demonstrates a change in fluorescence upon binding to a biological compound or metal ion, i.e., fluorogenic. Numerous fluorophores are known to those skilled in the art and include, but are not limited to, coumarin, cyanine, acridine, anthracene, benzofuran, indole, borapolyazaindacene and xanthenes including fluorescein, rhodamine and rhodol as well as other fluorophores described in RICHARD P. HAUGLAND, MOLECULAR PROBES HANDBOOK OF FLUORESCENT PROBES AND RESEARCH CHEMICALS (9^{th} edition, CD-ROM, 2002).

The term “fusion protein” as used herein refers to a protein hybrid containing an affinity tag and a protein of interest or any amino acid sequence of interest. The affinity tag may be directly linked or indirectly linked to the fusion protein. When the affinity tag is indirectly linked there is preferably a cleavage site between the affinity tag and the protein of interest that facilitates recovery of the protein of interest free from the affinity tag. When a fusion protein containing a cleavage site comes into contact with an appropriate protease that is specific for the cleavage site, such as enterokinase, the fusion protein is cleaved into two polypeptides: the affinity tag and the protein of interest.

The term “Glu-Glu affinity tag” as used herein refers to the amino acid sequence EEEEYMPME or a fragment thereof that is fused to a protein of interest, either at an end or within the protein.

The term “glutathione” as used herein refers to a tripeptide, or derivative thereof, that specifically binds to the GST affinity tag and when part of a fluorescent compound of the present invention represents the binding domain of the fluorescent compound. Typically, “glutathione” refers to the tripeptide γ-glutamylcysteinylglycine, Glu-(Cys-Gly).

The term “GST affinity tag” as used herein refers to an amino acid sequence that encodes for all or part of glutathione S-transferase including any corresponding polypeptides disclosed in U.S. Pat. No. 5,654,176, that is fused to a protein of interest.

The term “poly-histidine affinity tag” as used herein refers to a non-natural consecutive sequence of histidine amino acid residues including any corresponding peptides disclosed in U.S. Pat. Nos. 5,284,933 and 5,310,663. Typically such sequences comprise four to ten histidine residues that are typically linked to the carboxy and/or amino terminal end of a protein of interest. Optionally, the poly-histidine affinity tag may be linked, in-frame, in the middle of the protein of interest.

The term “indirect binding” as used herein refers to the binding of the fluorescent compound to the affinity tag due to a third component, typically a polyvalent metal ion. The fluorescent compound and the affinity tag form a ternary complex with a metal ion wherein the metal ion binds both the affinity tag and the acetic acid groups of the fluorescent compound. The metal ion has affinity for both the binding domain and affinity tag and as such confers affinity to the binding domain for the affinity tag that would not be present without the metal ion. Alternatively, a metal ion has affinity for the binding domain that when bound induces a conformational change that confers affinity to the binding domain for the affinity tag. Thus, in this instance, the metal ion may not have affinity for the affinity tag; however, the metal ion will induce the binding domain to have affinity for the affinity tag.

The term “isolated,” as used herein refers to, a preparation of peptide, protein or protein complex that is essentially free from contaminating proteins that normally would be present in association with the peptide, protein or complex, e.g., in a cellular mixture or milieu in which the protein or complex is found endogenously. In addition “isolated” also refers to the further separation from reagents used to isolate the peptide, protein or complex from cellular mixture. Thus, an isolated fusion protein may be isolated from cellular components and optionally from the fluorescent compounds of the present invention that normally would contaminate or interfere with the study of the complex in isolation, for example while screening for modulators thereof.

The term “kit” as used refers to a packaged set of related components, typically one or more compounds or compositions.

The term “linker” or “L”, as used herein, refers to a single covalent bond or a series of stable covalent bonds incorporating 1-30 nonhydrogen atoms selected from the group consisting of C, N, O, S and P that covalently attach the fluorophore to the binding domain of the fluorescent compounds. In addition, the linker covalently attaches a carrier molecule or solid support to the present fluorescent compounds. Exemplary linking members include a moiety that includes —C(O)NH—, —C(O)O—, —NH—, —S—, —O—, and the like. A “cleavable linker” is a linker that has one or more cleavable groups that may be broken by the result of a reaction or condition. The term “cleavable group” refers to a moiety that allows for release of a portion, e.g., a reporter molecule, carrier molecule or solid support, of a conjugate from the remainder of the conjugate by cleaving a bond linking the released moiety to the remainder of the conjugate. Such cleavage is either chemical in nature, or enzymatically mediated. Exemplary enzymatically cleavable groups include natural amino acids or peptide sequences that end with a natural amino acid.

In addition to enzymatically cleavable groups, it is within the scope of the present invention to include one or more sites that are cleaved by the action of an agent other than an enzyme. Exemplary non-enzymatic cleavage agents include, but are not limited to, acids, bases, light (e.g., nitrobenzyl derivatives, phenacyl groups, benzoin esters), and heat. Many cleaveable groups are known in the art. See, for example, Jung et al., Biochem. Biophys. Acta, 761: 152-162 (1983); Joshi et al., J. Biol. Chem., 265:14518-14525 (1990); Zarling et al., J. Immunol., 124: 913-920 (1980); Bouizar et al., Eur. J. Biochem., 155: 141-147 (1986); Park et al., J. Biol. Chem., 261: 205-210 (1986); Browning et al., J. Immunol., 143: 1859-1867 (1989). Moreover a broad range of cleavable, bifunctional (both homo- and hetero-bifunctional) spacer arms are commercially available.

An exemplary cleavable group, an ester, is cleavable group that may be cleaved by a reagent, e.g. sodium hydroxide, resulting in a carboxylate-containing fragment and a hydroxyl-containing product.

The term “metal chelator” or “metal chelating moiety” as used herein refers to a chemical compound that combines with a metal ion to form a chelate structure.

The term “metal ion” as used herein refers to any metal ion that has an affinity for an affinity tag and/or a binding domain and that can be used to indirectly complex the fluorescent compound and the fusion protein together. Such metal ions include, but are not limited to, Ni²⁺, Co²⁺, Zn²⁺, Cu²⁺, Al³⁺, Ca²⁺, Ac³⁺, Fe³⁺ and Ga³⁺.

The term “NTA” as used herein refers to the metal chelating group Nα, Noα-bis(carboxymethyl)-lysine and derivatives thereof. Such derivatives include nitriloacetic acid.

The term “poly-arginine affinity tag” as used herein refers to a consecutive sequence, typically 4-6, of arginine residues (Nock et al (1997) FEBS Lett. 414(2):233-238).

The terms “protein” and “polypeptide” are used herein in a generic sense to include polymers of amino acid residues of any length. The term “peptide” is used herein to refer to polypeptides having less than 250 amino acid residues, typically less than 100 amino acid residues. The terms apply to amino acid polymers in which one or more amino acid residues are an artificial chemical analogue of a corresponding naturally occurring amino acid, as well as to naturally occurring amino acid polymers.

The term “protein of interest” as used herein refers to any protein to which an affinity tag is fused to for the purpose of detection, isolation, labeling, tagging, monitoring and/or purification.

The term “reactive group” as used herein refers to a group that is capable of reacting with another chemical group to form a covalent bond, i.e. is covalently reactive under suitable reaction conditions, and generally represents a point of attachment for another substance. The reactive group is a moiety, such as carboxylic acid or succinimidyl ester, on the compounds of the present invention that is capable of chemically reacting with a functional group on a different compound to form a covalent linkage. Reactive groups generally include nucleophiles, electrophiles and photoactivatable groups.

Exemplary reactive groups include, but are not limited to, olefins, acetylenes, alcohols, phenols, ethers, oxides, halides, aldehydes, ketones, carboxylic acids, esters, amides, cyanates, isocyanates, thiocyanates, isothiocyanates, amines, hydrazines, hydrazones, hydrazides, diazo, diazonium, nitro, nitriles, mercaptans, sulfides, disulfides, sulfoxides, sulfones, sulfonic acids, sulfinic acids, acetals, ketals, anhydrides, sulfates, sulfenic acids isonitriles, amidines, imides, imidates, nitrones, hydroxylamines, oximes, hydroxamic acids thiohydroxamic acids, allenes, ortho esters, sulfites, enamines, ynamines, ureas, pseudoureas, semicarbazides, carbodiimides, carbamates, imines, azides, azo compounds, azoxy compounds, and nitroso compounds. Reactive functional groups also include those used to prepare bioconjugates, e.g., N-hydroxysuccinimide esters, maleimides and the like. Methods to prepare each of these functional groups are well known in the art and their application to or modification for a particular purpose is within the ability of one of skill in the art (see, for example, Sandler and Karo, eds. ORGANIC FUNCTIONAL GROUP PREPARATIONS, Academic Press, San Diego, 1989).

The term “salt thereof,” as used herein includes salts of the agents of the invention and their conjugates, which are preferably prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of base addition salts include sodium, potassium, calcium, ammonium, organic amino, or magnesium, or a similar salt. When compounds of the present invention contain relatively basic functionalities, acid addition salts can be obtained by contacting the neutral form of such compounds with a sufficient amount of the desired acid, either neat or in a suitable inert solvent. Examples of addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The term “sample” as used herein refers to any material that may contain fusion proteins, as defined above. Typically, the sample comprises endogenous host cell proteins. The sample may be in an aqueous solution, a viable cell culture or immobilized on a solid or semi solid surface such as a polymeric gel, polymeric bead, membrane blot or on a microarray.

The term “solid support,” as used herein, refers to a material that is substantially insoluble in a selected solvent system, or which can be readily separated (e.g., by precipitation) from a selected solvent system in which it is soluble. Solid supports useful in practicing the present invention can include groups that are activated or capable of activation to allow selected species to be bound to the solid support. Solid supports may be present in a variety of forms, including a chip, wafer or well, onto which an individual, or more than one compound, of the invention is bound such as a polymeric bead or particle.

### The Compounds

In general, for ease of understanding the present invention, the fluorescent compounds and corresponding substituents will first be described in detail, followed by the many and varied methods in which the compounds find uses, which is followed by exemplified methods of use and synthesis of novel compounds that are particularly advantageous for use with the methods of the present invention.

The present invention provides fluorescent compounds that have an affinity for a number of affinity tags. When the binding domain is a protein, typically there is a short linker, less than 10 nonhydrogen atoms that covalently attach the fluorophore to the protein-binding domain. The protein-binding domain may interact directly or indirectly through a metal ion with the affinity tag. When the binding domain is a charged chemical moiety the fluorescent compounds of the present invention have the general formula A(L)m(B)n wherein A is a fluorophore, L is a Linker, B is a binding domain, m is an integer from 1 to 4 and n is an integer from 1 to 6. By selection of an appropriate binding domain, a corresponding affinity tag can be selectively and non-covalently labeled with a fluorophore. The fluorophore typically has a passive role in the affinity of the binding domain for the affinity tag, although the fluorophore may be substituted to alter the affinity of the covalently attached binding domain. However, we have found that fluorophores that are substituted by sulfonated groups tend to reduce the selectivity of the fluorescent compound for the affinity tag. Therefore, one skilled in the art will appreciate that any fluorophore, or derivative thereof, can be covalently linked using an appropriate Linker(s) to a specific binding domain resulting in a significant advancement in the ability to fluorescently detect fusion proteins that contain an affinity tag.

### 1. Fluorophores

A fluorophore of the present invention is any chemical moiety that exhibits an absorption maximum beyond 280 nm, and when covalently linked to a binding domain of the present invention forms a fluorescent compound. The covalent linkage can be a single covalent bond or a combination of stable chemical bonds. The covalent linkage attaching the fluorophore to the binding domain is typically a substituted alkyl chain that incorporates 1-30 nonhydrogen atoms selected from the group consisting of C, N, O, S and P. Optionally, the linker can be a single covalent bond or the alkyl chain can incorporate a benzene ring, aryl, substituted aryl, heteroaryl or substituted heteroaryl ring.

Fluorophores of the present invention include, without limitation; a pyrene, an anthracene, a naphthalene, an acridine, a stilbene, an indole or benzindole, an oxazole or benzoxazole, a thiazole or benzothiazole, a 4-amino-7-nitrobenz-2-oxa-1,3-diazole (NBD), a carbocyanine (including any corresponding compounds in U.S. Ser. Nos. 09/557,275; 09/968,401 and 09/969,853 and U.S. Pat. Nos. 6,403,807; 6,348,599; 5,486,616; 5,268,486; 5,569,587; 5,569,766; 5,627,027 and 6,048,982), a carbostyryl, a porphyrin, a salicylate, an anthranilate, an azulene, a perylene, a pyridine, a quinoline, a borapolyazaindacene (including any corresponding compounds disclosed in U.S. Pat. Nos. 4,774,339; 5,187,288; 5,248,782; 5,274,113; and 5,433,896), a xanthene (including any corresponding compounds disclosed in U.S. Pat. Nos. 6,162,931; 6,130,101; 6,229,055; 6,339,392; 5,451,343 and U.S. Ser. No. 09/922,333), an oxazine or a benzoxazine, a carbazine (including any corresponding compounds disclosed in U.S. Pat. No. 4,810,636), a phenalenone, a coumarin (including an corresponding compounds disclosed in U.S. Pat. Nos. 5,696,157; 5,459,276; 5,501,980 and 5,830,912), a benzofuran (including an corresponding compounds disclosed in U.S. Pat. Nos. 4,603,209 and 4,849,362) and benzphenalenone (including any corresponding compounds disclosed in U.S. Pat. No. 4,812,409) and derivatives thereof. As used herein, oxazines include resorufins (including any corresponding compounds disclosed in U.S. Pat. No. 5,242,805), aminooxazinones, diaminooxazines, and their benzo-substituted analogs.

Where the dye is a xanthene, the dye is optionally a fluorescein, a rhodol (including any corresponding compounds disclosed in U.S. Pat. Nos. 5,227,487 and 5,442,045), a rosamine or a rhodamine (including any corresponding compounds in U.S. Pat. Nos. 5,798,276; 5,846,737; 5,847,162; 6,017,712; 6,025,505; 6,080,852; 6,716,979; 6,562,632). As used herein, fluorescein includes benzo- or dibenzofluoresceins, seminaphthofluoresceins, or naphthofluoresceins. Similarly, as used herein rhodol includes seminaphthorhodafluors (including any corresponding compounds disclosed in U.S. Pat. No. 4,945,171).

Preferred fluorophores of the invention include xanthene, coumarin, cyanine, acridine, anthracene, benzofuran, indole and borapolyazaindacene. Most preferred are cyanine, rhodamine, borapolyazaindacene, coumarin and benzofuran. The choice of the fluorophore attached to the binding domain will determine the fluorescent compound's absorption and fluorescence emission properties. It is an aspect of the present invention that the fluorophore not be sulfonated.

Typically the fluorphore contains one or more aromatic or heteroaromatic rings, that are optionally substituted one or more times by a variety of substituents, including without limitation, halogen, nitro, sulfo, cyano, alkyl, perfluoroalkyl, alkoxy, alkenyl, alkynyl, cycloalkyl, arylalkyl, acyl, aryl or heteroaryl ring system, benzo, or other substituents typically present on chromophores or fluorophores known in the art.

In an exemplary embodiment, the fluorophores are independently substituted by substituents selected from the group consisting of hydrogen, halogen, amino, substituted amino, alkyl, substituted alkyl, aryl, substituted aryl, heteroaryl, substituted heteroaryl, alkoxy, sulfo, reactive group and carrier molecule. In another embodiment, the xanthene fluorophores of this invention comprise both compounds substituted and unsubstituted on the carbon atom of the central ring of the xanthene by substituents typically found in the xanthene-based dyes such as phenyl and substituted-phenyl moieties. Most preferred fluorophores are rhodamine, fluorescein, rhodal, rosamine and derivatives thereof.

### 2. Linkers

As described above, the fluorophores of the present invention are covalently attached to a binding domain by a linker to form the fluorescent compounds of the present invention. The Linker typically incorporates 1-30 nonhydrogen atoms selected from the group consisting of C, N, O, S and P. The linker is typically a substituted alkyl or a substituted cycloalkyl. Alternatively, the fluorophore may be directly attached (where linker is a single bond) to the binding domain or the alkyl may contain a benzene ring. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. Typically the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. Typically the linker is a combination of single carbon-carbon bonds and carboxamide, sulfonamide or thioether bonds. The bonds of the linker typically result in the following moieties that can be found in the linker: ether, thioether, carboxamide, thiourea, sulfonamide, urea, urethane, hydrazine, alkyl, aryl, heteroaryl, alkoxy, cycloalkyl and amine moieties. Examples of typical fluorescent compounds incorporate the following three (V, VI and VII) Linker formulas: Formula (V) —(CH₂)ₑC(X)NH(CH₂)ₑ(NHC(X)(CH₂)ₑ)_{d}—, Formula (VI) —((C₆R″₄)O)_{d}(CH₂)ₑ(C(X)NH(CH₂)ₑ)(NH)_{d}C(X)NH(C₆R′₄)(CH₂)ₑ— and Formula (VII) —(O)_{d}(CH₂)_{f}O(C₆R″₄)— wherein X is O or S, d is 0-1, e is 1-6, f is 2 or 3, and R″ is independently H, halogen, alkoxy or alkyl. It is understood that X, d, e and are independently selected within a linker.

Alternatively, the linker comprises a heteroaryl moiety wherein one of the carbon atoms in an aryl group is replaced by a heteroatom such as nitrogen. One such example includes the Formula (XYZ) —(CH₂)ₑNC₅H₉C(X)(NH)_{d}(CH₂)ₑ—. In one embodiment, the first e is 0 and the nitrogen atom also forms part of the fluorophore, such as when the fluorophore is a rhodamine.

Furthermore, a selected embodiment of the present invention is the following fluorescent compound formulas (VIII, IX and X): Formula (VIII) (A)-[(CH₂)ₑC(X)NH(CH₂)ₑ(NHC(X)(CH₂)ₑ)_{d}]ₘ—(B)ₙ;

Formula (IX) (A)-((C₆R″₄)O)_{d}(CH₂)ₑ(C(X)NH(CH₂)ₑ)(NH)_{d}C(X)NH(C₆R″₄)(CH₂)ₑ—(B) and Formula (X) (A)-(O)_{d}(CH₂)_{f}O(C₆R″₄)—(B) wherein m is an integer from 1 to 4, m is an integer from 1 to 6, A is a fluorophore and B is a binding domain. Particularly preferred is Formula (VIII) wherein d is 0, e is 1 to 4, X is O, m Is 2 and n is 2 or Formula (VIII) wherein d is 1 and e is 1 or 2. Preferred embodiments of Formula (X) is when d is 0, f is 2, or a variation of Formula (X) having the Formula (XI) (B)(L)(A)-(O)_{d}(CH₂)_{f}O(C₆R″₄)—(B) wherein L is a single covalent bond, B is a binding domain, A is fluorophore, d is 1 and f is 2.

In another aspect, a preferred fluorescent compound has the formula (XYZ2) (A)-[NC₅H₉C(O)NH(CH₂)₄]₂—(B)₂.

Any combination of linkers may be used to attach the fluorophore and the binding domain together, typically a fluorophore will have one or two linkers attached that may be the same or different. In addition, a linker may have more than one binding domain per linker. The linker may also be substituted to alter the physical properties of the fluorescent compound, such as binding affinity of the binding domain and spectral properties of the fluorophore. For fluorescent compounds that have an affinity for the poly-histidine affinity tag, the linker typically incorporates an oxygen atom due to its ability to increase the affinity of the acetic acid binding domain, described below, for the affinity tag. This feature of the linker is especially true for fluorescent compound Formula (XI) (B)(L)(A)-(O)_{d}(CH₂)_{f}O(C₆R″₄)—(B). Thus, an important feature of the linker is to alter the binding affinity of the binding domain by increasing the affinity with the incorporation of oxygen into the linker.

The linker can also have other substituents that alter the binding affinity of the binding domain. The benzene ring (C₆R″₄) of Formula (XI) is typically substituted with a halogen, preferably chlorine or fluorine, which tunes the affinity of the binding domain. These halogen substituents appear to lower the affinity of binding domain but increase the specificity of the binding domain for the affinity tag resulting in overall increased sensitivity of the fluorescent compound for the affinity tag. Thus, linker substituents function to tune the binding affinity of the fluorescent compound to optimize the sensitivity of the binding domain for the affinity tag

Another important feature of the linker is to provide an adequate space between the fluorophore and the binding domain so as to prevent the fluorophore from providing a steric hindrance to the binding of the affinity tag for the binding domain of the fluorescent compound. Thus, when a binding domain is attached to the fluorophore by a single covalent bond there is typically another linker containing an oxygen atom attached to the same fluorophore at a different position to increase the affinity of both binding domains for the affinity tag. Therefore, the linker of the present fluorescent compounds is important for (1) coupling the fluorophore to the binding domain, (2) providing an adequate space between the fluorophore and the binding domain so as not to sterically hinder the affinity of the binding domain and the affinity tag and (3) altering the affinity of the binding domain for the affinity tag either by the choice of the atoms of the linker or indirectly by addition of substituents to the linker.

The covalent bond of the linker to A or B should typically not be unintentionally cleaved by chemical or enzymatic reactions during the assay. In some cases it may be desirable to cleave the linker from the fluorophore moiety or the binding domain, or from the reactive group, for example to facilitate release from an affinity column, wherein the fluorescent compound is covalent attached to a carrier molecule or solid support, or for sequencing purposes. Thus, the linker can be cleavable, for example, by chemical, thermal or photochemical reaction. Photocleavable groups in the linker may include the 1-(2-nitrophenyl)-ethyl group. Thermally labile linkers may, for example, be a double-stranded duplex formed from two complementary strands of nucleic acid, a strand of a nucleic acid with a complementary strand of a peptide nucleic acid, or two complementary peptide nucleic acid strands which will dissociate upon heating. Cleavable linkers also include those having disulfide bonds, acid or base labile groups, including among others, diarylmethyl or trimethylarylmethyl groups, silyl ethers, carbamates, oxyesters, thiesters, thionoesters, and α-fluorinated amides and esters. Enzymatically cleavable linkers can contain, for example, protease-sensitive amides or esters, β-lactamase-sensitive β-lactam analogs and linkers that are nuclease-cleavable, or glycosidase-cleavable.

### 3. Binding Domains

The binding domain of the present fluorescent compounds, include without limitation, charged chemical moieties, a protein or fragments thereof that are capable of non-covalently binding to an affinity tag of the present invention. The binding domain, either independently or when complexed with a metal ion, has specific and selective affinity for an affinity tag containing fusion protein. The fluorescent compounds, A(L)m(B)n, may have more than one linker and more than one binding domain, which may or may not be the same. We have found that bis-chelates are particularly useful for the detection of histidine tagged fusion proteins in gels. Preferably, the binding domains are all selective for the same affinity tag, however for certain applications it may be desirable to have one fluorophore linked to binding domains that have selective affinity for different affinity tags. In this manner, selection and orientation of the binding domain relative to the fluorophore is critical for the specificity, sensitivity and selectivity of the binding domain.

The present invention contemplates protein and peptide-binding domains that are not antibodies or fragments thereof. Thus, an aspect of the present invention is affinity tags that are selective for such proteins, and these include without limitation, GST, calmodulin, maltose-binding, and chitin-binding affinity tags. These peptides bind the glutathione tripeptide, calmodulin protein, maltose and chitin respectively. When these polypeptides are attached by a linker to a fluorophore, they function to site-specifically label these affinity tag containing fusion proteins.

Calmodulin selectively and with high affinity binds calcium ions, the calcium ions then induce a conformation change that causes the protein to have affinity for the calmodulin affinity tag (Hentz N G et al. (1996) Anal Chem 68:1550-5; Zheng C F et al. (1997) 186:55-60). A fluorophore of the present invention that is covalently attached to calmodulin effectively attaches the fluorophore to the calmodulin affinity tag and subsequently a protein of interest. Thus, a staining solution specific for calmodulin affinity tag containing fusion proteins would include, at a minimum, a fluorescent compound comprising calmodulin and calcium ions.

In contrast, the glutathione tripeptide binds directly to the GST affinity tag (Kaplan W et al (1997) Protein Sci. 6:399-406; Lew A M et al (1991) J. Immunol. Methods 136:211-9). A fluorescent compound covalently attached to glutathione effectively attaches a fluorophore to a GST affinity tag containing fusion protein. In this way, fluorescent compounds comprising glutathione, provide an effective means for detecting such fusion proteins in a gel or solution, a means not previously feasible with currently known compounds, See Example 20. Thus, an aspect of the invention is detection of GST affinity tag containing fusion proteins with a fluorescent compound that comprises the tripeptide glutathione. Preferred fluorescent compounds comprise a xanthene fluorophore.

An important aspect of the present invention includes charged chemical moieties that have affinity for an affinity peptide. These moieties include, without limitation, acetic acid groups, phosphates and sulfates. Particularly preferred are binding domains that have affinity for positively charged affinity tags such as poly-histidine or poly-arginine affinity tag containing fusion proteins. These binding domains typically contain terminal acetic acid groups. The acetic acid binding domains contain nitrogen as the point of attachment for the acetic acid groups and the binding domain is attached to a linker at either a nitrogen or carbon atom depending on one of the three (I, II or III) formulas for the binding domain. Specifically, the acetic acid binding domains have formula (I)⁻O₂CCH(R)N(CH₂CO₂⁻)₂, wherein R is a linker that is covalently bonded to the methine carbon atom (See, for example Compound 1), or formula (II) —N(CH₂CO₂)₂ wherein the linker is covalently bonded to a nitrogen atom (See, for example Compound 12). Alternatively, the acetic acid binding domain has formula (III) (CH₂CO₂⁻)_{Z}N[(CH(R))_{S}N(CH₂CO₂⁻)]_{T}(CH(R))_{S}N(CH₂CO₂⁻)_{Z} wherein the linker is attached to a methine carbon or nitrogen atom and Z is 1 or 2, S is 1 to 5 and T is 0 to 4. In all cases, the acetic acid binding domain contains at least two acetic acid groups and a nitrogen atom is the point of attachment for the acetic acid groups. When a binding domain that contains only two acetic acid groups is attached to a fluorophore either (1) another acetic acid binding domain is also attached to the fluorophore or (2) an acetic acid group is attached by a linker to the fluorophore. This is because the fluorescent compounds with at least three acetic acid groups is preferable for providing selective and sensitive affinity for the poly-histidine affinity tag.

The acetic acid binding domains are typically part of a metal chelating moiety such as BAPTA, IDA, NTA, DTPA and TTHA. BAPTA, as used herein, refers to analogs, including derivatives, of the metal chelating moiety (1,2-bis(2-aminophenoxy)ethane-N,N,N′,N′-tetraacetic acid) and salts thereof including any corresponding compounds disclosed in U.S. Pat. Nos. 4,603,209; 4,849,362; 5,049,673; 5,453,517; 5,459,276; 5,516,911; 5,501,980; and 5,773,227. IDA, as used herein, refers to imidodiacetic acid compounds and derivatives thereof. DTPA, as used herein, refers to diethylenetriamine pentaacetic acid compounds and derivatives thereof including any corresponding compounds disclosed in U.S. Pat. Nos. 4,978,763 and 4,647,447. NTA, as used herein, refers to Nα, Nα-bis(carboxymethyl)-lysine and derivatives thereof, such derivatives including nitriloacetic acid. TTHA, as used herein, refers to triethylenetetramine hexaacetic acid and derivatives thereof.

The acetic acid binding domain may comprise the entire metal chelating moiety or only be part of such a moiety. A binding domain that encompasses an entire chelating moiety is represented by the formulas (I) ⁻O₂CCH(R)N(CH₂CO₂⁻)₂, and (III) (CH₂CO₂⁻)_{Z}N[(CH(R))_{S}N(CH₂CO₂⁻)]_{T}(CH(R))_{S}N(CH₂CO₂⁻)_{Z} wherein these formulas comprise the chelating moieties NTA (Formula I), DTPA and TTHA (Formula II). The binding domain having the formula (II) N(CH₂CO₂)₂ comprises, in part, the chelating moieties IDA and BAPTA. When a binding domain is only part of a chelating moiety such as BAPTA, the remaining part of the chelating moiety comprises the linker of a fluorescent compound or the fluorophore. This is demonstrated by the fluorescent compound Formula (X) (A)-(O)_{d}(CH₂)_{f}O(C₆R″₄)—(B), wherein the represented linker is part of the BAPTA chelating moiety. The remaining phenyl ring of the BAPTA moiety, when present, is typically part of the fluorophore, as demonstrated by Compound 12.

Due to the inclusion of chelating moieties in the binding domain and/or linker of the fluorescent compounds these moieties can be optionally substituted to adjust the binding affinity, solubility, or other physical properties of the compound. This is particularly true for the BAPTA chelating moiety wherein the benzene ring (C₆R″₄) of the linker Formula (VII) —(O)_{d}(CH₂)_{f}O(C₆R″₄)— is optionally substituted. Particularly advantageous substitutions are halogen substituents, especially fluorine and chlorine. Without wishing to be bound by a theory, it appears that these substituents, as electron withdrawing groups, tune the affinity of the binding domain for the affinity tag or a metal ion resulting in increased stability of the complex.

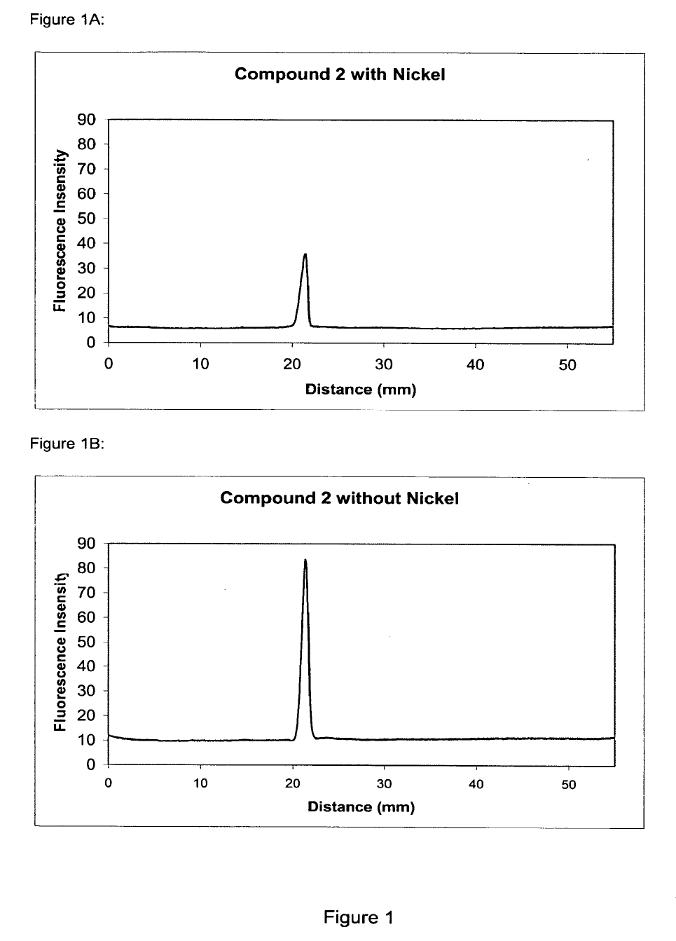

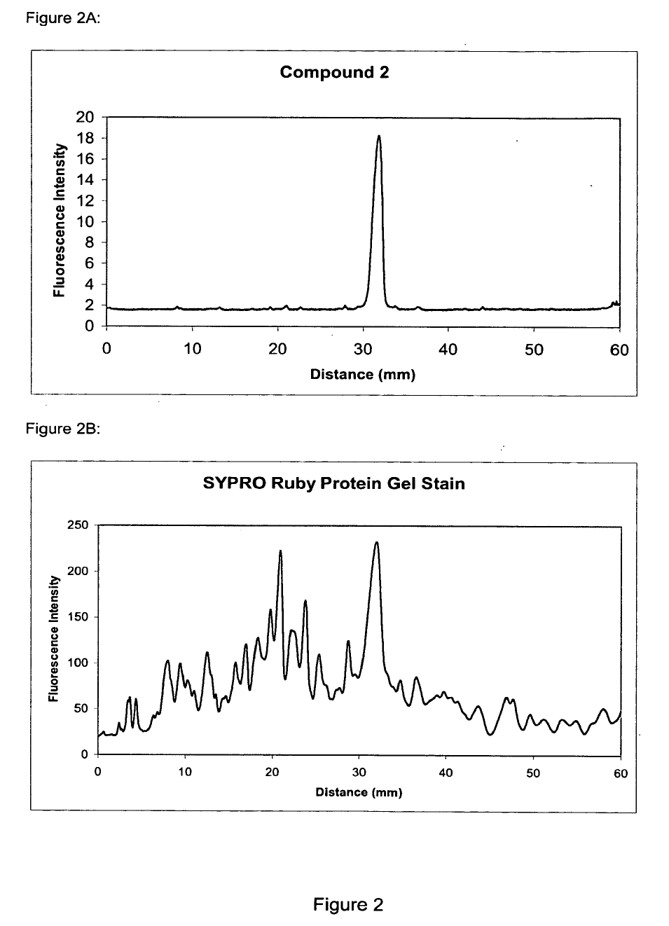

In addition, because the acetic acid binding domain contains all or part of a number of chelating moieties these binding domains also have affinity for metal ions. This aspect of the binding domain is useful for certain fluorescent compounds. However we have unexpectedly discovered that nickel ions are not necessary for the detection of poly-histidine affinity tag containing fusion proteins (FIG. 1). While this is an important aspect of the present invention, for some compounds, inclusion of the metal ion in a staining solution may be desirable. This is because for certain compounds, inclusion of metal ions into a staining solution or pre-complexed with the fluorescent compound may stabilize the complex of the fluorescent compound and the affinity tag containing fusion protein, See Example XYZ. Thus, for certain compounds, the inclusion of a metal ion is beneficial, See example XYZ2. In this instance the staining solution is preferably neutral or slightly basic. Alternatively, as demonstrated in FIGS. 1 and 2, the acetic acid binding domain has selective affinity for the poly-histidine affinity tag due to the negative charge of the acetic acid groups and the positive charge of the poly-histidine affinity tag at a neutral or mildly acidic pH, and in fact, as FIG. 1 demonstrates an increase in signal intensity is obtained when the staining solution does not contain nickel ions.

### 4. Reactive Groups, Carrier Molecules and Solid Supports

The present fluorescent compounds, in certain embodiments, are chemically reactive wherein the compounds comprise a reactive group. In this instance the reactive group is used to facilitate covalent attachment of the fluorophore to the binding domain, see Example XYZ. In a further embodiment, the compounds comprise a carrier molecule or solid support, which can be useful for purification or additional detection purposes. These substituents, reactive groups, carrier molecules, and solid supports, comprise a linker, as described above, that is used to covalently attach the substituents to any of the moieties of the present compounds having the formula (A)(B)n or A(L)m(B)n. The solid support, carrier molecule or reactive group may be directly attached (where linker is a single bond) to the moieties or attached through a series of stable bonds, as disclosed above.

In another exemplary embodiment of the invention, the present compounds are chemically reactive, and are substituted by at least one reactive group. The reactive group functions as the site of attachment for another moiety, such as a binding domain, carrier molecule or a solid support, wherein the reactive group chemically reacts with an appropriate reactive or functional group on the binding domain, carrier molecule or solid support. Thus, in another aspect of the present invention the fluorescent compounds comprise the binding domain, linker, fluorophore, a reactive group moiety and optionally a carrier molecule and/or a solid support.

In an exemplary embodiment, the compounds of the invention further comprise a reactive group which is a member selected from an acrylamide, an activated ester of a carboxylic acid, a carboxylic ester, an acyl azide, an acyl nitrile, an aldehyde, an alkyl halide, an anhydride, an aniline, an amine, an aryl halide, an azide, an aziridine, a boronate, a diazoalkane, a haloacetamide, a haloalkyl, a halotriazine, a hydrazine, an imido ester, an isocyanate, an isothiocyanate, a maleimide, a phosphoramidite, a photoactivatable group, a reactive platinum complex, a silyl halide, a sulfonyl halide, and a thiol. In a particular embodiment the reactive group is selected from the group consisting of carboxylic acid, succinimidyl ester of a carboxylic acid, hydrazide, amine and a maleimide. In exemplary embodiment, at least one member selected from A, L or B comprises a reactive group. Preferably, at least one of A or L comprises a reactive group. In another aspect, B comprises a reactive group. Alternatively, if the present compound comprises a carrier molecule or solid support a reactive group may be covalently attached independently to those substituents, allowing for further conjugation to a fluorophore, binding domain, carrier molecule or solid support.

In one aspect, the compound comprises at least one reactive group that selectively reacts with an amine group. This amine-reactive group is selected from the group consisting of succinimidyl ester, sulfonyl halide, tetrafluorophenyl ester and iosothiocyanates. This is particularly useful for covalently attaching the acetic acid binding domain to the present fluorescent compounds. Thus, in one aspect, the present compounds form a covalent bond with an amine-containing binding domain or alternatively with an amine-containing molecule in a sample. In another aspect, the compound comprises at least one reactive group that selectively reacts with a thiol group. This thiol-reactive group is selected from the group consisting of maleimide, haloalkyl and haloacetamide (including any reactive groups disclosed in U.S. Pat. Nos. 5,362,628; 5,352,803 and 5,573,904).

The pro-reactive groups are synthesized during the formation of the fluorophore, linker and carrier molecule and solid support containing compounds to provide chemically reactive fluorescent compounds. In this way, compounds incorporating a reactive group can be covalently attached to a wide variety of binding domains, carrier molecules or solid supports that contain or are modified to contain functional groups with suitable reactivity, resulting in chemical attachment of the components. In an exemplary embodiment, the reactive group of the compounds of the invention and the functional group of the binding domain, carrier molecule or solid support comprise electrophiles and nucleophiles that can generate a covalent linkage between them. Alternatively, the reactive group comprises a photoactivatable group, which becomes chemically reactive only after illumination with light of an appropriate wavelength. Typically, the conjugation reaction between the reactive group and the binding domain, carrier molecule or solid support results in one or more atoms of the reactive group being incorporated into a new linkage attaching the present compound of the invention to the binding domain, carrier molecule or solid support. Selected examples of functional groups and linkages are shown in Table 1, where the reaction of an electrophilic group and a nucleophilic group yields a covalent linkage.

TABLE 1
Examples of some routes to useful covalent linkages

| Electrophilic Group | Nucleophilic Group | Resulting Covalent Linkage |
|---|---|---|
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COΩ, where Ω is a good leaving group (e.g., succinimidyloxy (—OC₄H₄O₂) sulfosuccinimidyloxy (—OC₄H₃O₂—SO₃H), -1-oxybenzotriazolyl(—OC₆H₄N₃); or an aryloxy group or aryloxy substituted one or more times by electron withdrawing substituents such as nitro, fluor, chloro, cyano, or trifluoromethyl, orcombinations thereof, used to form activated aryl esters; or a carboxylic acid activated by a carbodiimide to form an anhydride or mixed anhydride —OCOR^{a} or —OCNR^{a}NHR^{b}, where R^{a} and R^{b}, which may be the same or different, are C₁-C₆ alkyl, C₁-C₆ perfluoroalkyl, or C₁-C₆ alkoxy; or cyclohexyl, 3-dimethylaminopropyl, or N-morpholinoethyl).
**Acyl azides can also rearrange to isocyanates

Choice of the reactive group used to attach the compound of the invention to the substance to be conjugated typically depends on the reactive or functional group on the substance to be conjugated and the type or length of covalent linkage desired. The types of functional groups typically present on the binding domains, carrier molecule or solid support include, but are not limited to, amines, amides, thiols, alcohols, phenols, aldehydes, ketones, phosphates, imidazoles, hydrazines, hydroxylamines, disubstituted amines, halides, epoxides, silyl halides, carboxylate esters, sulfonate esters, purines, pyrimidines, carboxylic acids, olefinic bonds, or a combination of these groups. A single type of reactive site may be available on the substance (typical for polysaccharides or silica), or a variety of sites may occur (e.g., amines, thiols, alcohols, phenols), as is typical for proteins.

Typically, the reactive group will react with an amine, a thiol, an alcohol, an aldehyde, a ketone, or with silica. Preferably, reactive groups react with an amine or a thiol functional group, or with silica. In one embodiment, the reactive group is an acrylamide, an activated ester of a carboxylic acid, an acyl azide, an acyl nitrile, an aldehyde, an alkyl halide, a silyl halide, an anhydride, an aniline, an aryl halide, an azide, an aziridine, a boronate, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as described in U.S. Pat. No. 5,714,327.

Where the reactive group is an activated ester of a carboxylic acid, such as a succinimidyl ester of a carboxylic acid, a sulfonyl halide, a tetrafluorophenyl ester or an isothiocyanates, the resulting compound is particularly useful for preparing conjugates of carrier molecules such as proteins, nucleotides, oligonucleotides, or haptens. Where the reactive group is a maleimide, haloalkyl or haloacetamide (including any reactive groups disclosed in U.S. Pat. Nos. 5,362,628; 5,352,803 and 5,573,904 (supra)) the resulting compound is particularly useful for conjugation to thiol-containing substances. Where the reactive group is a hydrazide, the resulting compound is particularly useful for conjugation to periodate-oxidized carbohydrates and glycoproteins, and in addition is an aldehyde-fixable polar tracer for cell microinjection. Where the reactive group is a silyl halide, the resulting compound is particularly useful for conjugation to silica surfaces, particularly where the silica surface is incorporated into a fiber optic probe subsequently used for remote ion detection or quantitation.

In a particular aspect, the reactive group is a photoactivatable group such that the group is only converted to a reactive species after illumination with an appropriate wavelength. An appropriate wavelength is generally a UV wavelength that is less than 400 nm. This method provides for specific attachment to only the target molecules, either in solution or immobilized on a solid or semi-solid matrix. Photoactivatable reactive groups include, without limitation, benzophenones, aryl azides and diazirines.

Preferably, the reactive group is a photoactivatable group, succinimidyl ester of a carboxylic acid, a haloacetamide, haloalkyl, a hydrazine, an isothiocyanate, a maleimide group, an aliphatic amine, a silyl halide, a cadaverine or a psoralen. More preferably, the reactive group is a succinimidyl ester of a carboxylic acid, a maleimide, an iodoacetamide, or a silyl halide. In a particular embodiment the reactive group is a succinimidyl ester of a carboxylic acid, a sulfonyl halide, a tetrafluorophenyl ester, an iosothiocyanates or a maleimide.

In another exemplary embodiment, the present compound is covalently bound to a carrier molecule. If the compound has a reactive group, then the carrier molecule can alternatively be linked to the compound through the reactive group. The reactive group may contain both a reactive functional moiety and a linker, or only the reactive functional moiety.

A variety of carrier molecules are useful in the present invention. Exemplary carrier molecules include antigens, steroids, vitamins, drugs, haptens, metabolites, toxins, environmental pollutants, amino acids, peptides, proteins, nucleic acids, nucleic acid polymers, carbohydrates, lipids, and polymers. In exemplary embodiment, at least one member selected from A, L or B comprises a carrier molecule. Preferably, at least one of A or L comprises a carrier molecule. In another aspect, B comprises a carrier molecule.

In an exemplary embodiment, the carrier molecule comprises an amino acid, a peptide, a protein, a polysaccharide, a nucleoside, a nucleotide, an oligonucleotide, a nucleic acid, a hapten, a psoralen, a drug, a hormone, a lipid, a lipid assembly, a synthetic polymer, a polymeric microparticle, a biological cell, a virus and combinations thereof. In another exemplary embodiment, the carrier molecule is selected from a hapten, a nucleotide, an oligonucleotide, a nucleic acid polymer, a protein, a peptide or a polysaccharide. In a preferred embodiment the carrier molecule is amino acid, a peptide, a protein, a polysaccharide, a nucleoside, a nucleotide, an oligonucleotide, a nucleic acid, a hapten, a psoralen, a drug, a hormone, a lipid, a lipid assembly, a tyramine, a synthetic polymer, a polymeric microparticle, a biological cell, cellular components, an ion chelating moiety, an enzymatic substrate or a virus. In another preferred embodiment, the carrier molecule is an antibody or fragment thereof, an antigen, an avidin or streptavidin, a biotin, a dextran, an IgG binding protein, a fluorescent protein, agarose, and a non-biological microparticle.

In an exemplary embodiment, the enzymatic substrate is selected from an amino acid, peptide, sugar, alcohol, alkanoic acid, 4-guanidinobenzoic acid, nucleic acid, lipid, sulfate, phosphate, —CH₂OCOalkyl and combinations thereof. Thus, the enzyme substrates can be cleave by enzymes selected from the group consisting of peptidase, phosphatase, glycosidase, dealkylase, esterase, guanidinobenzotase, sulfatase, lipase, peroxidase, histone deacetylase, endoglycoceramidase, exonuclease, reductase and endonuclease.

In another exemplary embodiment, the carrier molecule is an amino acid (including those that are protected or are substituted by phosphates, carbohydrates, or C₁ to C₂₂ carboxylic acids), or a polymer of amino acids such as a peptide or protein. In a related embodiment, the carrier molecule contains at least five amino acids, more preferably 5 to 36 amino acids. Exemplary peptides include, but are not limited to, neuropeptides, cytokines, toxins, protease substrates, and protein kinase substrates. Other exemplary peptides may function as organelle localization peptides, that is, peptides that serve to target the conjugated compound for localization within a particular cellular substructure by cellular transport mechanisms. Preferred protein carrier molecules include enzymes, antibodies, lectins, glycoproteins, histones, albumins, lipoproteins, avidin, streptavidin, protein A, protein G, phycobiliproteins and other fluorescent proteins, hormones, toxins and growth factors. Typically, the protein carrier molecule is an antibody, an antibody fragment, avidin, streptavidin, a toxin, a lectin, or a growth factor. Exemplary haptens include biotin, digoxigenin and fluorophores.

In another exemplary embodiment, the carrier molecule comprises a nucleic acid base, nucleoside, nucleotide or a nucleic acid polymer, optionally containing an additional linker or spacer for attachment of a fluorophore or other ligand, such as an alkynyl linkage (U.S. Pat. No. 5,047,519), an aminoallyl linkage (U.S. Pat. No. 4,711,955) or other linkage. In another exemplary embodiment, the nucleotide carrier molecule is a nucleoside or a deoxynucleoside or a dideoxynucleoside.

Exemplary nucleic acid polymer carrier molecules are single- or multi-stranded, natural or synthetic DNA or RNA oligonucleotides, or DNA/RNA hybrids, or incorporating an unusual linker such as morpholine derivatized phosphates (AntiVirals, Inc., Corvallis Oreg.), or peptide nucleic acids such as N-(2-aminoethyl)glycine units, where the nucleic acid contains fewer than 50 nucleotides, more typically fewer than 25 nucleotides.

In another exemplary embodiment, the carrier molecule comprises a carbohydrate or polyol that is typically a polysaccharide, such as dextran, FICOLL, heparin, glycogen, amylopectin, mannan, inulin, starch, agarose and cellulose, or is a polymer such as a poly(ethylene glycol). In a related embodiment, the polysaccharide carrier molecule includes dextran, agarose or FICOLL.

In another exemplary embodiment, the carrier molecule comprises a lipid (typically having 6-25 carbons), including glycolipids, phospholipids, and sphingolipids. Alternatively, the carrier molecule comprises a lipid vesicle, such as a liposome, or is a lipoprotein (see below). Some lipophilic substituents are useful for facilitating transport of the conjugated dye into cells or cellular organelles.

Alternatively, the carrier molecule is a cell, cellular systems, cellular fragment, or subcellular particles, including virus particles, bacterial particles, virus components, biological cells (such as animal cells, plant cells, bacteria, or yeast), or cellular components. Examples of cellular components that are useful as carrier molecules include lysosomes, endosomes, cytoplasm, nuclei, histones, mitochondria, Golgi apparatus, endoplasmic reticulum and vacuoles.

In another exemplary embodiment, the carrier molecule non-covalently associates with organic or inorganic materials. Exemplary embodiments of the carrier molecule that possess a lipophilic substituent can be used to target lipid assemblies such as biological membranes or liposomes by non-covalent incorporation of the dye compound within the membrane, e.g., for use as probes for membrane structure or for incorporation in liposomes, lipoproteins, films, plastics, lipophilic microspheres or similar materials.

In an exemplary embodiment, the carrier molecule comprises a specific binding pair member wherein the present compounds are conjugated to a specific binding pair member and are used to detect nucleic acids. Alternatively, the presence of the labeled specific binding pair member indicates the location of the complementary member of that specific binding pair; each specific binding pair member having an area on the surface or in a cavity which specifically binds to, and is complementary with, a particular spatial and polar organization of the other. Exemplary binding pairs are set forth in Table 2.

TABLE 2
Representative Specific Binding Pairs

| antigen | antibody |
|---|---|
| biotin | avidin (or streptavidin or anti-biotin) |
| IgG* | protein A or protein G |
| drug | drug receptor |
| folate | folate binding protein |
| toxin | toxin receptor |
| carbohydrate | lectin or carbohydrate receptor |
| peptide | peptide receptor |
| protein | protein receptor |
| enzyme substrate | enzyme |
| DNA (RNA) | cDNA (cRNA)† |
| hormone | hormone receptor |
| ion | chelator |

*IgG is an immunoglobulin
†cDNA and cRNA are the complementary strands used for hybridization

In an exemplary embodiment, the present compounds of the invention are covalently bonded to a solid support. The solid support may be attached to the compound either through the A, L or B moiety, or through a reactive group, if present, or through a carrier molecule, if present. Even if a reactive group and/or a carrier molecule are present, the solid support may be attached through the A, L or B moiety. In exemplary embodiment, at least one member selected from A, L or B comprises a solid support. Preferably, at least one of A or L comprises a solid support. In another aspect, L comprises a solid support.

A solid support suitable for use in the present invention is typically substantially insoluble in liquid phases. Solid supports of the current invention are not limited to a specific type of support. Rather, a large number of supports are available and are known to one of ordinary skill in the art. Thus, useful solid supports include solid and semi-solid matrixes, such as aerogels and hydrogels, resins, beads, biochips (including thin film coated biochips), microfluidic chip, a silicon chip, multi-well plates (also referred to as microtitre plates or microplates), membranes, conducting and nonconducting metals, glass (including microscope slides) and magnetic supports. More specific examples of useful solid supports include silica gels, polymeric membranes, particles, derivatized plastic films, glass beads, cotton, plastic beads, alumina gels, polysaccharides such as Sepharose, poly(acrylate), polystyrene, poly(acrylamide), polyol, agarose, agar, cellulose, dextran, starch, FICOLL, heparin, glycogen, amylopectin, mannan, inulin, nitrocellulose, diazocellulose, polyvinylchloride, polypropylene, polyethylene (including poly(ethylene glycol)), nylon, latex bead, magnetic bead, paramagnetic bead, superparamagnetic bead, starch and the like.

In some embodiments, the solid support may include a solid support reactive functional group, including, but not limited to, hydroxyl, carboxyl, amino, thiol, aldehyde, halogen, nitro, cyano, amido, urea, carbonate, carbamate, isocyanate, sulfone, sulfonate, sulfonamide, sulfoxide, etc., for attaching the compounds of the invention. Useful reactive groups are disclosed above and are equally applicable to the solid support reactive functional groups herein.

A suitable solid phase support can be selected on the basis of desired end use and suitability for various synthetic protocols. For example, where amide bond formation is desirable to attach the compounds of the invention to the solid support, resins generally useful in peptide synthesis may be employed, such as polystyrene (e.g., PAM-resin obtained from Bachem Inc., Peninsula Laboratories, etc.), POLYHIPE™ resin (obtained from Aminotech, Canada), polyamide resin (obtained from Peninsula Laboratories), polystyrene resin grafted with polyethylene glycol (TentaGel™, Rapp Polymere, Tubingen, Germany), polydimethyl-acrylamide resin (available from Milligen/Biosearch, California), or PEGA beads (obtained from Polymer Laboratories).

### 5. Preparation of Conjugates

In one embodiment conjugates of components (binding domains, carrier molecules or solid supports), e.g., drugs, peptides, toxins, nucleotides, phospholipids, metal chelating moiety and other organic molecules are prepared by organic synthesis methods using the reactive reporter molecules of the invention, are generally prepared by means well recognized in the art (Haugland, MOLECULAR PROBES HANDBOOK, supra, (2002)). Preferably, conjugation to form a covalent bond consists of simply mixing the reactive compounds of the present invention in a suitable solvent in which both the reactive compound and the substance to be conjugated are soluble. The reaction preferably proceeds spontaneously without added reagents at room temperature or below. For those reactive compounds that are photoactivated, conjugation is facilitated by illumination of the reaction mixture to activate the reactive compound. Chemical modification of water-insoluble substances, so that a desired compound-conjugate may be prepared, is preferably performed in an aprotic solvent such as dimethylformamide, dimethylsulfoxide, acetone, ethyl acetate, toluene, or chloroform. Similar modification of water-soluble materials is readily accomplished through the use of the instant reactive compounds to make them more readily soluble in organic solvents.

Preparation of Peptide or Protein Conjugates Typically Comprises First Dissolving the Protein to be conjugated in aqueous buffer at about. 1-10 mg/mL at room temperature or below. Bicarbonate buffers (pH about 8.3) are especially suitable for reaction with succinimidyl esters, phosphate buffers (pH about 7.2-8) for reaction with thiol-reactive functional groups and carbonate or borate buffers (pH about 9) for reaction with isothiocyanates and dichlorotriazines. The appropriate reactive compound is then dissolved in an aprotic solvent (usually DMSO or DMF) in an amount sufficient to give a suitable degree of labeling when added to a solution of the protein to be conjugated. The appropriate amount of compound for any protein or other component is conveniently predetermined by experimentation in which variable amounts of the compound are added to the protein, the conjugate is chromatographically purified to separate unconjugated compound and the compound-protein conjugate is tested in its desired application.

Following addition of the reactive compound to the component solution, the mixture is incubated for a suitable period (typically about 1 hour at room temperature to several hours on ice), the excess compound is removed by gel filtration, dialysis, HPLC, adsorption on an ion exchange or hydrophobic polymer or other suitable means. The compound-conjugate is used in solution or lyophilized. In this way, suitable conjugates can be prepared from antibodies, antibody fragments, avidins, lectins, enzymes, proteins A and G, cellular proteins, albumins, histones, growth factors, hormones, and other proteins.

Conjugates of polymers, including biopolymers and other higher molecular weight polymers are typically prepared by means well recognized in the art (for example, Brinkley et al., Bioconjugate Chem., 3: 2 (1992)). In these embodiments, a single type of reactive site may be available, as is typical for polysaccharides) or multiple types of reactive sites (e.g. amines, thiols, alcohols, phenols) may be available, as is typical for proteins. Selectivity of labeling is best obtained by selection of an appropriate reactive dye. For example, modification of thiols with a thiol-selective reagent such as a haloacetamide or maleimide, or modification of amines with an amine-reactive reagent such as an activated ester, acyl azide, isothiocyanate or 3,5-dichloro-2,4,6-triazine. Partial selectivity can also be obtained by careful control of the reaction conditions.

When modifying polymers with the compounds, an excess of compound is typically used, relative to the expected degree of compound substitution. Any residual, unreacted compound or a compound hydrolysis product is typically removed by dialysis, chromatography or precipitation. Presence of residual, unconjugated dye can be detected by thin layer chromatography using a solvent that elutes the dye away from its conjugate. In all cases it is usually preferred that the reagents be kept as concentrated as practical so as to obtain adequate rates of conjugation.

In an exemplary embodiment, the conjugate of the invention is associated with an additional substance, that binds either to the reporter molecule or the conjugated substance (carrier molecule or solid support) through noncovalent interaction. In another exemplary embodiment, the additional substance is an antibody, an enzyme, a hapten, a lectin, a receptor, an oligonucleotide, a nucleic acid, a liposome, or a polymer. The additional substance is optionally used to probe for the location of the dye-conjugate, for example, as a means of enhancing the signal of the dye-conjugate.

### 6. Fluorescent Compound Embodiments

The components of the fluorescent compound having now been described, combination of certain fluorophores, linkers and binding domains are provided to demonstrate the complexity of the fluorescent compounds and their application. While it has been stressed that a wide range of components can be used to make the fluorescent compounds it should also be understood that the individual selection of components to make a particularly useful fluorescent compound requires an understanding of the fluorophores, the linkers, the binding domain and how certain combinations function to selectively bind to affinity tags. Therefore, what follows are selected fluorophores indicating sites of attachment, substituents and preferred linkers along with binding domains. However, the following description is in no way limiting and should not be construed as the only preferred embodiments as many fluorophores with linkers attached are equally as preferred. It is understood that the following compounds comprise the salts, acids, chelated metal ions and lipophilic forms including esters of the compounds, as particular forms are advantageous in certain applications. Compounds that comprise acetyloxy methyl (AM) ester are particularly useful for intracellular labeling of affinity tag containing fusion proteins wherein fluorescent compounds comprising AM ester moieties easily enter cells where the ester is cleaved resulting in terminal acetic acid groups on the fluorescent compound. In this way, newly translated fusion proteins can be detected, in vivo, and monitored to ascertain information about the functional proteome of the cell including discovery of drug targets. The terminal acetic acid groups are typically part of the binding domain but they may be other places on the compound.

The linkers of the present invention can be attached at many positions on the fluorophore resulting in an exponential number of fluorescent compounds contemplated by the present invention. Preferred fluorophores of the fluorescent compounds are cyanine, coumarin, borapolyazaindacene, benzofuran and xanthenes including rhodol, rhodamine, fluorescein and derivatives thereof.

Most preferred fluorophores of the fluorescent compounds are coumarin, rhodamine and borapolyazaindacene. The coumarin fluorophore has the Formula (XII), as shown below, wherein A is NH₂, OR′ or N(R′)₂, R′ is H, an alkyl or an acetic acid binding domain and R⁹-R¹² and R⁸ can be any of the corresponding substituents disclosed in U.S. Pat. Nos. 5,696,157 and 5,830,912, supra. Typical substituents include halogen, lower alkyl, alkoxy and hydrogen.

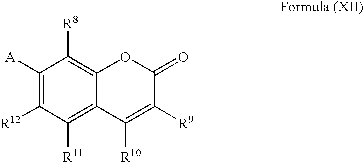

Particularly preferred fluorescent compounds with coumarin as a fluorophore are exemplified in compounds 1, 4, 5 and 6. These exemplified compounds comprise a linker at R⁹ or R¹⁰ having the formula —(CH₂)ₑC(X)NH(CH₂)ₑ(NHC(X)(CH₂)ₑ)_{d}— wherein R⁹ or R¹⁰ that is not a linker is typically a methyl group, R¹¹ is typically hydrogen, R¹² is fluorine (compound 4), sulfonic acid (compound 1) or hydrogen (compound 5 and 6). R¹² is typically hydrogen, however a preferred substituent is fluorine (compound 4). Thus, a preferred compound of the present invention has Formula (VIII) (A)-[(CH₂)ₑC(X)NH(CH₂)ₑ(NHC(X)(CH₂)ₑ)_{d}]ₘ—(B)ₙ wherein A is a coumarin, B is an acetic acid binding domain and d of the linker is typically 0.

Alternatively, the coumarin of Formula (XII) can be any of the compounds disclosed in U.S. Pat. Nos. 5,459,276 and 5,501,980. These compounds comprise a linker at R¹² and the fluorescent compound Formula (XI) (B)(L)(A)-(O)_{d}(CH₂)_{f}O(C₆R″₄)—(B) wherein (B)(L) is A of fluorophore Formula (XII).

It is understood that the linkers of the present invention may be attached at any of R⁸-R¹², and that any of the binding domains of the present invention can be attached to the linker.

The borapolyazaindacene fluorophore has the formula (XIII), as shown below, wherein R¹-R⁷ can be substituted by any of the corresponding substituents disclosed in U.S. Pat. Nos. 5,187,288; 5,248,782 and 5,274,113, supra. Typical substituents include heteroaryl, aryl, lower alky, alkoxy and hydrogen.

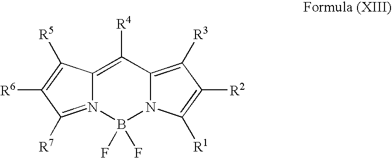

Particularly preferred fluorescent compounds with borapolyazaindacene as a fluorophore are exemplified in compounds 2, 3, 7-14 and 17. These exemplified compounds comprise a linker at R⁷, R⁶, R² or R¹ having the Formula (VI) —((C₆R″₄)O)_{d}(CH₂)ₑ(C(X)NH(CH₂)ₑ)(NH)_{d}C(X)NH(C₆R″₄)(CH₂)ₑ— and/or Formula (V) —(CH₂)ₑC(X)NH(CH₂)ₑ(NHC(X)(CH₂)ₑ)_{d}—, wherein the fluorophore is attached by one linker or two linkers. When the fluorophore is attached by two linkers, the linkers are typically present at R⁶ and R² (Compound 3) or R⁷ and R¹ (Compound 2) and further attached to an acetic acid binding domain. Thus, preferred fluorescent compounds of the present invention have the formula Formula (VIII) (A)-[(CH₂)ₑC(X)NH(CH₂)ₑ(NHC(X)(CH₂)ₑ)_{d}]ₘ—(B)ₙ; Formula (IX) (A)-((C₆R″₄)O)_{d}(CH₂)ₑ(C(X)NH(CH₂)ₑ)(NH)_{d}C(X)NH(C₆R″₄)(CH₂)ₑ—(B), wherein A is borapolyazaindacene and B is an acetic acid binding domain having Formula (I) ⁻O₂CCH(R)N(CH₂CO₂⁻)₂, or formula (III) (CH₂CO₂⁻)_{Z}N[(CH₂)_{S}N(CH₂CO₂⁻)]_{R}(CH₂)ₛN(CH₂CO₂⁻)_{Z}.

Fluorescent compounds comprising acetic acid binding domain Formula (III) can also be used to calorimetrically detect poly-histidine affinity tag containing fusion proteins with the same sensitivity as the fluorescent signal.

The linker and non-linker substituents of the borapolyazaindacene can be present at any of R¹-R⁷. The linkers may be the same or different and may be attached to the same or different binding domains. In this way a fluorescent compound may have affinity for one or more different affinity tags.

The benzofuran fluorophore has the Formula (XIV), as shown below, wherein R¹³-R¹⁸ can be any of the corresponding substituents disclosed in U.S. Pat. Nos. 4,603,209 and 4,849,362, supra. Typical non-linker substituents include hydrogen and substituted heteroaryl.

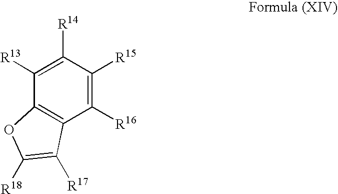

Typical fluorescent compounds comprising a benzofuran fluorophore contain a linker attached at R¹⁴ and R¹⁵, the compounds typically comprise two linkers, one of which is a single covalent bond at R¹⁴ and a linker attached at the R¹⁵ position comprising Linker Formula (VII) —(O)_{d}(CH₂)_{f}O(C₆R″₄)—, such a compound is demonstrated in Compound 12.

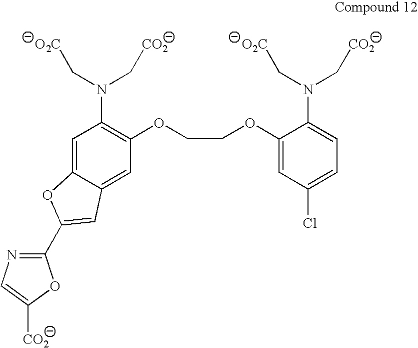

R¹⁸ is typically substituted by a substituted heteroaryl, as Compound 12 demonstrates, preferably an oxazole. Compound 12 also demonstrates a substitution on the benzene ring (C₆R″₄) of the linker; typically the ring is substituted with a halogen, preferably fluorine or chlorine.

The xanthene fluorophore has the Formula (XV), as shown below, wherein F, G and R¹⁹-R²⁵ can be any of the corresponding substituents disclosed in U.S. Pat. Nos. 6,162,931; 6,130,101; 6,229,055; 6,339,392 and 5,451,343, supra. Typically, F is NR′₂ or OR′, G is OR′ or NR′₂, wherein R′ is hydrogen, an alkyl group, linker, an acetic acid binding domain or a linker attached to a binding domain.

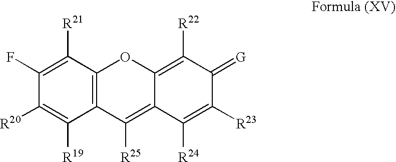

The linkers of the present invention can be present at any of the R groups and with any of the binding domains of the present invention.

When R²⁵ is substituted with a benzene ring (C₆R″₄), as shown below for Formula (XVI), the fluorophore is a rhodol, a rhodamine or a fluorescein depending on F and G. Rhodol fluorophores are represented when F is NR′₂ and G is O, rhodamine fluorophores are represented when F is NR′₂ and G is NR′₂ and fluorescein fluorophores are represented when F is OR′ and G is O. These fluorophores can be substituted by any of the corresponding substituents disclosed in U.S. Pat. Nos. 5,227,487; 5,442,045; 5,798,276; 5,846,737; 6,162,931; 6,130,101; 6,229,055; 6,339,392 and 5,451,343, supra. Fluorescent compounds comprising a xanthene fluorophore are exemplified by rhodamine in Compound 18. In another aspect, the compound does not contain a sulfo group.

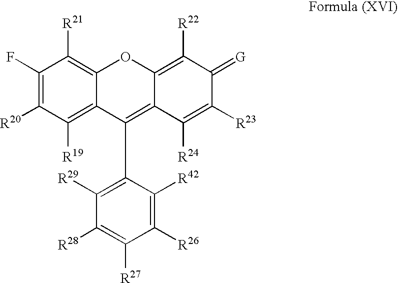

The cyanine fluorophore has the Formula (XVII), as seen below, wherein R³¹-R⁴⁰ and R^{31′}-R^{40′} can be substituted by any of the corresponding substituents disclosed in the U.S. Ser. Nos. 09/968/401 and 09/969,853 and U.S. Pat. Nos. 6,403,807; 6,348,599; 5,486,616; 5,268,486; 5,569,587; 5,569,766; 5,627,027 and 6,048,982, supra. In addition the linkers of the present invention can be substituted at any of the R groups, preferably R⁴⁰, R³¹, R^{40′}, R^{31′}, R³⁹ and R^{39′}, and subsequently attached by a binding domain of the present invention.

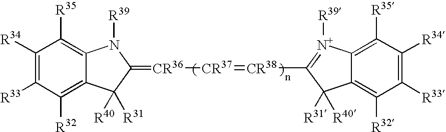

### Methods of Use

The fluorescent compounds of the present invention may be utilized without limit for the site-specific labeling of affinity tags that results in detection of a fusion protein containing a protein of interest and an affinity tag. The methods for detecting a fusion protein containing an affinity tag include contacting a sample with a staining solution and then illuminating the sample whereby the fusion protein is detected.

### Staining Solution

The staining solution comprises 1) an appropriate fluorescent compound that is capable of selectively binding, directly or indirectly, to an affinity tag and 2) a buffer.

Typically, the staining solution comprises a fluorescent compound capable of binding to poly-histidine, poly-arginine, and GST affinity tags wherein the binding domain is selected from the group consisting of glutathione, a positively charged chemical moiety and a negatively charged chemical moiety including acetic acid groups. The fluorophore is selected from the group consisting of xanthene, coumarin, cyanine, acridine, anthracene, benzofuran, indole and borapolyazaindacene.

The staining solution can be prepared in a variety of ways, which is dependent on the medium the sample is in. A particularly preferred staining solution is one that is formulated for detection of affinity tags in a gel. Specifically, the staining solution comprises a fluorescent compound of the present invention in an aqueous solution; optionally the staining solution comprises an organic solvent and a buffering component. The selection of the fluorescent compound dictates, in part, the other components of the staining solution. Any of the components of the staining solution can be added together or separately and in no particular order wherein the resulting staining solution is added to the gel. Alternatively, the components of the staining solution can be added to a gel in a step-wise fashion.

The present invention envisions at least three different versions of a staining solution that comprises a present fluorescent compound. In one embodiment the staining solution comprises a present fluorescent compound, a buffer having a pH about 5 to 6.9, an acceptable counter ion and a metal ion, typically in the form of a salt such as NiCl. In another embodiment, the staining solution comprise a present fluorescent compound, a buffer having a pH about 5 to about 6.9 and acceptable counter ion but without a metal ion such as nickel. In yet another embodiment the staining solution comprises a fluorescent compound pre-loaded with a metal ion, such as nickel, and a buffer having a pH about 7.0 to about 9.

Therefore, in one aspect of the present invention the staining solution for detecting fusion proteins comprising poly-histidine or poly-arginine affinity tags comprises:
a) fluorescent compound having formula A(L)m(B)n wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH about 5 to 6.9 and comprising an acceptable counter ion.

In one aspect this staining solution further comprises nickel ions. In another aspect, this staining solution does not contain nickel ions or any other metal ion. In addition, we have found that in one aspect for the selective detection of poly-histidine affinity tag containing fusion proteins that the buffer preferably contains a salt and has a pKa of about 6.0 to about 7.5. Thus, preferable buffers for this application include Good's buffer, MOPS and PIPES buffers.

The fluorescent compound is prepared by dissolving in a solvent, such as water, DMSO, DMF or methanol, usually at a final concentration of about 0.1 μM to 100 μM, preferably the fluorescent compound is present in the staining solution at a concentration of about 0.2 μM to 20 μM.

Analysis of the selectivity and specificity of the fluorescent compounds for the poly-histidine affinity tags in a SDS-polyacrylamide gel was evaluated as a function of pH. Therefore, a preferred staining solution comprises an acid to provide a moderately acidic environment for the staining reaction. An acidic environment is defined as a solution having a pH less than 6.9. Typical suitable acidic components include without limitation acetic acid, trichloroacetic acid, trifluoroacetic acid, perchloric acid, phosphoric acid, or sulfuric acid. The acidic component is typically present at a concentration of 1%-20%. The pH of the staining mixture is preferably about pH 5-6.9 and most preferred is about pH 6.5. The optimal pH for each compound used may vary slightly depending on the compound used; for compound 1, 2 and 3 pH 6.5 is preferred. Alternatively, a neutral pH is also desirable.

The pH of the staining mixture is optionally modified by the inclusion of a buffering agent in addition to or in place of an acidic component. In particular, the presence of a buffering agent has been shown to improve staining of electrophoresis gels, provided that an alcohol is included in the formulations as well. Any buffering agent that maintains a mild acidic environment and is compatible with the affinity tag and fusion protein in the sample is suitable for inclusion in the staining mixture.

Useful buffering agents include salts of formate, acetate, 2-(N-morphilino) ethanesulfonic acid, imidazole, N-2-hydroxyethyl-piperazine-N′-2-ethanesulfonic acid (PIPES), Tris (hydroxymethyl)aminomethane acetate, or Tris (hydroxymethyl)aminomethane hydrochloride, 3-(N-morpholino) propanesulfonic acid (MOPS). The family of Good's buffers, including TRIS, MES, PIPES, MOPS, are preferred for the present methods. An exemplified buffering agent is PIPES. The buffering agent is typically present in the staining mixture at a concentration of about 10 mM to 500 mM; preferably the concentration is about 25 mM to 100 mM. These buffers are particularly preferred for the non-covalent binding of an acetic acid binding domain to the poly-histidine affinity tag because they have pKa values that are similar to the pKa value of the imidazole ring of the histidine residue.

Optionally, the staining solution may include a polar organic solvent, typically an alcohol, to improve specific staining of the affinity tag. The polar organic solvent, when present, is typically included in the staining solution at a concentration of 5-50%. The presence of a polar organic solvent is particularly advantageous when staining SDS-coated proteins, as is typically the case when staining affinity tags that have been electrophoretically separated on a SDS-polyacrylamide gel. Typically, SDS is removed from a gel prior to staining by fixing, as described below, and washing, however some SDS may remain and interfere with the staining methods of the present invention. Without wishing to be bound by any theory, it appears that the presence of an alcohol improves the affinity of the fluorescent compound for the affinity tag of a fusion protein by removing any SDS that was not removed by the washing or fixing.

Optionally, the staining solution contains a metal ion salt. This is particularly useful for staining solutions used to detect poly-histidine affinity tags and calmodulin affinity tags. Nickel ions and cobalt ions have affinity for both the acetic acid binding domain of the present invention and the poly-histidine affinity tag, therefore nickel or cobalt salts are optionally included in staining solutions of the present invention. While the metal ions do not improve the selective affinity or sensitivity of the binding domain for the poly-histidine affinity tag the inclusion of the metal ions is preferable for certain applications. For this reason, a staining solution to be used to detect poly-histidine affinity tags optionally includes nickel or cobalt ions. An exemplified salt is nickel chloride but any nickel or cobalt salt known to one skilled in the art can be used. The salt is typically present in the staining solution at a concentration of about 10 nm to 1 mM; preferably the concentration is about 1 μM to 200 μM.

Alternatively, some of the compounds of the present invention, especially compounds 7-11, can be used to calorimetrically detect poly-histidine affinity tag containing fusion proteins when the staining solution comprises nickel ions at a concentration about 10 μM. Therefore, a preferred staining solution for calorimetric applications comprises nickel ions at a final concentration of about 10 μM and typically any one of compounds 7-11, See Example 19.

Calcium ions have an affinity for calmodulin, which subsequently alters the conformation of the protein such that it possesses affinity for the calmodulin affinity tag. Therefore, a staining solution specific for calmodulin contains a calcium salt along with a fluorescent compound that contains a fluorophore covalently attached to the calmodulin protein.

In another embodiment of the staining solution, the staining solution contains a fluorescent compound that is pre-complexed with a metal ion, wherein the staining solution comprises:
a) fluorescent compound having formula A(L)m(B)n that has been pre-loaded with nickel ions, wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH about 7.0 to 9.0

In this instance, we have found that aqueous phosphate is an excellent buffer for the present staining solution, although Tris and tricine are equally preferred buffers for maintaining an basic staining solution that facilitates detection of histidine tagged fusion proteins. Due to the pre-loading of nickel on the fluorescent compound the stock solution of dye contains organic solvents resulting in trace amounts in the staining solution. In one embodiment, the fluorescent compound is pre-loaded with nickel by incubating with nickel sulfate wherein the excess nickel is washed away following purification on a reverse phase sep-pak cartridge. This procedure typically results in a 50% acetonitrile/50% water solution wherein the fluorescent compound-nickel complex is present at about 1-10 mM. This concentrate is then diluted in the aqueous phosphate buffer at a final concentration of about 0.1 μM to about 10 μM. The concentration range is altered in part depending on the fluorophore substituents, the number of binding domains and the placement of the binding domains in relation to each other. In a particular embodiment we have found that Compound 18, when pre-complexed with nickel ions, in optimally present in the staining solution at about 0.2 μM with about 20 mM phosphate. All of these parameters can be altered depending on the properties of the specific compound including the presence or absence of pre-complexed metal ions.

This basic staining solution may further comprise any of the additional components described above. In addition, it is contemplated for certain circumstances that the staining solution is has a pH about 7.0 to about 9.0 wherein the fluorescent compound has not been pre-loaded with metal ions but are rather provided as a salt in the buffer. However, we have found an increase in the detection limit because the nickel ions are bound first by the fluorescent compound and then bound by the histidine containing fusion protein driving a 1:1 ternary complex. Providing the nickel ions as part of the buffer allows for nickel ions to bind both the histidine containing fusion protein and fluorescent compound wherein a ternary complex is not formed due to nickel ions in both binding sites.

In one embodiment, the present invention provides methods for detecting histidine-containing fusion proteins that have been immobilized, typically by electrophoresis. In another embodiment, the present invention provides methods for isolation and purification of histidine-containing fusions proteins.

Electrophoresis is a preparative and/or analytical method used to separate and characterize macromolecules. It is based on the principle that charged particles migrate in an applied electrical field. For a review of electrophoretic methods that are used to separate molecules, particularly proteins, see Chiou et al., Analytica Chimica Acta 383:47-60 (1999).

If electrophoresis is carried out in solution, molecules are separated according to their surface net charge density. If carried out in semisolid materials (gels), however, the matrix of the gel adds a sieving effect so that particles migrate according to both charge and size. Protein electrophoresis can performed in the presence of a charged detergent like sodium dodecyl sulfate (SDS) which coats the surface of, and thus equalizes the surface charge of, most proteins, so that migration depends on size (molecular weight). Proteins are often separated in this fashion, i.e., SDS-PAGE (PAGE=polyacrylamide gel electrophoresis). One or more denaturing agents, such as urea, can also be included in order to minimize the effects of secondary and tertiary structure on the electrophoretic mobility of proteins. Such additives are typically not necessary for nucleic acids, which have a similar surface charge irrespective of their size and whose secondary structures are generally broken up by the heating of the gel that happens during electrophoresis.

In general, electrophoresis gels can be either in a slab gel or tube gel form. For slab gels, the apparatus used to prepare them usually consists of two glass or plastic plates with a space disposed between them by means of a spacer or gasket material and the apparatus is held together by a clamping means so that the space created is closed on three sides and open at the top. A solution of unpolymerized gel is poured into the space while in its liquid state. A means of creating wells or depressions in the top of the gel (such as a comb) in which to place samples is then placed in the space. The gel is then polymerized and becomes solid. After polymerization is complete, the comb device is removed and the gel, while still held within the plates, is then ready for use. Examples of such apparatus are well known and are described in U.S. Pat. Nos. 4,337,131 to Vesterberg; 4,339,327 to Tyler; 3,980,540 to Hoefer et al.; 4,142,960 to Hahn et al.; 4,560,459 to Hoefer; and 4,574,040 to Delony et al. Tube gels are produced in a similar manner, however, instead of glass or plastic plates, glass capillary tubing is used to contain the liquid gel.

Two commonly used electrophoretic media for gel electrophoresis and other separation techniques are agarose and polyacrylamide. Each of these is described in turn as follows. In standard PAGE technology, gels commonly range between about 5% to about 22.5% T (T=total amount of acrylamide or other gelling agent), mostly between about 7.5% and about 15% T. Lower percentages may be employed with linear polyacrylamide. In agarose gel electrophoresis, concentrations between about 0.2% and about 2% T may be employed.

Agarose is a colloidal extract prepared from seaweed. Different species of seaweed are used to prepare agarose; commercially available agarose is typically prepared from genera including, but not limited to, Gracilaria, Gelidium, and Pterocladia. It is a linear polysaccharide (average molecular mass of about 12,000) made up of the basic repeat unit agarobiose, which comprises alternating units of galactose and 3,6-anhydrogalactose. Agarose contains no charged groups and is thus useful as a medium for electrophoresis.

Agarose gels have very large “pore” size and are used primarily to separate large molecules, e.g., those with a molecular mass greater than about 200 kilodaltons (kD). Agarose gels can be prepared, electrophoresed (“run”) and processed faster than polyacrylamide gels, but their resolution is generally inferior. For example, for some macromolecules, the bands formed in agarose gels are “fuzzy” (diffuse). The concentration of agarose typically used in gel electrophoresisis is between from about 1% to about 3%.

Agarose gels are formed by suspending dry agarose in an aqueous, usually buffered, media, and boiling the mixture until a clear solution forms. This is poured into a cassette and allowed to cool to room temperature to form a rigid gel.

Acrylamide polymers are used in a wide variety of chromatographic and electrophoretic techniques and are used in capillary electrophoresis. Polyacrylamide is well suited for size fractionation of charged macromolecules such as proteins and nucleic acids (e.g., deoxyribonucleic acids, a.k.a. DNA, and ribonucleic acids, a.k.a. RNA).

The creation of the polyacrylamide matrix is based upon the polymerization of acrylamide in the presence of a crosslinker, usually methylenebisacrylamide (bis, or MBA). Upon the introduction of catalyst, the polymerization of acrylamide and methylene bisacrylamide proceeds via a free-radical mechanism. The most common system of catalytic initiation involves the production of free oxygen radicals by ammonium persulfate (APS) in the presence of the tertiary aliphatic amine N,N,N′,N′-tetramethylethylenediamine (TEMED). Various other chemical polymerization systems may be used. For example, TEMED and persulfate may be added to provide polymerization initiation. If desired, an acrylamide gradient may be developed by successively adding solutions with increasing amounts of acrylamide and/or cross-linking agent. Alternatively, differential initiation may be used, so as to provide varying degrees of polymerization and thus prepare a gradient gel.

Electrophoretic gels based on polyacrylamide are produced by co-polymerization of monoolefinic monomers with di- or polyolefinic monomers. The co-polymerization with di- or polyfunctional monomers results in cross-linking of the polymer chains and thereby the formation of the polymer network. Monoolefinic monomers include, by way of non-limiting example, acrylamide, methacrylamide and derivatives thereof such as alkyl-, or hydroxyalkyl derivates, e.g., N-hydroxymethylacrylamide, N,N-dimethylacrylamide, N-hydroxypropylacrylamide. The di- or polyolefinic monomer is preferably a compound containing two or more acryl or methacryl groups such as e.g. methylenebisacrylamide, N,N′-diallyltartardiamide, N,N′-1,2-dihydroxyethylene-bisacrylamide, N,N-bisacrylyl cystamine, trisacryloyl-hexahydrotriazine. In a broader sense, polyacrylamide also includes gels in which the monoolefinic monomer is selected from acrylic- and methacrylic acid derivatives, e.g., alkyl esters such as ethyl acrylate and hydroxyalkyl esters such as 2-hydroxyethyl methacrylate, and in which cross-linking has been brought about by means of a compound as mentioned before. Further examples of gels based on polyacrylamide are gels made by co-polymerization of acrylamide with a polysaccharide substituted to contain vinyl groups such as allyl glycidyl dextran (see EP 0 087 995).

One type of electrophoresis is usually referred to as isoelectric focusing (IEF) or electrofocusing. IEF, which can be carried out in an electrophoretic medium or in solution, involves passing a mixture through a separation medium which contains, or which may be made to contain, a pH gradient or other pH function. The device or gel has a relatively low pH at one end, while at the other end it has a higher pH. IEF is discussed in various texts such as Isoelectric Focusing by P. G. Righetti and J. W. Drysdale (North Holland Publ., Amsterdam, and American Elsevier Publ., New York, 1976).

The charge on a protein or other molecule depends on the pH of the ambient solution. At the isoelectric point (pl) for a certain molecule, the net charge on that molecule is zero. At a pH above its pl, the molecule has a negative charge, while at a pH below its pl the molecule has a positive charge. Each different molecule has a characteristic isoelectric point. When a mixture of molecules is electrophoresed in an IEF system, an anode (positively charged) is placed at the acidic end of the system, and a cathode (negatively charged) is placed at the basic (alkaline) end. Each molecule having a net positive charge under the acidic conditions near the anode will be driven away from the anode. As they electrophorese through the IEF system, molecules enter zones having less acidity, and their positive charges decrease. Each molecule will stop moving when it reaches its particular pl, since it no longer has any net charge at that particular pH. This effectively separates molecules that have different pl values. The isolated molecules of interest can be removed from the IEF device by various means, or they can be stained or otherwise characterized.

Some types of IEF systems generate pH gradients by means of “carrier ampholytes.” These are synthetic ampholytes that often have a significant amount of buffering capacity. When placed in an IEF device, each carrier ampholyte will seek its own isoelectric point. Because of their buffering capacity, many carrier ampholytes will establish a pH plateau rather than a single point. By using a proper mixture of carrier ampholytes, it is possible to generate a relatively smooth pH gradient for a limited period of time. Such mixtures are sold commercially under various trade names, such as Ampholine (sold by LKB-Produkter AB of Bromma, Sweden), Servalyt (sold by Serva Feinbiochemica of Heidelberg, FRG), and Pharmalyte (sold by Pharmacia Fine Chemicals AB, Uppsala, Sweden). The chemistry of ampholyte mixtures is discussed in various references, such as U.S. Pat. No. 3,485,736; Matsui et al., Methods Mol. Biol. 112:211-219 (1999); and Lopez, Methods Mol. Biol. 112:109-110 (1999).

In IEF in Immobilized pH gradients (IPG), amphoretic ions are forced to reach a steady-state position along pH inclines of various scopes and spans (see Righetti et al., Electrophoresis 15:1040-1043, 1994; Righetti et al., Methods Enzymol. 270:235-255, 1996; and 2-D Electrophoresis using immobilized pH gradients—Principles and Methods, Edition AC, Berkelman, T. and T. Stenstedt, Amersham Biosciences, Freiburg, Germany, 1998.). In one popular version of IPG, the pH gradient is in the form of a strip and is referred to as a “strip gel” or a “gel strip” that can be used in appropriate formats. See, by way of non-limiting example, published PCT patent applications WO 98/57161 A1, WO 02/09220 A1, published U.S. patent application US 2003/0015426 A1, and U.S. Pat. Nos. 6,599,410; 6,156,182; 6,113,766; and 6,495,017.

Two dimensional (2D) electrophoresis techniques are also known, involving a first electrophoretic separation in a first dimension, followed by a second electrophoretic separation in a second, transverse dimension. In the 2D method most commonly used, proteins are subjected to IEF in a polyacrylamide gel in the first dimension, resulting in separation on the basis of isolectric point (pl), and are then subjected to SDS-PAGE in the second dimension, resulting in further separation on the basis of size (O'Farrell, J. Biol. Chem. 250:4007-4021, 1975).

Electrophoresis also includes techniques known collectively as capillary electrophoresis (CE). Capillary electrophoresis (CE) achieves molecular separations on the same basis as conventional electrophoretic methods, but does so within the environment of a narrow capillary tube (25 to 50 μm). The main advantages of CE are that very small (nanoliter) volumes of sample are required; moreover, in a capillary format, separation and detection can be performed rapidly, thus greatly increasing sample throughput relative to gel electrophoresis. Some non-limiting examples of CE include capillary electrophoresis isoelectric focusing (CE-IEF) and capillary zone electrophoresis (CZE).

Capillary zone electrophoresis (CZE) is a technique that separates molecules on the basis of differences in mass to charge ratios, which permits rapid and efficient separations of charged substances (for a review, see Dolnik, Electrophoresis 18:2353-2361, 1997). In general, CZE involves introduction of a sample into a capillary tube, i.e., a tube having an internal diameter from about 5 to about 2000 microns, and the application of an electric field to the tube. The electric potential of the field both pulls the sample through the tube and separates it into its constituent parts. Each constituent of the sample has its own individual electrophoretic mobility; those having greater mobility travel through the capillary tube faster than those with slower mobility. As a result, the constituents of the sample are resolved into discrete zones in the capillary tube during their migration through the tube. An on-line detector can be used to continuously monitor the separation and provide data as to the various constituents based upon the discrete zones.

CZE can be generally separated into two categories based upon the contents of the capillary columns. In “gel” CZE, the capillary tube is filled with a suitable gel, e.g., polyacrylamide gel. Separation of the constituents in the sample is predicated in part by the size and charge of the constituents traveling through the gel matrix. This technique, sometimes referred at as capillary Gel Electrophoresis (CGE), is described by Hjertñ\ddot{l} (J. Chromatogr. 270:1, 1983), and is suitable for resolving macromolecules that differ in size but have a constant charge-to-mass ratio (Guttman et al., Anal. Chem. 62:137, 1990).

In “open” CZE, the capillary tube is filled with an electrically conductive buffer solution. Upon ionization of the capillary, the negatively charged capillary wall will attract a layer of positive ions from the buffer. As these ions flow towards the cathode, under the influence of the electrical potential, the bulk solution (the buffer solution and the sample being analyzed), must also flow in this direction to maintain electroneutrality. This electroendosmatic flow provides a fixed velocity component, which drives both neutral species and ionic species, regardless of charge, towards the cathode. Fused silica is principally utilized as the material for the capillary tube because it can withstand the relatively high voltage used in CZE, and because the inner walls of a fused silica capillary ionize to create the negative charge which causes the desired electroendosomatic flow. The inner wall of the capillaries used in CZE can be either coated or uncoated. The coatings used are varied and known to those in the art. Generally, such coatings are utilized in order to reduce adsorption of the charged constituent species to the charged inner wall. Similarly, uncoated columns can be used. In order to prevent such adsorption, the pH of the running buffer, or the components within the buffer, are manipulated.

The gel-based electrophoretic embodiments of the invention can be carried out in any suitable format, e.g., in standard-sized gels, minigels, strips, gels designed for use with microtiter plates and other high throughput (HTS) applications, and the like. Minigel and other formats include without limitation those described in the following patents and published patent applications: U.S. Pat. No. 5,578,180, to Engelhorn et al., entitled “System for pH-Neutral Longlife Electrophoresis Gel”; U.S. Pat. Nos. 5,922,185; 6,059,948; 6,096,182; 6,143,154; 6,162,338, all to Updyke et al.; published U.S. Patent Applications 20030127330 A1 and 20030121784 A1; and published PCT Application WO 95/27197, all entitled “System for pH-Neutral Stable Electrophoresis Gel”; U.S. Pat. No. 6,057,106, to Updyke et al., and published PCT application WO 99/37813, both entitled “Sample Buffer and Methods for High Resolution Gel Electrophoresis of Denatured Nucleic Acids”; U.S. Pat. No. 6,562,213 to Cabilly et al., and published PCT application WO 02/18901, both entitled “Electrophoresis Apparatus for Simultaneous Loading of Multiple Samples”; and published U.S. Patent Application 2002/0134680 A1, to Cabilly et al., and published PCT application WO 02/071024, both entitled “Apparatus and Method for Electrophoresis”.

Any suitable buffer can be used to practice the electrophoretic modalities of the invention. Non-limiting examples of buffers include those described herein and in the preceding patents and published patent applications, as well as those described in Righetti et al., Electrophoresis 15:1040-1043 (1994); Chiari et al., Appl Theor Electrophor. 1:99-102 (1989); and Chiari et al., Appl Theor Electrophor. 1:103-107 (1989).

In addition, after proteins have been separated and immobilized in a polymeric gel, the proteins can be further transferred and immobilized on a polymeric membrane prior to detection. Such membranes include, but are not limited to nitrocellulose and PVDF.

Therefore, the present invention provides a staining solution and methods for detection affinity tag-containing fusion proteins. An example of an appropriate matching of a fluorescent compound and affinity tag is a poly-histidine affinity tag and a fluorescent compound that contains an acetic acid binding domain. The acetic acid binding domain is capable of selectively interacting with either a metal ion or the positively charged poly-histidine affinity tag. Thus, in one aspect of the invention specific fluorescent compounds are used to detect and label fusion proteins that contain a poly-histidine affinity tag. A method of the present invention wherein the poly-histidine affinity tag containing fusion protein is detected after being separated on a polyacrylamide gel comprises the following steps:
i) immobilizing the sample on a solid or semi-solid matrix to prepare an immobilized sample;
ii) optionally contacting the immobilized sample with a fixing solution to prepare a fixed sample;
iii) contacting the immobilized sample of with a staining solution comprising a fluorescent compound capable of selectively binding to a poly-histidine affinity tag to prepare a staining sample;
iv) incubating the stained sample for a sufficient amount of time to allow the fluorescent compound to associate with the poly-histidine affinity tag to prepare an incubated sample;
v) illuminating the incubated sample with a suitable light source to prepare an illuminated sample; and
vi) observing the illuminated sample whereby the fusion protein is detected.

In step one (1) a sample, obtained as described below, is prepared in an appropriate buffer and immobilized on a solid or semi-solid matrix. Typically the sample is separated on a gel, typically a SDS-polyacrylamide gel. Alternatively, the sample is immobilized on solid or semi-solid matrix that includes a membrane, polymeric beads, polymeric gel, a glass surface or an array surface. When SDS-polyacrylamide gels are employed, the denaturing effects of the SDS buffer allow for the exposure of the affinity tag because when folded into a native form the affinity tag can be obscured from compounds that have affinity for the peptide. Thus, SDS gel electrophoresis facilitates the binding of the fluorescent compounds of the present invention with the poly-histidine affinity tag of a fusion protein. However, after the sample has been separated it is important that the SDS be removed from the gel with a fixing solution for maxima detection of the affinity tag because the SDS interferes with the affinity of the fluorescent compound for the affinity tag.

Therefore, the second (2) step optionally comprises incubating the gel in a fixing solution that typically includes an alcohol so as to remove the SDS before the staining solution is added to the gel. Typically, effective removal of SDS requires a step-wise contact with the fixing solution wherein the fixing solution is incubated with the gel, removed and new solution is added for an additional time period. Following the fixing step, the gel is typically rinsed with water.

During the third (3) and fourth (4) steps, the gel containing the sample, is contacted with a staining solution for a time period that permits effective non-covalent labeling of the fluorescent compound to the affinity tag. Typically this time period is from about 30 minutes to about 120 minutes. The staining solution contains a fluorescent compound that is capable of directly or indirectly binding to the affinity tag of the fusion protein and has the general formula A(L)m(B)n, as described above. For the binding of poly-histidine affinity tags, fluorescent compounds that contain an acetic acid-binding domain are preferred. Exemplified compounds 1-16 are particularly preferred. The staining solution optionally comprises an appropriate metal ion, an appropriate metal ion being one that has affinity for both the fluorescent compound and the affinity tag. As described above, some of the affinity tags have an affinity for metal ions, therefore for particular applications; a metal ion is desirable in the staining solution. The staining solution may be pre-mixed and added to the gel in one step or the individual components may be added step-wise to the gel. Preferably the gel is subjected to mild agitation while in contact with the staining solution.

During the fifth (5) and sixth (6) steps, the gel is illuminated and observed with a suitable light source that allows for the fluorophore of the fluorescent compound affinity tag complex to be visualized whereby the fusion proteins containing a poly-histidine affinity tag is detected. Preferably, the gel is rinsed with water to remove unbound fluorescent compound prior to illumination. The suitable light source is dictated by the fluorophore of the fluorescent compound. For example, a staining solution comprising compound 1 exhibits bright-blue fluorescence (emission maximum=450 nm) when illuminated with UV-A or UV-B light from a standard ultraviolet transilluminator and compound 2 exhibits bright-green fluorescence (emission maximum=515 nm) when illuminated with visible light from a laser-based gel scanner equipped with a 470 nm second-harmonic generation (SHG) or 488 nm argon ion laser source. Typically, detection limits of poly-histidine affinity tag containing fusion proteins using staining solution containing Compound 1 or 2 is 25-65 ng in whole cell lysates.

### Fixing Solution

The fixing solution is required for optimal staining of poly-histidine affinity tags that have been separated and immobilized in an SDS-polyacrylamide gel. When fusion proteins are denatured and separated on a polyacrylamide gel they become coated with SDS, which masks the affinity tag such that the fluorescent compound will not specifically or selectively bind to the affinity tag. Therefore, the SDS must be removed prior to addition of the staining solution.

The fixing solution contains a polar organic solvent, typically an alcohol. Preferably, the polar organic solvent is an alcohol having 1-6 carbon atoms, or a diol or triol having 2-6 carbon atoms. Preferred alcohols are methanol or ethanol mixed with acetic acid. The alcohols are present in an aqueous solution of about 50% ethanol or methanol with 10% acetic acid. Fixing solutions containing less than 50% of ethanol or methanol generally result in incomplete removal of SDS from the gels.

To remove the SDS coat from the immobilized fusion proteins, the polyacrylamide gel is incubated in the fixing solution. Preferably the gel is fixed in multiple sequential steps, typically two. Essentially, the gel is immersed in the fixing solution for at least 20 minutes and then removed from the solution and new solution added for at least 3 hours and up to 24 hours. Generally, one step of incubating the gel in fixing solution is insufficient to remove all the SDS from the gel.

### Sample Preparation

The fusion proteins of the invention can be expressed in a number of systems including genetically engineered animals or plants, or in cells such as bacteria, yeast, insect, plant and mammalian cell cultures. The preparation of fusion proteins comprising an affinity tag can be made using standard recombinant DNA methods. Typically, a protein of interest, which is determined by the end user, is synthesized and inserted into a vector containing an affinity tag such that when inserted in frame the affinity tag and protein of interest will be translated as one fusion protein. There are many vectors that are available to one skilled in the art that contain nucleotide sequence for an affinity tag, such as pGEX (Amersham Biosciences) for GST affinity tag, pCAL (Stratagene) for calmodulin affinity tag, pFLAG (Sigma Aldrich) for FLAG affinity tag, 6× his tag vector (Qiagen) for poly-histidine affinity tag and expression vectors for Glu-Glu affinity tag including many expression systems available from Invitrogen containing vectors with a combination of affinity tags (U.S. Pat. No. 6,270,969). Alternatively, a nucleotide sequence coding for a desired affinity tag is first synthesized and then linked to a nucleotide sequence coding for the protein of interest. This fused polynucleotide is then inserted into an expression vector using techniques well known to those skilled in the art, wherein the fusion protein will be expressed when the vector is induced in a host cell such as E. coli. (Maniatis et al. “Molecular Cloning” (2002), Cold Spring Harbor Laboratory).

Expression systems for expressing the fusion proteins are available using E. coli, Bacillus sp. (Palva, I. et al., (1983) Gene 22:229-235; Mosbach, K. et al., (1983) Nature 302:543-545) Yeast and Salmonella. The polynucleotides encoding the fusion proteins can also be ligated to various expression vectors for use in transforming mammalian or insect cell cultures. Illustrative examples of mammalian cell lines include VERO, COS, and HeLa cells, Chinese hamster ovary (CHO) cell lines, and various cell lines available from American Type Culture Collection (Bethesda, Md.). Suitable insect cell lines include mosquito larvae, silkworm, armyworm, moth and Drosophila cell lines.

Expression and isolation of fusion proteins are also well known in the art (Maniatis et al, supra). Essentially, a suitable host organism is transformed with an expression vector in which the protein of interest or fused polynucleotide described above is operably linked to an expression control sequence. The transformed host cells are grown under suitable growth conditions wherein the expression vector is induced to produce fusion proteins. When the fusion protein is secreted out of the host organism the cell culture media is collected and the soluble proteins are concentrated. Alternatively, when the fusion protein is an intracellular protein the host cells are pelleted and using standard techniques the proteins are extracted wherein preferably the DNA and lipids of the cell are removed from the crude cellular extract.

When the sample is to be separated on a SDS-polyacrylamide gel the sample is first equilibrated in an appropriate buffer, such as a SDS-sample buffer containing Tris, glycerol, DTT, SDS, and bromophenol blue.

Alternatively, the constructs encoding the fusion protein of the invention are used to produce a genetically engineered animal or plant. For production of genetically engineered animals (e.g., mice, rats, guinea pigs, rabbits, and the like) the construct can be introduced into cells in vitro or in vivo. These nucleic acids can be inserted into any of a number of well-known vectors for the transfection of target cells and organisms.

After expression in the genetically engineered animal, the fusion protein is detected in a sample from the animal. The sample can be a biological fluid such as whole blood, plasma, serum, nasal secretions, sputum, saliva, urine, sweat, transdermal exudates, cerebrospinal fluid, or the like. Alternatively, the sample may be whole organs, tissue or cells from the animal. Examples of sources of such samples include muscle, eye, skin, gonads, lymph nodes, heart, brain, lung, liver, kidney, spleen, solid tumors, macrophages, mesothelium, and the like. In addition, the fusion protein may be detected intracellularly wherein a live-cell version of the present fluorescent compounds are used.

### Illumination

At any time after staining and during the washing step, the sample is illuminated with a wavelength of light selected to give a detectable optical response, and observed with a means for detecting the optical response. Equipment that is useful for illuminating the fluorescent compounds of the present invention includes, but is not limited to, hand-held ultraviolet lamps, mercury arc lamps, xenon lamps, lasers and laser diodes. These illumination sources are optically integrated into laser scanners, fluorescent microplate readers or standard or microfluorometers. The degree and/or location of staining, compared with a standard or expected response, indicates whether and to what degree the sample possesses a given characteristic, i.e. fusion protein containing an affinity tag.

The optical response is optionally detected by visual inspection, or by use of any of the following devices: CCD camera, video camera, photographic film, laser-scanning devices, fluorometers, photodiodes, quantum counters, epifluorescence microscopes, scanning microscopes, flow cytometers, fluorescence microplate readers, or by means for amplifying the signal such as photomultiplier tubes. Where the sample is examined using a flow cytometer, examination of the sample optionally includes sorting portions of the sample according to their fluorescence response.

### Kits of the Invention

Suitable kits for detecting and selectively and non-covalently labeling an affinity tag of a fusion protein also form part of the invention. Such kits can be prepared from readily available materials and reagents and can come in a variety of embodiments. The contents of the kit will depend on the design of the assay protocol or reagent for detection or measurement. All kits will contain instructions, appropriate reagents and label, and solid supports, as needed. Typically, instructions include a tangible expression describing the reagent concentration or at least one assay method parameter such as the relative amounts of reagent and sample to be added together, maintenance time periods for reagent/sample admixtures, temperature, buffer conditions and the like to allow the user to carry out any one of the methods or preparations described above.

Typically, kits useful for detecting an affinity tag of a fusion protein that has been separated on a SDS-polyacrylamide gel will include a staining solution. The kits will optionally include affinity tag containing molecular weight markers, a fixing solution and an additional detection reagent.

Typically, the affinity tag containing molecular weight markers will be stained by the fluorescent compounds of the present invention and are thus useful for estimating the size of the detected fusion protein. This enables the end user to quickly determine if a full-length fusion protein has been produced based on the estimated molecular weight. A fixing solution, as described above, is useful for removing the SDS from the polyacrylamide gel as some of the compounds of the present invention will have minimal affinity for the affinity tag in the presence of SDS. This is particularly true for the fluorescent compounds that are used for selectively binding to the poly-histidine affinity tag. Alternatively, the end user may supply the fixing solution, as this is made with reagents (alcohol) well known to one skilled in the art.

Typically, an additional detection reagent will include a total protein stain such as SYPRO® Ruby Dye and any corresponding total protein stain disclosed in U.S. Pat. No. 6,316,276. Because SDS is removed by the fixing solution prior to addition of the staining solution of the present invention, total protein stains such as SYPRO Ruby are preferred because SDS is not critical for the staining function. However, protocol changes can be made when using a total protein stain that requires SDS for staining sensitivity, such as SYPRO Orange Dye and SYPRO Red Dye, by adding SDS back to the gel prior to a total protein stain step and including SDS in the staining solution (Malone et al. Electrophoresis (2001) 22(5):919-32). A preferred solution for returning SDS back to a gel is 2% acid/0.0005% SDS, and optionally 40% ethanol, wherein the gel is incubated for at least one hour. Alternatively, the total protein stain could be preformed prior to detection of the affinity tag with the staining solution of the present invention; therefore the SDS would not need to be added back to the gel but simply removed prior to affinity tag detection as contemplated by the present invention. Therefore, alternative preferable total protein stains for gels are SYPRO Orange Dye, SYPRO Tangerine Dye, SYPRO Red Dye, Coomassie Fluor dyes or any corresponding dye disclosed in U.S. Pat. Nos. 5,616,502 and 6,579,718. Alternative total protein stains for gels include Coomassie Blue or silver staining, staining techniques well known to those skilled in the art.

The staining solution of the kit will depend on (1) the affinity tag to be detected and (2) the desired absorption and emission spectra from the fluorescent compound. The choice of the binding domain dictates the particular affinity tag that will be detected. As described above, particular binding domains of the present invention have affinity for poly-histidine affinity tag, poly-arginine affinity tag, GST affinity tag and calmodulin affinity tag. The absorption and emission spectra of the fluorescent compound is dictated by the fluorophore. The fluorophores of the present invention cover almost the entire spectrum of UV light, including the popular wavelengths 488, 532 and 633. Particularly useful fluorophores in fluorescent compounds for detecting poly-histidine affinity tags are coumarin, benzofuran, borapolyazaindacene, cyanine and xanthenes. Another important aspect of the staining solution is the pH and the pKa value wherein the optimal pH is dependent on the fluorescent compound in the staining solution and the pKa value is dependent on the affinity tag. Typically, a staining solution for detecting poly-histidine affinity tags is mildly acidic or neutral, pH 5 to 7, and has a pKa of about 6.0 to about 7.5. Preferred is a pH about 6.5 and a pKa of about 6.8.

It is understood by one skilled in the art, that any of the fluorescent compounds contemplated by the present invention can be used to in a staining solution to be included in a kit. The compounds are not intended to be limited to only the described preferred embodiments.

### Applications

The compounds and methods described above for the site-specific labeling of affinity tags has many applications and is not simply limited to detection of affinity tags on a solid or semi-solid matrix. One skilled in the art will appreciate many other applications the fluorescent compound of the present invention can be used in. For example, the fluorescent compounds may be used to label affinity tag containing fusion protein in a solution. This would serve the purpose for a quick determination for the presence of the desired fusion protein or for more involved applications wherein the fluorescent compound functions as a tracer of the fusion protein in an in vitro assay. Such assays may involve, but are not limited to, the study of protein-protein interaction, signal transduction, post-translational modifications, monitoring, metabolism and cell trafficking.

One skilled in the art will also recognize that live cell (cell permeant) versions of the fluorescent compounds could be used in a wide range of in vivo assays. Affinity tag containing fusion proteins could be produced in an appropriate host cell, eukaryotic or prokaryotic, and the fluorescent compounds of the present invention could site-specifically label the intracellular fusion proteins providing for a rigorous analysis of a protein of interest. One could envision that this would be applicable for determining drug targets or studying the functional proteome.

In one embodiment, modification of carboxylic groups with acetoxymethyl (AM) ester groups results in uncharged molecules than can penetrate cell membranes. Once inside the cells, the lipophilic blocking groups are cleaved by nonspecific esterases revealing a binding domain of the present invention, e.g., acetic acid binding domain.

By way of example, the following present compound (Compound 13) has been derivatized to comprise three AM ester groups.

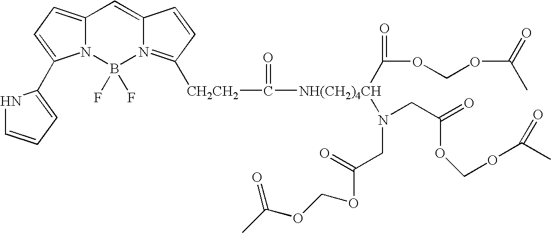

When the compound enters a cell the AM ester groups will be cleaved revealing an acetic acid binding domain according to the following structure (Compound 14).

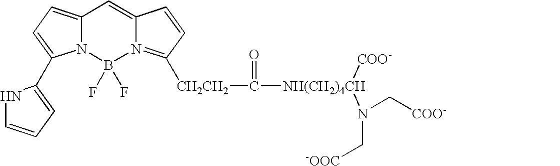

Thus, the present compounds that comprise acetic acid binding domains can be represent by the formula —N(CH₂COOR³⁰) wherein R³⁰ is the same or different and is selected from the group consisting of hydrogen, salt ions, an electron pair and —CH₂OCOCH₃(AM ester). In this way the compounds of the present invention represent both cell permeant and cell impermeant versions wherein for the live cell versions the AM ester is cleaved unmasking the acetic acid binding domain.

Fluorogenic versions of the fluorescent compounds, i.e., version that demonstrate a detectable change upon non-covalently binding to an affinity tag or compounds that are essentially non-fluorescent until bound to an affinity tag, could be used in certain applications. For example, the fluorogenic compounds could be attached to a solid or semi-solid matrix and when an aliquot of a sample thought to contain an affinity tag was added a change in the detectable response would indicate the presence of an affinity tag. Such solid or semi-solid matrix include without limitation, multiwell plastic microplates, glass slides, polymeric particles and arrays.

Additionally, some of the fluorescent compounds are also calorimetric, especially compounds 7-10. These compounds can be used in the same applications as the non-colorimetric compounds however these compounds are especially useful for detecting affinity tags in SDS-polyacrylamide gels and membrane blots. The use of the calorimetric fluorescent compounds can be equally as sensitive as detection by fluorescent wavelength and do not require any special equipment for visualizing. The gels incubated with the compounds can be inspected as one would with a Coomassie brilliant blue stained gel to determine the presence of an affinity tag containing fusion protein. (See, Example 19)

A detailed description of the invention having been provided above, the following examples are given for the purpose of illustrating the invention and shall not be construed as being a limitation on the scope of the invention or claims.

### EXAMPLES

### Example 1

### Synthesis of compound 1 [7-amino-3-(1-carboxy-1-(bis(carboxymethyl)amino)-5-(acetylamino))pentyl-4-methylcoumarin-6-sulfonic acid, tetratriethylammonium salt]

To a solution of 7-amino-3-((((succinimidyl)oxy)carbonyl)methyl)-4-methylcoumarin-6-sulfonic acid (48 mg, 0.11 mmol) in DMF (3 mL) is added a solution of NTA (34 mg, 0.13 mmol) and triethylamine (0.1 mL) in water (1 mL). The mixture is stirred at room temperature for 15 minutes and then concentrated to dryness in vacuo. The crude product is purified on SEPHADEX LH-20, eluting with water to give pure Compound 1 (59.3 mg).

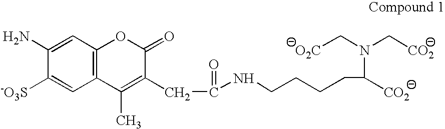

### Example 2

### Synthesis of Compound 2 [4,4-difluoro-4-bora-3a,4a-diaza-s-indacene-3,5-bis((6-(propionyl)amino-2-bis(carboxymethyl)amino)hexanoic acid), hexatriethylammonium salt]

To a solution of 4,4-difluoro-4-bora-3a,4a-diaza-s-indacene-3,5-dipropionic acid (86 mg, 0.26 mmol) in DMF (2 mL) at 10° C. is added O-succinimidyl-N,N,N′,N′-tetramethyluronium tetrafluoroborate (170 mg, 0.56 mmol) and triethylamine (0.087 mL). The mixture is stirred at 10° C. for 15 minutes and then followed by the addition of a solution of NTA (160 mg, 0.61 mmol) and triethylamine (0.4 mL) in water (2 mL). The mixture is stirred at 10° C. for another 30 minutes and then concentrated to dryness in vacuo. The residue is purified on SEPHADEX LH-20 to give compound 2 (50 mg).

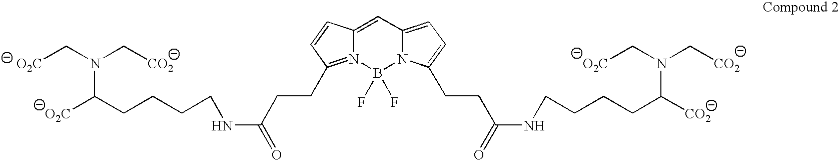

### Example 2A

### Synthesis of Compound 3

Compound 3 is synthesized similar to Compound 2 but with the starting material 4,4-difluoro-1,3,5,7-tetramethyl-4-bora-3a,4a-diaza-s-indacene-2,6-dipropionic acid.

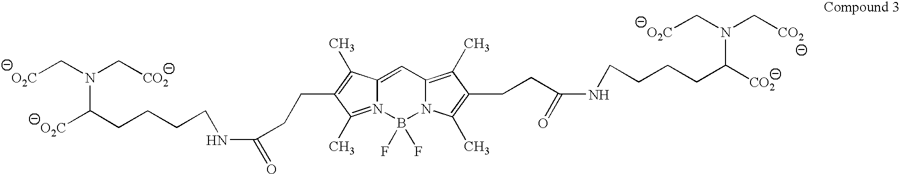

### Example 3

### Synthesis of Compound 4 [7-Hydroxy-6,8-difluoro-3-(1-carboxy-1-(bis(carboxymethyl)amino)-5-(acetylamino))pentyl-4-methylcoumarin, triethylammonium salt]

To a solution of 7-hydroxy-6,8-difluoro-4-methylcoumarin-3-acetic acid, succinimidyl ester (44 mg, 0.12 mmol) in DMF (3 mL) is added a solution of NTA (34.5 mg, 0.13 mmol) and triethylamine (0.1 mL) in water (1 mL). The solution is stirred at room temperature for 30 minutes and then concentrated to dryness in vacuo. The residue is purified on SEPHADEX LH-20 to give compound 4 (40.9 mg).

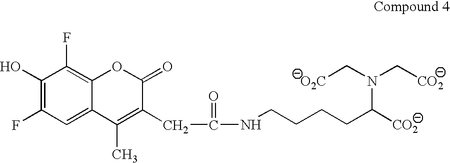

### Example 4

### Synthesis of Compound 5 [7-Hydroxy-3-(1-carboxy-1-(bis(carboxymethyl)amino)-5-(acetylamino))pentyl-4-methylcoumarin]

To a solution of 7-hydroxy-4-methylcoumarin-3-acetic acid, succinimidyl ester (141 mg, 0.427 mmol) in THF (5 mL) is added a solution of NTA (74 mg, 0.282 mmol) and sodium bicarbonate (135 mg, 1.6 mmol) in water (5 mL). The mixture is stirred at room temperature for 15 minutes and then acidified to pH=4 with 0.1 M HCl. The solution is concentrated to dryness in vacuo and the residue is purified on SEPHADEX LH-20, eluting with MeOH:water (1:1) to give compound 5 (55 mg).

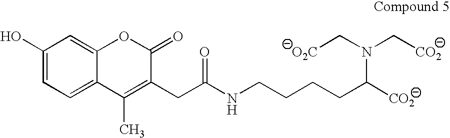

### Example 5

### Synthesis of compound 6 [7-dimethylamino-4-(1-carboxy-1-(bis(carboxymethyl)amino)-5-(acetylamino))pentylcoumarin, trisodium salt]

To a solution of 7-dimethylaminocoumarin-4-acetic acid, succinimidyl ester (100 mg, 0.29 mmol) (1.5 mL) is added a solution of NTA (38 mg, 0.145 mmol) and sodium bicarbonate (61 mg, 0.725 mmol) in water (1.5 mL). The mixture is stirred at room temperature for 15 minutes and then concentrated to dryness in vacuo. The residue is purified on SEPHADEX LH-20, eluting with methanol:water (1:1) to give compound 6.

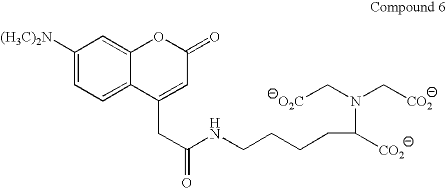

### Example 6

### Synthesis of Compound 7

To a solution of 4,4-difluoro-5,7-dimethyl-4-bora-3a,4a-diaza-s-indacene-3-propionyl ethylenediamine, hydrochloride (BODIPY® FL EDA, Molecular Probes 2390, 20 mg, 0.054 mmol) in 3 mL dry DMF under argon is added DIEA (9 μL, 0.054 mmol), followed by solid DTPA anhydride (Aldrich, 77 mg, 0.22 mmol). The resulting orange mixture is stirred at room temperature for 2 hours and then diluted with 5 mL water. The pH is raised to 9.0 with aqueous KOH. After another 2 hours, the reaction solution is concentrated in vacuo and the product purified by column chromatography on Sephadex LH-20 using E-pure water as eluant to give compound 7 as 22 mg of orange powder.

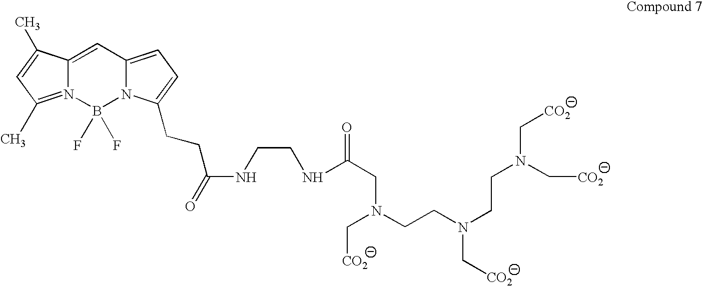

### Example 7

### Synthesis of Compound 8

4,4-difluoro-5,7-dimethyl-4-bora-3a,4a-diaza-s-indacene-3-propionyl ethylenediamine, hydrochloride (BODIPY® FL EDA, Molecular Probes 2390, 7 mg, 0.019 mmol) is dissolved into a mixture of (s)-1-p-isothiocyanatobenzyldiethylenetriaminepentaacetic acid (DTPA isothiocyanate, Molecular Probes 24221, 10 mg, 0.019 mmol) in 2 mL water. The pH (˜3) is raised to 10 with aqueous sodium carbonate. The resulting orange solution is stirred at room temperature for 3.5 hours, then concentrated in vacuo. The residue is purified by column chromatography on Sephadex LH-20 using E-pure water as eluant to give compound 8 as 29 mg of orange powder.

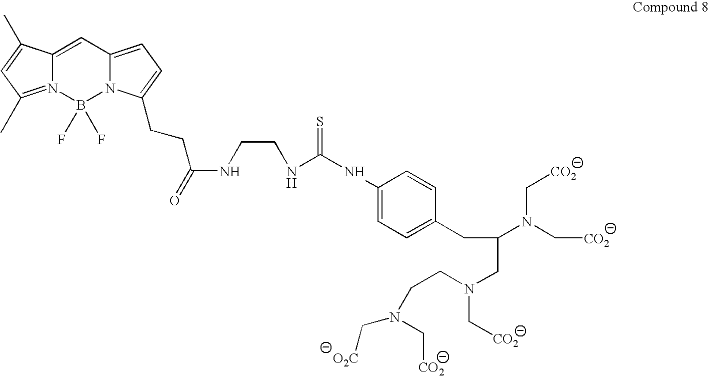

### Example 8

### Synthesis of Compound 9

For the synthesis of carbamate 9a a solution of penta-t-butyl 1-(S)-(p-aminobenzyl)-diethylenetriamine-pentaacetate (prepared according to the published procedure of Donald T. Corson & Claude F. Meares. Bioconjugate Chem., 11(2), 2000, 292-299, 0.800 g, 1.03 mmol) in 20 mL of methylene chloride is added 1 mL of pyridine followed by the addition of a solution of the acid chloride of N—CBZ-6-aminohexanoic acid (0.290 g, 1.02 mmol) in 5 mL of methylene chloride. The reaction mixture is stirred overnight at room temperature and concentrated in vacuo. The residue is dissolved in 100 mL of ethyl acetate and the resulting solution is washed with 10% HCl (2×30 mL), water (30 mL), brine (30 mL) and dried over sodium sulfate. The solution is concentrated and put on a silica gel column (packed with ethyl acetate). The column is eluted first with ethyl acetate to remove impurities and then the desired product is eluted with 10:1 chloroform-methanol. Pure fractions are combined and the solvent evaporated to give amide 9a (0.54 g, 54%) as a viscous oil.

For the synthesis of aminoacid 9b, the carbamate 9a (0.700 g, 0.683 mmol) is dissolved in 10 mL of TFA. The reaction mixture is kept for 3 days at room temperature. Volatiles are evaporated in vacuo and the residue is re-evaporated twice from toluene, leaving a viscous oil. The oil is stirred with ethyl acetate until it solidifies. The resulting solid is filtered and dried in vacuum to give the aminoacid 9b (0.400 g, 96%).

For the synthesis of compound 9, the aminoacid 9b (0.090 g, 0.147 mmol) is suspended in 10 mL of water. The pH is adjusted to pH ˜8 using 1M KOH. The resulting solution is added to a solution of 6-((4,4-difluoro-1,3-dimethyl-5-(4-methoxyphenyl)-4-bora-3a,4a-diaza-s-indacene-2-propionyl)amino)hexanoic acid, succinimidyl ester (BODIPY® TMR-X, SE, MPI 6117, 0.03 g, 0.049 mmol) in 5 mL of DMF. The reaction mixture is stirred overnight at room temperature. The pH is monitored and adjusted to pH ˜8 during the first 2 hrs. The volatiles are removed in vacuo. The residue is re-dissolved in water and put onto a Sephadex LH-20 column. The column is eluted with E-pure water. Pure fractions containing the most polar fluorescent product are combined. The resulting solution is concentrated to ˜3 mL in vacuo and then lyophilized to give Compound 9 as a red powder (0.061 g).

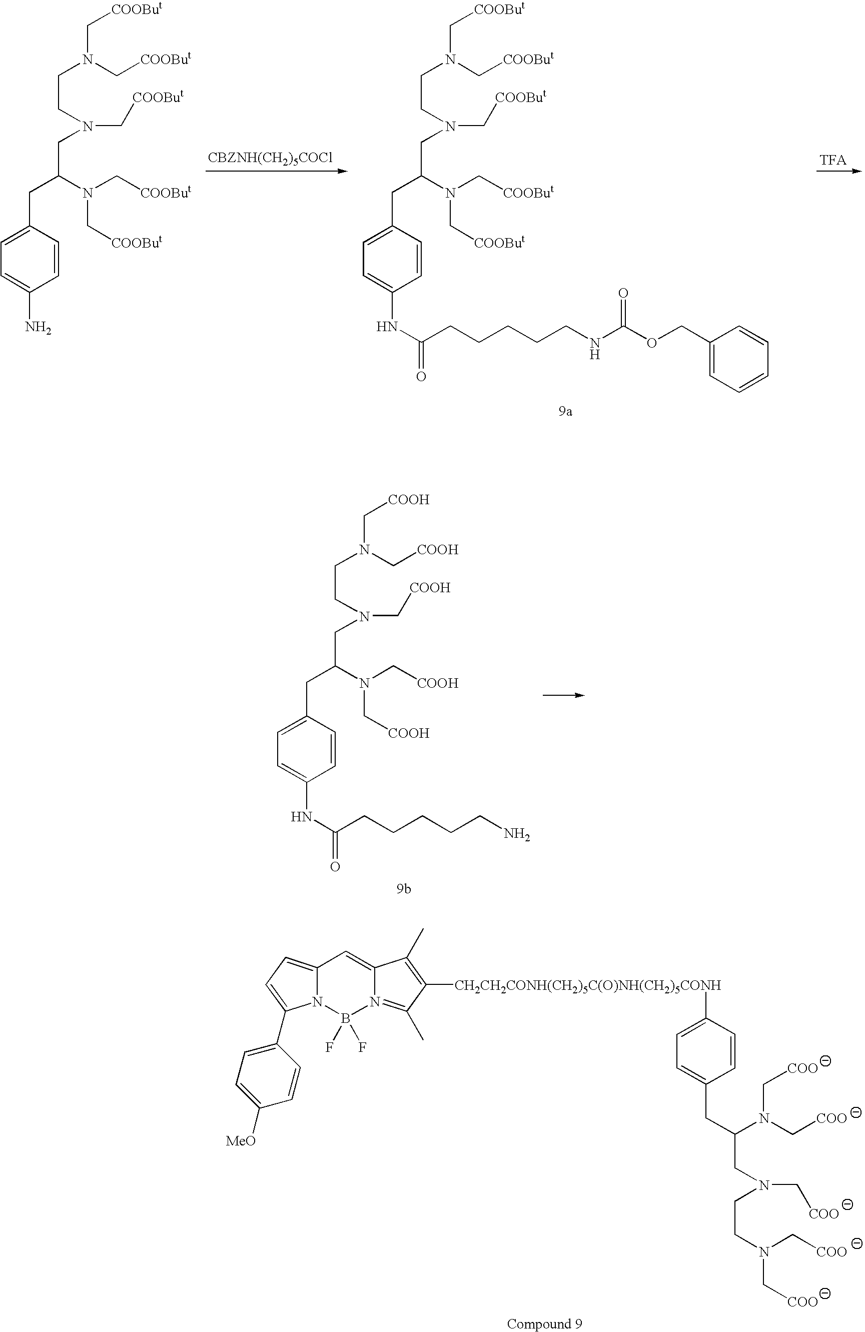

### Example 9

### Synthesis of Compound 10

5-(((4-(4,4-difluoro-5-(2-thienyl)-4-bora-3a,4a-diaza-s-indacene-3-yl)phenoxy)acetyl)amino) pentylamine, hydrochloride (BODIPY® TR cadaverine, Molecular Probes 6251, 10 mg, 0.019 mmol) is dissolved into a mixture of (S)-1-β isothiocyanatobenzyldiethylenetriaminepentaacetic acid (DTPA isothiocyanate, Molecular Probes 24221, 10 mg, 0.019 mmol) in 2 mL water. The pH (˜2) is raised to 10 with aqueous sodium carbonate. The resulting blue solution is stirred at room temperature for two days, then concentrated in vacuo. The residue is purified by column chromatography on Sephadex LH-20 using E-pure water as eluant to give compound 10 as 2 mg of purple powder.

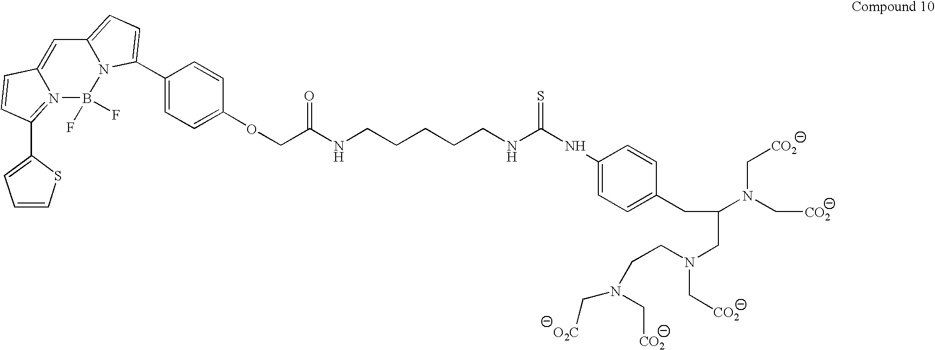

### Example 10

### Synthesis of BODIPY FL-TTHA Compound 11

To a solution of 4,4-difluoro-5,7-dimethyl-4-bora-3a,4a-diaza-s-indacene-3-propionyl ethylenediamine, hydrochloride (BODIPY®) FL EDA, Molecular Probes 2390, 20 mg, 0.054 mmol) in 3 mL dry DMF under argon is added DIEA (9 μL, 0.054 mmol), followed by solid TTHA anhydride (prepared according to Achour et al., Inorganic Chemistry 1998, 37: 2729-2740,100 mg, 0.22 mmol). The resulting orange mixture is stirred at room temperature for 2 hours, then diluted with 5 mL water. The pH is raised to 9.0 with aqueous KOH. After another 2 hours, the reaction solution is concentrated in vacuo and the product purified by column chromatography on Sephadex LH-20 using E-pure water as eluant to give compound 11 as an orange powder.

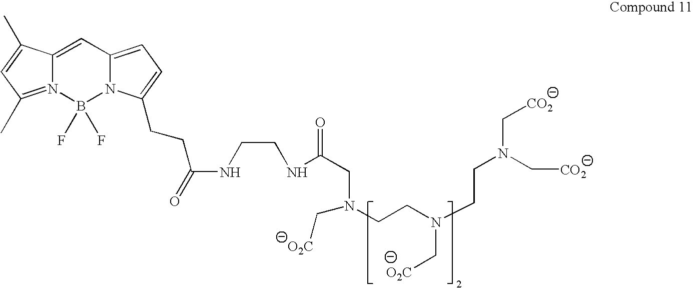

### Example 11

### Synthesis of Compound 13

Nα,Nα-Bis(carboxymethyl)lysine (0.157 g, 0.600 mmol) was dissolved in a mixture of 4.8 mL 1M Et₃NH₂CO₃ buffer and 15 mL water. 4,4-Difluoro-5-(2-pyrrolyl)-4-bora-3a,4a-diaza-s-indacene-3-propionic acid, succinimidyl ester (BODIPY® 576/589 SE, 0.170 g, 0.401 mmol) was dissolved in 30 mL of dioxane and added to the amino acid solution. The reaction mixture was stirred for 1 h at RT and evaporated to dryness. The residue was re-evaporated from water to remove tetraethylammonium salts. The crude product was dissolved in water and loaded onto an LH-20 column (packed in water). The column was eluted with water. Fractions containing pure material were combined and lyophilized to give compound 13 as a dark red powder (0.120 g, 34%) as its triethylammonium salt.

### Example 12

### Synthesis of Compound 14

The triethylammonium salt 13 (0.120 g, 0.137 mmol) was suspended in 5 mL of DMF. i-Pr₂NEt (0.14 mL, 0.82 mmol) was added to the suspension followed by BrCH₂OAc (0.08 mL, 0.8 mmol). The reaction mixture was stirred for 4 hrs at RT and then diluted with brine (30 mL). The product was extracted with ethyl acetate (3×30 mL). The combined extracts were washed with water (3×30 mL), brine (30 mL), dried over anhydrous sodium sulfate and evaporated. The crude product was dissolved in chloroform and loaded onto a silica gel column packed with 4:8:0.1 chloroform-ethyl acetate-acetic acid. The same solvent mixture was used to elute the column. Fractions containing pure product were combined and evaporated in vacuo. The residue was re-evaporated from toluene to give AM ester 14 as a dark purple wax (0.081 g, 75%).

### Example 13

### Synthesis of Compound 15

p-Nitrophenylalanine methyl ester hydrochloride 15a (Bachem, cat. # F-1910; 2.00 g, 7.68 mmol) was added portionwise to 9.9 mL (92 mmol) of diethylenetriamine with stirring at RT. When all hydrochloride was added the mixture was stirred for additional 5 hrs at RT. Excess of diethylenetriamine was removed in vacuum. The residue was dissolved in 20 mL of conc. ammonia solution and the product was extracted with CH₂Cl₂ (10×20 mL). The combined extracts were dried over sodium sulfate and concentrated in vacuum to give amide 15b (1.98 g, 87%) as yellow oil.

Amide 15b (1.98 g, 6.71 mmol) was dissolved in 60 mL of dry THF. BH₃.THF complex (1M solution of in THF, 60.4 mL, 60.4 mmol) was added to amide 15b dropwise under nitrogen with stirring and cooling (ice/water bath). After all amount of complex was added, the temperature was allowed to rise to ambient and the mixture was stirred under reflux for 15 hrs. Then the mixture was cooled again (ice/water bath) and excess of BH₃ was carefully decomposed with water (5 mL, dropwise, stirring). The resulting solution was concentrated in vacuum and the residue mixed with 35 mL of water and 35 mL of conc. HCl. The solution was stirred for 3.5 hrs under reflux then 20 hrs at RT and evaporated to dryness. The residue was mixed with 50 mL of conc. ammonia and 50 mL of water. The product was extracted with chloroform (6×100 mL). The combined extracts were dried over Na₂SO₄ and evaporated to give amine 15c (1.37 g, 73%) as yellow oil.

Amine 15c (1.37 g, 4.88 mmol) was dissolved in 50 mL of DMF. Diisopropyethylamine (12.7 mL, 72.9 mmol) and tert-butyl bromoacetate (8.64 mL, 58.5 mmol) were added to the solution, followed by addition of powdered KI (0.89 g, 5.4 mmol). The reaction mixture was stirred for 72 hrs at RT and evaporated to dryness. The residue was mixed with 100 mL of water and the product extracted with diethyl ether (3×40 mL). The combined extracts were washed with water (40 mL), brine (40 mL), dried over sodium sulfate and evaporated. The crude product was dissolved in 2:1 hexanes—ethyl acetate mixture and loaded on silica gel column (packed with 2:1 hexanes—ethyl acetate mixture). The same solvent mixture was used to elute the column. Pure fractions were combined and evaporated to give hexaester 15d as yellow oil (1.68 g, 36%).

Ester 15d (1.68 g, 1.74 mmol) was dissolved in 50 mL of methylene chloride. 10% Pd/C (100 mg) was added to the solution and the mixture was shaken in Parr Apparatus at 50 psi for 4 hrs. The catalyst was filtered off, and the solution vas concentrated in vacuum. The residue was dissolved in 9:1 CH₃CN:water mixture and the solution was loaded on silica gel column (packed with 9:1 CH₃CN:water mixture). The column was eluted with the same solvent mixture. Pure fractions were combined and concentrated in vacuum to give amine 15e as yellow oil (1.54 g, 95%).

N—CBZ-6-aminohexanoic acid 15f (0.600 g, 2.26 mmol) was dissolved in 5 mL of methylene chloride. DCC (0.234 g, 1.13 mmol) was added to the solution and reaction mixture was stirred over weekend at RT. The precipitate was filtered off and washed with 2 mL of methylene chloride. Methylene chloride solutions were combined and evaporated to give anhydride 15g (0.58 g, quant.).

Amine 15e (0.600 g, 0.640 mmol) was dissolved in 5 mL of DMF. i-Pr₂NEt (0.54 mL, 3.1 mmol) was added to the solution followed by addition of anhydride 15g (0.58 g, 1.13 mmol) as a solution in 3 mL of DMF. The reaction mixture was stirred overnight at RT. The solution was diluted with 80 mL of 0.5M KOH and the product was extracted with EtOAc (3×40 mL). The combined extracts were washed with water (3×30 mL), brine (30 mL), dried over sodium sulfate and evaporated. The crude product was suspended in 1:1 hexanes—EtOAc mixture and loaded on silica gel column (packed with 2:1 EtOAc—hexanes mixture. The column was eluted first with 2:1 EtOAc—hexanes mixture and then with 10% MeOH in chloroform. Fractions containing pure amide were combined and evaporated to give desired amide 15h as viscous oil (0.846 g).

CBZ protected amide 15h (0.84 g, 0.71 mmol) was dissolved in 5 mL of TFA. The solution was kept at RT for 72 hrs, and then evaporated in vacuum. The residue was re-evaporated from toluene (3×20 mL) and triturated with EtOAc. White precipitate formed. Mixture was centrifuged, supernatant separated and solid washed with fresh EtOAc. Mixture was stirred and centrifuged again. After supernatant was removed the procedure was repeated three more times and then the product was dried in vacuum to give amine 15i as a white solid (0.521 g, 89%).

Amine 15i (0.054 g, 0.065 mmol) was dissolved in 4 mL of DMF. i-Pr₂NEt (0.023 mL, 0.13 mmol) was added to the solution. White precipitate formed. Water was added to the solution (about 2 mL) until all solid dissolved. SE ester D6117 (0.02 g, 0.033 mmol) was dissolved in 2 mL of DMF and two solutions were mixed. After stirring for 20 min. 0.5 g of sodium bicarbonate was added to the solution and the reaction mixture was stirred for 48 hrs at RT. Reaction mixture was concentrated in vacuum, the residue dissolved in water (4 mL) and loaded on LH-20 column. The column was eluted with water. Fractions containing most polar fluorescent product were combined and concentrated to the volume ˜10 mL. Solution was acidified with 1 mL of 10% HCl and the product was extracted with n-BuOH (3×10 mL). The combined extracts were concentrated in vacuum and the residue was mixed with 10 mL of water. The solid was filtered off, washed with water (2 mL) and dissolved in 5% ammonia (˜2 mL). The resulting solution was loaded on LH-20 column ant eluted with water. Pure fractions were combined concentrated in vacuum and lyophilized to give amide 15 (0.014 g of Compound 15, isomer a and 0.022 g of Compound 15, isomer b. Both fraction show similar purity by LCMS). MS+H: 1257 (calculated for C₅₈H₇₈N₉O₁₆BF₂.3NH₃: 1256).

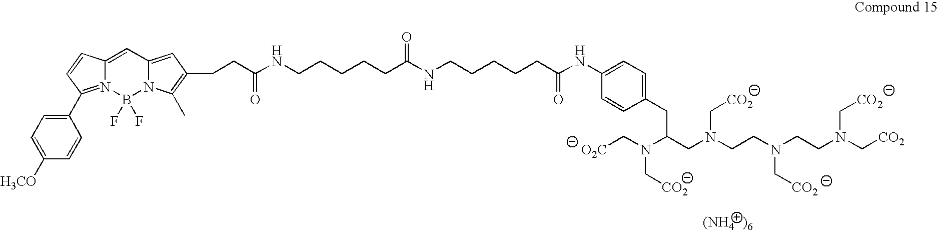

### Example 14

### Synthesis of Compound 16

NTA (0.100 g, 0.382 mmol) was dissolved in 2 mL of water. The pH of the solution was adjusted to 8 using 1M KOH. (D6117, Molecular Probes, Inc.) (0.100 g, 0.164 mmol) was dissolved in 2 mL of DMF and added to solution of amino acid. The reaction mixture was stirred for 2 hrs at RT. During the reaction pH was monitored and adjusted to 8 with 1M KOH. After all SE ester (D6117) was consumed the reaction mixture was evaporated. The residue was dissolved in water and solution was loaded on LH-20 column. The column was eluted with water. Fractions containing pure product (TLC, A/B 1:1) were combined, concentrated to the volume of 2-3 mL, and lyophilized to give Compound 16 (0.130 g, 91%). [MS-H] 754.3, calculated for C₃₇H₄₈N₅O₉BF₂755.6. Solution A: dioaxane:i-PrOH:water:ammonia 80:40:68:72. Solution B: dioaxane:i-PrOH:water:ammonia 15:58:13:14

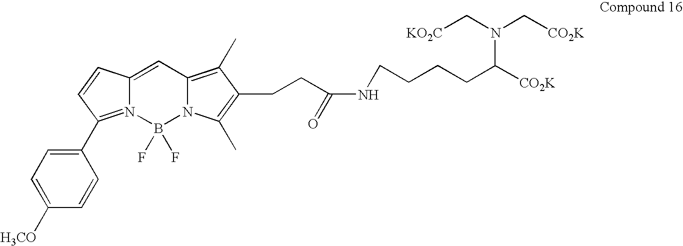

### Example 15

### Detection of Fusion Proteins Containing a Poly-Histidine Affinity Tag in Polyacrylamide Gels

Eschericia coli BL21 DE3 cells were transformed with plasmids containing either the human ATP synthase α subunit, the d subunit (including the leader sequence) or urate oxidase. Both proteins were constructed to have a poly-histidine affinity tag comprising six histidine residues, at the N-terminus and could be induced by isopropyl-beta-D-thiogalactoside (IPTG) addition to the medium. Pre-cultures (10 ml) were grown overnight in bacterial cell culture medium (LB medium) at 37° C. with constant shaking. The next day 100 μl was transferred to 50 ml of fresh LB medium containing 0.1 mg/ml ampicillin and grown until they reached an optical density at 595 nm (OD₅₉₅) of 0.8. At this point 5 ml of culture was removed and immediately frozen on dry ice. To the rest of the culture 0.8 mM IPTG was added to induce the over-expression of the subunits. Samples (5 ml each) were taken after 10 min, 30 min, 1 h, 1.5 h, 2 h, 2.5 h, and 3 h and again frozen on dry ice.

The cells from the different time points were pelleted (at 5000×g) and the supernatant was discarded. The cells were lysed adding 200 μl of buffer 1 (0.3% SDS, 200 mM DTT, 28 mM Tris base, 28 mM Tris HCl, pH 8.0) and incubated for 10 min, followed by a short (2 min) sonication to break the cells open completely. To remove the DNA, 20 μl of buffer II (24 mM Tris Base, 476 mM Tris HCl, 50 mM MgCl₂, 1 mg/ml DNAse I, 0.25 mg/ml RNAse A) was added and the cell extract was incubated for another 10 min. Finally, 100 μl of the cell extract was removed and mixed with 40 μL of 5×SDS sample buffer (290 mM Tris, 25% glycerol, 250 mM DTT, 10% SDS, 0.01% bromophenol blue). After vortex mixing, the samples were centrifuged at maximum speed (˜12,000×g) in a microcentrifuge and the supernatant was subjected to SDS-polyacrylamide gel electrophoresis.

Proteins were separated by SDS-polyacrylamide gel electrophoresis utilizing 13% T, 2.6% C gels. % T is the total monomer concentration expressed in grams per 100 ml and % C is the percentage crosslinker. The 0.75 mm thick, 6×10 cm gels were subjected to electrophoresis using the Bio-Rad mini-Protean III system according to standard procedures.

Following separation of the proteins on a SDS-polyacrylamide gel, the gels were fixed for 20 minutes in 100 ml of 50% ethanol/7% acetic acid and then fixed overnight in 100 ml of fresh fixative solution to ensure complete elimination of SDS. Gels were next washed 3 times for 20 minutes each in deionized water. The gels were then incubated in a staining solution containing 10 μM compound 1 or 2 μM compound 2; 100 μM NiCl₂; 50 mM PIPES at pH 6.5 for 45-90 minutes in a total volume of 25 ml. Afterwards, the gels were washed 2 to 4 times for 20 minutes each in deionized water. In order to ensure that the optimal signal was documented, gels were imaged after the second and fourth wash.

The resulting blue-fluorescent signal produced by compound 1 was visualized using 300 nm trans-illumination and 520 nm band pass emission filter on the Lumi-Imager (Roche Biochemicals, Indianapolis, Ind.), a cooled CCD-camera based system digitizing at 1024×1024 pixels resolution with 16-bit gray scale levels assigned per pixel. Alternatively, the signal was visualized utilizing a UVP transilluminator/Polaroid MP4+ camera system (UVP, Upland, Calif.) with 365 nm transillumination and photographed with Polaroid 667 black-and-white print film using a SYPRO® protein gel stain photographic filter (Molecular Probes, Eugene, Oreg.).

The resulting green-fluorescent signal produced by compound 2 was visualized using the 473 nm excitation line of the SHG laser on the Fuji FLA-3000G Fluorescence Image Analyzer (Fuji Photo, Tokyo, Japan) with the 520 nm long pass filter or the 580 nm band pass filter, respectively. See, FIGS. 1 and 2.

### Example 16

### Detection of Fusion Proteins Containing a Poly-Histidine Affinity Tag in Polyacrylamide Gels that are First Separated by Isolelectric Focusing

E. coli cultures of induced and un-induced human ATP synthase d subunit (100 ml each) were grown as described in Example 11 and the cells were pelleted at 5000×g. The cells were resuspended in 2 ml of 25 mM Tris, pH 7.5 before addition of 4 ml of 28 mM Tris base, 22 mM Tris HCl, 0.3% SDS to lyse the cells. After 5 minutes, a sufficient amount of 1 M MgCl₂ was added to make a final concentration of 5 mM, followed by 10 μl RNAse A (10 mg/ml) and 40 μl DNAse 1 (10 mg/ml) to digest the nucleic acids. The raw cell extract was then mixed with 6 ml Urea buffer (7 M Urea, 2 M Thiourea, 2% Chaps, 1% Zwittergent 3-10, 65 mM DTT) and insoluble material was pelleted by centrifigation (15,000×g, SS34 rotor). The supernatant was then injected into the Rotofor chamber (Bio-Rad Laboratories, Hercules, Calif.) according to the manufacturers manual using the same urea buffer in the chamber. The proteins were focused for roughly 3 h before harvesting into 20 fractions spanning a pH range of 2-12. Fractions were collected using the system's vacuum manifold and were acetone-precipitated and resuspended in SDS-sample buffer. For SDS polyacrylamide gel electrophoresis 30 μl of sample per fraction was utilized and gels were subsequently stained for the presence of the oligopoly-histidine affinity tag using Compound 2 as described in Example 11.

### Example 17

### Serial Dichromatic Detection of Poly-Histidine Affinity Tag and Total Protein in SDS-Polyacrylamide Gels

Following selective staining of the poly-histidine affinity tag containing fusion proteins separated on a SDS-polyacrylamide gel, as described in Example 15, the gel was incubated overnight with SYPRO® Ruby protein gel stain with gentle orbital shaking, typically 50 rpm. The gel was then incubated in 7% acetic acid, 10% methanol for 30 minutes, also at 50 rpm. The fluorescent signal from the affinity tag containing proteins and non-affinity tag proteins was collected with a standard CCD camera-based imaging system with 300 nm UV light excitation and a 600 nm bandpass filter.

### Example 18

### Detection of Poly-Histidine Affinity Tag Containing Fusion Proteins in Two-Dimensional Polyacrylamide Gels

E. coli BL21 DE 3 cells expressing poly-histidine affinity tag ATP synthase d subunit induced with IPTG were prepared and a lystate (100 μl) was diluted in urea buffer (2 M Thiourea, 7 M Urea, 2% CHAPS, 1% Zwittergent 3-10, 0.8% Ampholytes 3-10, 56 mM DTT) and applied on a first dimension IPG strip (3-10 non linear, 18 cm; Amersham Pharmacia) that had been rehydrated overnight in urea buffer. The strips were overlayed with 2 ml of light mineral oil and the proteins focused for 24.5 h, at 70 kVh and 20° C. for a final voltage of 100 μA/strip. The IPG strips were equilibrated in 300 mM Tris/Base, 75 mM Tris/HCl, 3% SDS, 50 mM DTT, 0.01% Bromophenol Blue for 10 min and then laid on top of a 12.5% SDS-polyacrylamide gel. Electrophoresis was performed according to standard procedures for 4.5 h.

After the second dimension electrophoresis the gels were fixed in 10% ethanol, 7% acetic acid overnight to remove SDS. The next day the gels were washed twice with dH₂O for 20 minutes each before equilibration in 50 mM PIPES, 1 mM NiCl₂, pH 6.5. The gels were washed again twice for 15 minutes each before staining with 10 μM Compound 1 in 50 mM PIPES, pH 6.5 (250 ml). To remove excess dye the gels were washed twice in dH2O for 20 minutes each. The staining was imaged on a Lumi-Imager (Roche) using UV light excitation and a 520 nm emission filter with a 5 s exposure time.

Following detection of poly-histidine affinity tag containing fusion proteins, the gels was stained for total protein using SYPRO® Ruby protein gels stain as described in Example 17.

### Example 19

### Detection of Fusion Proteins Containing a Poly-Histidine Affinity Tag in Polyacrylamide Gels using a Colorimetric Fluorescent Compound

Fusion proteins containing a poly-histidine affinity tag were prepared and separated from Eschericia coli lysate proteins by SDS-polyacrylamide gel electrophoresis as described in Example 15. Following separation of the proteins on a SDS-polyacrylamide gel, the gels were fixed for 20 minutes in 100 ml of 50% ethanol/7% acetic acid and then fixed overnight in 100 ml of fresh fixative solution to ensure complete elimination of SDS. Gels were next washed 3 times for 20 minutes each in deionized water. The gels were then incubated in a staining solution containing 10 μM compound 9 or compound 10; 10 μM NiCl₂; 50 mM PIPES at pH 6.5 for 45-90 minutes in a total volume of 25 ml. Afterwards, the gels were washed 2 to 4 times for 20 minutes each in deionized water. The colorimetric signal from the poly-histidine affinity tag containing proteins was detected with a standard CCD camera-based imaging system with white light illumination and no filter according to standard Coomassie Blue or silver staining imaging methods.

### Example 20

### Detection of Glutathione S-Transferase (GST) with Texas Redo X-Glutathione compound in Polyacrylamide Gels

A purified sample of GST was separated by SDS-polyacrylamide gel electrophoresis utilizing 13% T, 2.6% C gels. % T is the total monomer concentration expressed in grams per 100 ml and % C is the percentage crosslinker. The 0.75 mm thick, 6×10 cm gels were subjected to electrophoresis using the Bio-Rad mini-Protean III system according to standard procedures.

Following separation of the protein on a SDS-polyacrylamide gel, the gel was fixed for 1 hour in 100 ml of 50% methanol/10% acetic acid and then fixed overnight in 100 ml of fresh fixative solution to ensure complete elimination of SDS. Gels were next washed 3 times for 20 minutes in deionized water. The gels were then incubated in a staining solution containing 5 μM Texas Red X-glutathione compound in 50 mM PIPES at pH 6.5 for 90 minutes in a total volume of 50 ml. Afterwards, the gels were washed 2 times for 20 minutes each in deionized water.

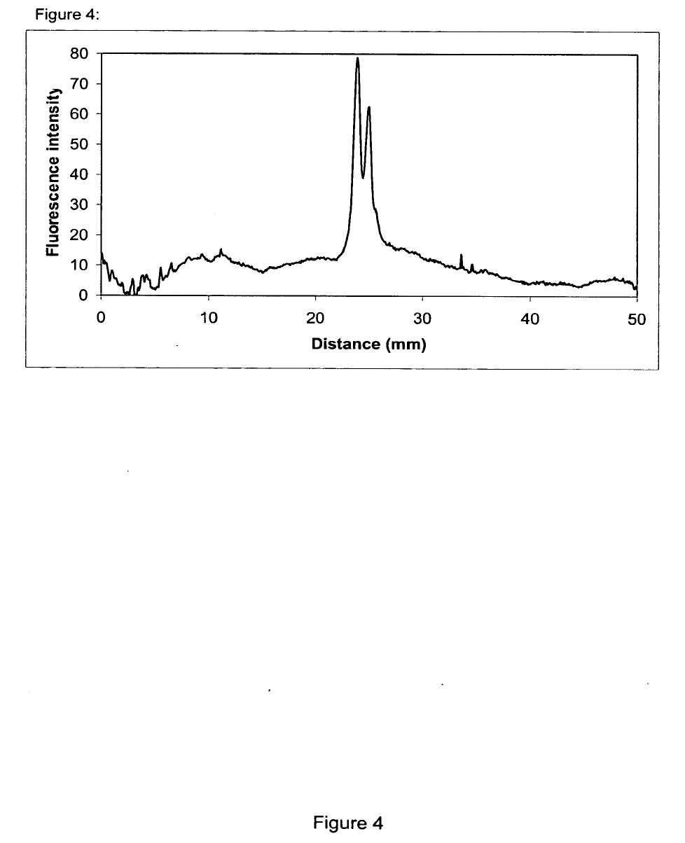

The resulting red-fluorescent signal produced by Texas Red-glutathione was visualized using the 532 nm excitation line of the SHG laser on the Fuji FLA-3000G Fluorescence Image Analyzer (Fuji Photo, Tokyo, Japan) and 580 band pass emission filter. See, FIG. 4.

### Example 21

### Detection of Fusion Proteins Containing a Poly-Histidine Affinity Tag on a Membrane Blot

Escherichia coli lysates containing 6× histidine-tagged A subunit of ATPase and 6× histidine-tagged porin are fractionated by 13% T, 0.8% C SDS-polyacrylamide gel electrophoresis and electroblotted onto PVDF membrane. Blots are wetted with 100% methanol and then fixed with 50% methanol/7% acetic acid, briefly rinsed in deionized water and then stained for 15 minutes with either Pro-Q Sapphire 488 or Pro-Q Sapphire 532 gel stain solution. Blots are destained with two five-minute washes in 50 mM PIPES, pH 6.5, 20% acetonitrile to obtain fairly specific detection of the two his-tagged proteins. Blots are briefly washed in water and then dried before imaging. With both dyes, the two oligohistidine-tagged proteins are readily distinguished from other proteins in the lysate as brightly fluorescing bands. Limits of detection are approximately 20 ng.

### Example 22

### Detection of Fusion Proteins Containing a Poly-Histidine Affinity Tag on a Microarray

Purified oligohistidine-tagged fusion proteins (the a subunit of Escherichia coli ATPase and porin), as well as control proteins (bovine serum albumin and ovalbumin) are arrayed from a source plate (384 well plate) concentration of 0.468 μg/ml-0.240 mg/ml in water, onto HydroGel coated slides (Perkin Elmer), using the BioChip Arrayer™ (Perkin Elmer). The BioChip Arrayer™ utilizes a PiezoTip™ Dispenser consisting of 4 glass capillaries. Proteins are dispensed from the PiezoTip™ by droplets 333 μl in volume to create array spots ˜200 microns in diameter with a 500 micron horizontal and vertical pitch (pitch=center to center spacing of spots). Proteins are arrayed in duplicate in four rows, with 10 dilution points. The resulting concentration range of the array is 166.5 μg/spot-0.325 μg/spot. For detection of oligohistine-tagged proteins, slides are incubated for 45 minutes on a rotator in 50% ethanol/7% acetic acid and then fixed overnight in fresh fixative solution to ensure complete elimination of SDS. Microarrays are next washed 3 times for 20 minutes each in deionized water. The microarrays are then incubated in a staining solution containing 10 μM Compound 2 or Compound 15; 50 mM PIPES at pH 6.5 for 45-90 minutes. Afterwards, the microarrays are washed 2 to 4 times for 20 minutes each in deionized water. In order to ensure that the optimal signal was documented, gels are imaged after the second and fourth wash. Slides are then spun briefly in a microarray high-speed centrifuge affixed with a rotor with a slide holder (Telechem) at ˜6000 rpm to remove excess liquid. After slides are dry, the arrays are imaged using the ScanArray® 5000 XL Microarray Analysis System (Packard Instrument Co., Meriden, Conn.) using the 488 nm laser and 522 nm emission filter. The oligohistine tagged proteins are detected as discrete fluorescent spots, while little or no signal generated on the control proteins. Detection sensitivity is less than 20 pg.

### Example 23

### Detection of Fusion Proteins Containing Poly-Arginine Affinity Tag in a Polyacrylamide Gel

An Escherichia coli lysate containing an expressed oligo-arginine-tagged fusion protein (porin) is separated by SDS-polyacrylamide gel electrophoresis utilizing 13% T, 2.6% C gels. % T is the total monomer concentration expressed in grams per 100 ml and % C is the percentage crosslinker. The 0.75 mm thick, 6×10 cm gels are subjected to electrophoresis using the Bio-Rad mini-Protean III system according to standard procedures. Following separation of the proteins on a SDS-polyacrylamide gel, the gels are fixed for 20 minutes in 100 ml of 50% ethanol/7% acetic acid and then fixed overnight in 100 ml of fresh fixative solution to ensure complete elimination of SDS. Gels are next washed 3 times for 20 minutes each in deionized water. The gels are then incubated in a staining solution containing 10 μM compound 1 or 2 μM compound 2; 100 μM NiCl₂; 50 mM PIPES at pH 6.5 for 45-90 minutes in a total volume of 25 ml. Afterwards, the gels are washed 2 to 4 times for 20 minutes each in deionized water. In order to ensure that the optimal signal is documented, gels are imaged after the second and fourth wash.

### Example 24

### Synthesis of Compound 17

128 mg of N_{α},N_{α}-Bis(carboxymethyl)-L-lysine hydrate (Fluka Cat#14580, FW=262.26+H₂O) in a 4 mL vial was neutralized with 1 M sodium carbonate (about 600 μL). An additional 200 μL of sodium carbonate was added. The volume of the solution was increased to 2 mL with ultrapure water. 60 mg of 2-iminothiolane hydrochloride (Aldrich Cat# 33,056-2, FW=137.63) was added to the vial and mixed thoroughly. The resulting solution was mixed for 1 hour at room temperature on a rocker. 5 mg of 4,4-difluoro-3,5-di(iodoacetamidomethyl)-4-bora-3a,4a-diaza-s-indacene (BODIPY® FL bis-(methyleneiodoacetamide)) (Molecular Probes Cat# 10620, FW=585.92 g/mol) was dissolved in 400 μL of dry dimethylformamide and mixed thoroughly to dissolve the dye. The dye solution was transferred dropwise with mixing to the vial containing the derivatized N_{α},N_{α}-(carboxymethyl)-L-lysine. The reaction was allowed to proceed for 2 hours to provide Compound 17. The bis-chelate (Compound 17) was isolated using a C-18 sep-pak cartridge (Waters). The C-18 cartridge was prepared for loading by washing first with 10 mL of 100% acetonitrile, then with 20 mL of ultrapure water, and finally with 10 mL of 0.1 M Tris pH 8.0. The reaction mixture was poured into 8 mL of 0.2 M Tris pH 8.0 and then passed through the sep-pak cartridge. The sep-pak was washed with an additional 10 mL of 0.2 M Tris pH 8.0, and then the column was washed with 5% acetonitrile/water and 10 mL fractions were collected. The fractions were checked for the presence of the dye by absorbance measurements at 488 nm. Fractions containing the dye were combined in a 100 mL flask. The dye was loaded with nickel by addition of 10 mL of 200 mM nickel sulfate and allowed to equilibrate for 30 minutes to prepare Compound 17a. A new C-18 sep-pak cartridge was prepared by washing sequentially with a) 100% acetonitrile, b) ultrapure water and c) 0.1 mM Tris pH 8. The nickel-dye solution was passed through the sep-pak cartridge with the loaded dye binding to the C-18 resin. The sep-pak cartridge was washed with 20 mL of water, and the nickel loaded dye was eluted with 50% acetonitrile in water. The concentration of the eluted dye solution was obtained by measuring the absorbance of a 1:5000 dilution of the dye at 488 nM. 4.2 mL of a 1.1 μM solution of the Bodipy-FL bis-nickel chelate was obtained.

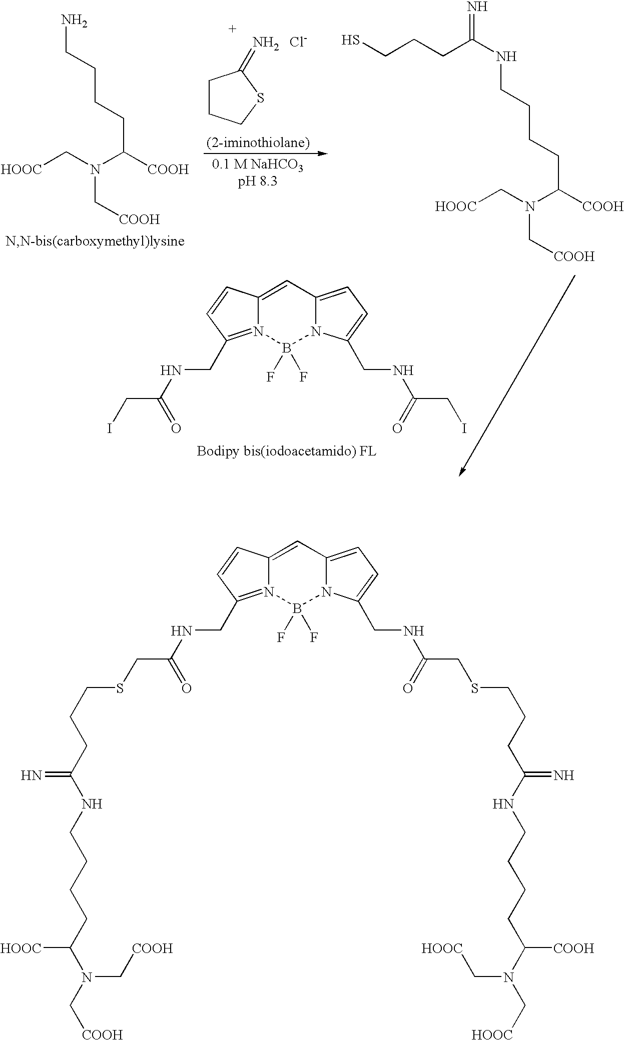

In the structure of Compound 17 described above, the boron difluoro group may be replaced with any structure than can be used to lock the rings together, such as C═O bonded to the nitrogens of the pyrrol rings. In addition, 1,3-imidazole rings may be used in place of the pyrrol rings. There may be one or more non-hydrogen R substituents on the pyrrol rings. The R substituents may be the same or different. The R groups are as defined above. Useful R substituents include alkyl, phenyl rings, phenyl rings fused to one or both of the pyrrol rings, or —CH═CH-Ph, where Ph is a phenyl ring.

In-gel staining using Compound 17a (17 complexed with nickel) was performed after diluting the compound to 0.2 μM in 20 mM phosphate pH 7.8 to prepare the staining solution. A 4-12% NUPAGE™ Bis-Tris gel was run at 200 V constant current for 38 min. The gel was stained as in other Examples. Following staining the gel was imaged in the Fuji-LAS-1000 luminometer and exposed for 2 minutes.
The gel was stained using the following procedure:

TABLE 2
Staining Protocol for NuPAGE ™ 4-12%
Bis-Tris Gel with Compound 17a

| Step | Solution | Time | |
|---|---|---|---|
| Fix | 10% Acetic acid/40% ethanol in water | 1 | hour |
| Wash | Ultrapure water | 10 | min |
| Wash | Ultrapure water | 10 | min |
| Stain | 0.2 M Compound 17a in 20 mM Phosphate pH 7.8 | 1 | hour |
| Wash | 20 mM Phosphate pH 7.8 | 10 | min |
| Wash | 20 mM Phosphate pH 7.8 | 10 | min |

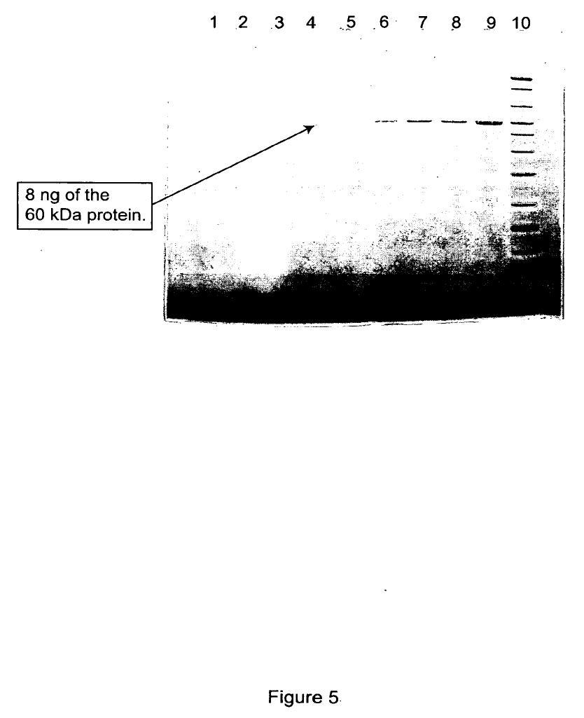

Compound 17a permits in-gel detection of 6xHis tagged proteins, See FIG. 5.

### Example 25

### Synthesis of Compound 18 and 19

16.7 mg of N_{α},N_{α}-Bis(carboxymethyl)-L-lysine hydrate (Molecular Probes; Eugene, Oreg.) was placed in a 2.0 mL microcentrifuge tube. 300 μL of 1M sodium bicarbonate was added. The release of carbon dioxide was observed by formation of gas bubbles. The pH of the solution was adjusted to 9.0±0.2 using 50 μL aliquots of 1N sodium hydroxide.

A mixture of 5 mg Bis-(4-carboxypiperidinyl)sulfone-rhodamine, di(succinimidyl ester) (Molecular Probes; Eugene, Oreg.) and 400 μL of DMF was prepared in a separate vial. The contents were mixed well to dissolve the dye in the DMF solvent. The contents of the microcentrifuge tube was added to the vial, and mixed thoroughly. The vial was placed on a rocker plate for two hours at room temperature.

The reaction mixture was diluted in 10 mL of 0.2 M Tris pH 8.0. A Waters C-18 SEP-PAK cartridge (Waters Corp.; Milford, Mass.; SEP-PAK is a registered trademark of Waters Investments Ltd.; New Castle, Del.) was prepared by washing with 10 mL of 100% acetonitrile at 1-2 mL/minute, then 10 mL of ultrapure water, then 10 mL of 0.2 M Tris pH 8.0. The solution of diluted dye was passed through the cartridge, with the dye binding to the reverse phase resin bed. The resin was washed with 10 mL of 0.2 M Tris pH 8.0 to provide Compound 18. Compound 18 was eluted with either ultrapure water or 5% acetonitrile in water. Fractions containing Compound 18 were identified by their rose color.

Next, Compound 18 was loaded with nickel by combining the fractions, and adding 7 mL of 200 mM nickel sulfate to prepare Compound 19. The mixture was set at room temperature for 15 minutes.

A new Waters C-18 SEP-PAK cartridge was prepared by washing with 10 mL of 100% acetonitrile at 1-2 mL/minute, then two sequential washes with 10 mL of ultrapure water. Compound 19 was loaded onto the cartridge using a plastic syringe. The cartridge was washed twice with 20 mL of ultrapure water. Compound 19 was eluted with 4 mL of a 1:1 mixture of acetonitrile and ultrapure water.

An aliquot of the stock solution (Compound 19) was diluted such that the absorbance at 560 nm is less than 1.0 AU and greater than 0.1 AU. The UV spectrum of the diluted aliquot was determined at 300 nm and 550 nm. The molarity of the stock solution was determined using the following formula. Molarity=(reading at 560 nm×dilution factor)/(extinction coefficient×path length). The extinction coefficient is 120000 L/mol-cm, and the path length is 1 cm for a conventional UV spectrophotometer.

The identity of Compound 19 was further confirmed by obtaining a Maldi-TOF spectrum, which gave the expected peak of 1079 (parent peak minus two nickel ions). The purity of the dye was analyzed using HPLC with an analytical C-18 reverse phase column (4.6 mm×150 mm). A gradient of 0-30% buffer B (90% acetonitrile in water) in buffer A (20 mM Tris pH 8.0) over 20 minutes was used to obtain a purity by integration of over 90%. Formulations of Compound 19 can be prepared in buffers such as 20 mM sodium phosphate pH 8.0. As an example, a 0.2 μM solution was prepared in 20 mM sodium phosphate pH 8.0 to be used as a 1× staining solution.

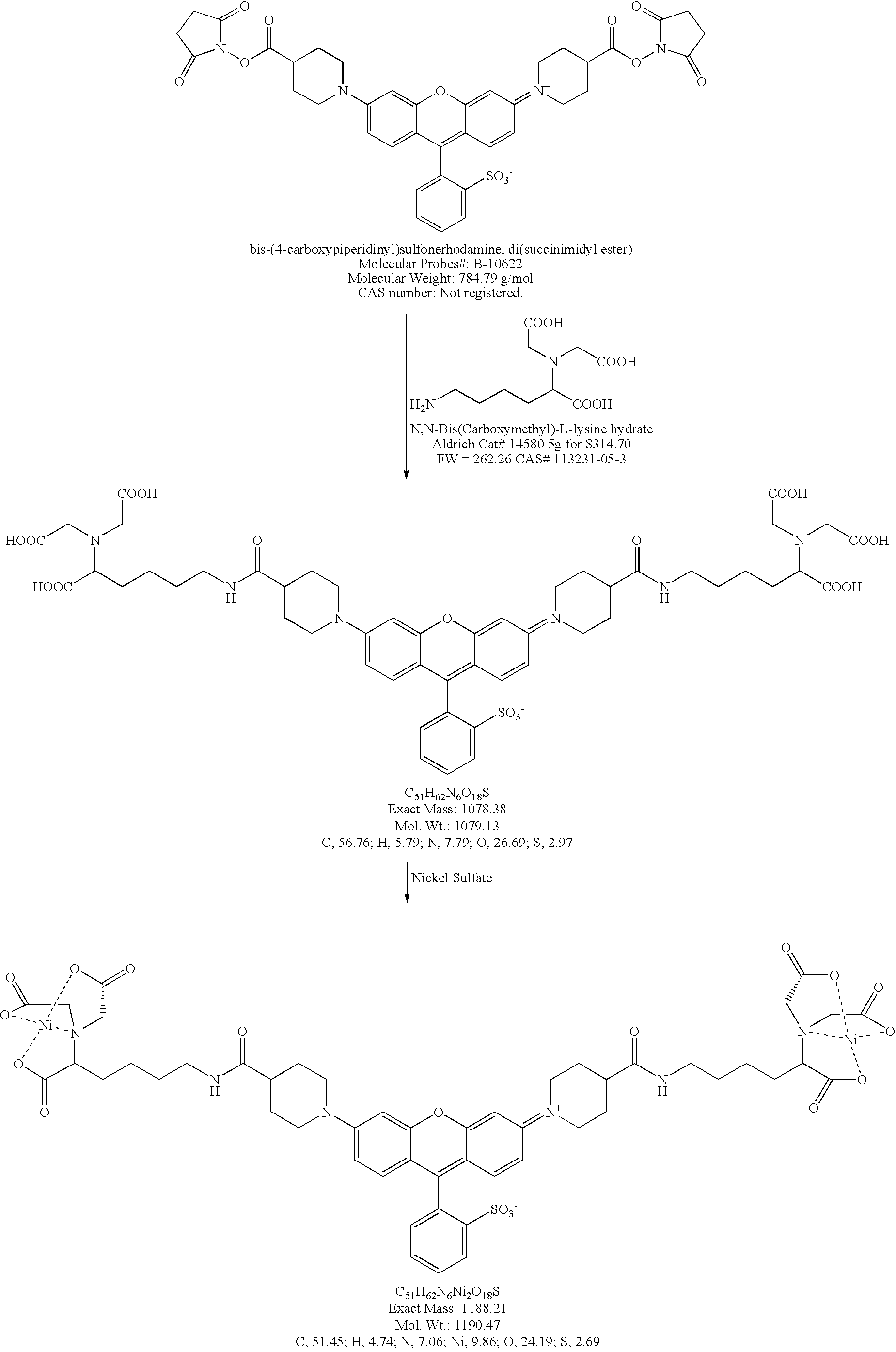

Using Compound 19 at 0.2 uM in 20 mM phosphate pH 7.8, images from the Alpha innotech system using 300 nM transillumination and a 100 nM band pass filter centered at 590 nM was obtained.

### Example 26

### Laser-Based Detection

In addition to using a standard transilluminator and video camera, Compound 18 and 19 can be detected using a laser based scanner. A dilution series of the 60 kDa BenchMark™ proteins were run on a NUPAGE™ 4-12% Bis-Tris gel and stained with the standard protocol using 0.2 μM of the stain in 20 mM phosphate buffer pH 7.8. The Typhoon 8600 from Amersham Biosciences was used to scan the image using the 532 nm laser and the 580 long pass filter which allows light longer than 580 nm to the be detected by the photo-multiplier tube. A normal scan was performed and the following gel image (FIG. 5) was obtained after a single pass. As estimated visually, under these conditions, the sensitivity appears to be less than the indicated 8 nanograms and would be expected to be even greater if the multiple scanning option of the scanner was used.

FIG. 5. Sensitivity of Compound 19 in NUPAGE™ Gels Using the Typhoon Laser Based Scanner
Lane 1: E. coli lysate plus 1.0 ng of BenchMark™ 60 kDa protein
Lane 2: E. coli lysate plus 2.0 ng of BenchMark™ 60 kDa protein
Lane 3: E. coli lysate plus 4.0 ng of BenchMark™ 60 kDa protein
Lane 4: E. coli lysate plus 8.0 ng of BenchMark™ 60 kDa protein
Lane 5: E. coli lysate plus 16 ng of BenchMark™ 60 kDa protein
Lane 6: E. coli lysate plus 32 ng of BenchMark™ 60 kDa protein
Lane 7: E. coli lysate plus 64 ng of BenchMark™ 60 kDa protein
Lane 8: E. coli lysate plus 128 ng of BenchMark™ 60 kDa protein
Lane 9: E. coli lysate plus 256 ng of BenchMark™ 60 kDa protein
Lane 10: BenchMark™ 10 protein standard 5 μL load

### Example 27

### Microwave-Assisted Gel Staining

Although the new stain protocol is less cumbersome than traditional silver staining, it can be improved by having a shorter protocol for staining, such as a protocol assisted by microwave heating. The Invitrogen SilverQuest™ kit utilizes microwave-assisted heating to accelerate the time of staining from around 2 hours to just over 30 minutes. In the following procedure, the overall staining time is about 75 minutes. The microwave-assisted staining protocol is as follows:

TABLE 3
Microwave Assisted Staining Protocol used
for NuPAGE ™ Bis-Tris gels

| Step | Solution | Amount | Time |
|---|---|---|---|
| Fix | 10% Acetic acid/40% | 100 mL | Microwave 30 sec |
| | ethanol in water | | Then mix 10 min |
| Wash | Ultrapure water | 100 mL | Microwave 30 sec |
| | | | Then mix 5 min |
| Wash | Ultrapure water | 100 mL | Microwave 30 sec |
| | | | Then mix 5 min |
| Stain | 0.2 M Compound 17a | 50 mL | Mix 40 min |
| | in 20 mM | | |
| | Phopshate pH 7.8 | | |
| Wash | 20 mM Phosphate | 100 mL | Microwave 30 sec |
| | pH 7.8 | | Then mix 5 min |
| | Additonal washes may | Total | ~70 min |
| | be performed to reduce | Time: | |
| | background using 20 mM | | |
| | Phosphate pH 7.8 | | |

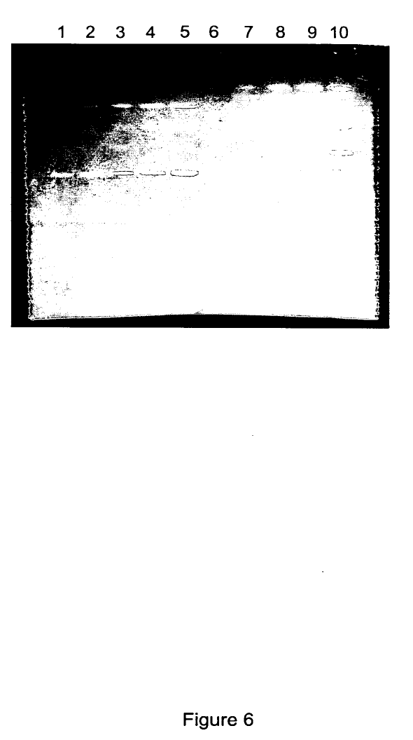

FIG. 6 is a picture of a NuPAGE™ 4-12% Bis-Tris gel stained using this protocol.

FIG. 6. Microwave Assisted InVision his-Tag Staining of a NUPAGE™ Bis-Tris Gel.
Lanes 1-5: Two-Fold dilutions of the LMW Markers from Amersham Biosciences.
Lanes 6-10: Two-fold dilutions of the BenchMark™ His-Tagged Protein Ladder.

### Example 28

### Purification of His-Tagged Proteins

The staining reagents of the invention can also be used in methods to purify His-tagged proteins or other affinity tagged proteins. In this embodiment, a protein to be purified is bound to a 6× His tag or other affinity tag to form a protein-affinity tag composition. The protein-affinity tag composition is then bound to the staining reagents described herein. The protein to be purified is purified using means known in the art.

His-tagged proteins or other affinity tagged proteins can be purified by affinity chromatography, in which compounds that bind His-tagged proteins (i.e., ligands, e.g., Ni⁺⁺) are attached to a solid support by a linker. A spacer may also be included between the solid support and the ligand, and can be placed on either side of the linker.

One specific technique for purifying His-tagged proteins or other affinity tagged proteins is known generally as Immobilized Metal Affinity Chromatography (IMAC). The technique derives from the discovery of proteins that have an affinity for heavy metal ions. For example, proteins containing certain sequences having histidine or cysteine residues have been found to complex with chelated zinc, nickel, cobalt or copper ions and become adsorbed on a chelating resin. See, for example, Porath et al., Metal Chelate Affinity Chromatography, A New Approach To Protein Fractionation, Nature 258:598-599 (1975); Hubert et al., Metal Chelate Affinity Chromatography, J. Chromatography 198:247-255 (1980); Kato et al., High Performance Metal Chelate Affinity Chromatography of Proteins, J. Chromatography 354:511-517 (1986); Fanou-Ayi et al., Metal-Chelate Affinity Chromatography as a Separation Tool, Annals New York Academy of Sciences 413:300-306 (1983); Fatiadi et al., Affinity Chromatography and Metal Chelate Affinity Chromatography, CRC Reviews in Analytical Chemistry 18:1-44. (1987); Hochuli et al., New Metal Chelate Adsorbent Selective for Proteins and Peptides Containing Neighboring Histidine Residues, J. Chromatography, 411, pp. 177-184 (1987). A comprehensive review is Wong, S H (1991) Chemistry of Protein Conjugation and Cross-linking, CRC Press, Boca Raton, Fla.

Affinity tags are typically peptides having a sequence capable of being specifically bound to and eluted from one or more ligands of an affinity matrix such as a chromatography support or bead. Typical examples of an affinity tag include an epitope, which can bind to a matrix-immobilized antibody, or a specific binding protein. Preferred affinity tags are those which are elutable from the affinity matrix by mild conditions unlikely to disrupt the protein to be purified and/or elute nonspecifically associated contaminants or that interact tightly with the affinity matrix such that specific elution conditions can be employed to preferentially elute the interacting proteins but retain some or all of the protein to be purified. Exemplified herein is a 6×-His tag, which is known to specifically bind to a column of nickel (Ni²⁺) or cobalt (Co²⁺) with high affinity (Crowe et al., In Methods in Molecular Biology, Harwood, A. J., eds., Vol. 31:371-387, Humana Press, Inc. Otawa, 1994; Porath et al., J. Protein Expr. Purif. 3:263-281, 1992. Another example of an affinity tag is a 12 amino acid peptide, known as the Protein C tag in the art, which is recognized in a calcium dependent manner by the commercially available monoclonal antibody HPC4 (Roche Applied Science, Indianapolis, Ind.). When greater purity is desired, sequential affinity purification steps can be used. Alternative affinity purification steps can allow for customization of the purification (e.g. a protein to be purified may bind to one affinity tag but not another when more than one affinity tag is used).

Affinity chromatography solid supports may include but are not limited to glass, agarose, polyacrylamine, dextran including crosslinked dextran (e.g., Sepharose™), cellulose, and substituted cellulose such as carboxymethylcellulose and cellulose carbonate, alumina, hydroxyalkylmethacrylate or mixtures thereof. Typically, the support initially comprises a reactive moiety, such as hydroxyl, carboxyl, amine, phenol, anhydride, aldehyde, epoxide or thiol, that is free to react with compounds such as spacers or linkers.

Bifunctional linkers, molecules that comprise two reactive groups, which may be different (heterobifunctional) or the same (homobifunctional), are preferred. Many of these are known in the art and include, by way of non-limiting example, N-succinimidyl-3-(2-pyridyldithio)propinate (SPDP), which activates and allows formation of a bridge between two sulfhydryl groups of cysteines or a bridge between a derivatized (propinated-thiolyated) primary amino group and a cysteine; m-maleimidobenzoyl-N-hydroxy-succimide ester (MBS), which activates an amino group and then couples by a sulfhydryl group to a cysteine sulfydryl so as to form a disulfide bond between the two polypeptides; and 1-ethyl-3-(3-dimethylaminopropyl)carbodiimide (EDC), which can cross-link two polypeptides by sequentially activating the carboxyl group of one polypeptide and then adding such to an amino group of another polypeptide. N-isocyano-ethylmorphlin, bis-diazotized-benzidine, benzoquone and glutaraldehyde, which are other reagents commonly employed to link polypeptides, can be employed in the present invention and are available from Pierce Chemical, Rockford, Ill.; Eastman Kodak Chemicals, Rochester, N.Y.; Serva, Westbury, N.Y.; Sigma Chemical Co., St. Louis, Mo.; and E. Merck, Damstadt, West Germany, for example. See, for example, Briand et al, Synthetic Peptides as Antigens: Pitfalls of Conjugation Methods, J. Immunol. Meth. 78:59-69 (1985); Kitagawa et al., Enzyme Coupled Immunoassay of Insulin Using a Novel Coupling reagent, J. Biochem. (Tokyo) 79:233-236 (1976); Ternynck et al., Conjugation of p-Benzoquinone Treated Enzymes with Antibodies and Fab Fragments, Immunochem. 14:767-774 (1977); and Drevin et al., Covalent Coupling of Proteins to Erythrocytes by Isocyanide. A New, Sensitive and Mild Technique for Identification and Estimation of Antibodies by Passive Hemagglutination, J. Immunol. Meth. 77:9-14 (1985).

Spacers may include, but are not limited to, p-benzoquinone, bis-(diazobenzidine), 3,6-bis-(mercurimethyl) dioxane, bisoxiranes, cyanuric chloride, p,p′-difluoro-m,m′-, dicyclohexylcarbodiimide, dinitrophenylsulphone, dimethyladipimidate, dimethylsuberimidate, divinylsulphone, N,N′-ethylene-bis-(iodoacetamide), glutaraldehye, hexamethylene bis-(male-imide), hexamethylene diisocyanate, N,N′-1,3-phenylene-bis-(maleimide), phenol-2,4-disulphonyl chloride, tetra-azotised o-dianisidine, toluene diisocyanate, Woodward's K reagent, water soluble carbodiimides, 6-aminohexanoic acid, hexamethylenedi-amine, 1,7-diamino-4-aza-heptane (3,3′-diamino-dipropylamine), and aminoacids or peptides.

Many solid supports, linkers and spacers are known in the art, as are methods by which to directly or indirectly attach compounds (e.g., a compound of the invention, such as a compound of Formula I) to such solid supports. In brief, linkers, preferably bifunctional linkers are chemically reacted with, in either order or simultaneously, a reactive moiety on the solid support or a compound of Formula I. Optionally, a spacer is also introduced through a chemical reaction or reactions. See, for example, Affinity Chromatography, A Practical Approach, IRL Press, Ltd., Oxford England (1985).

### Example 29

### Synthesis of Compound 20

2-Benzoxazole was alkylated at the ring nitrogen with 3-iodopropanoic acid to give the quaternary ammonium salt by refluxing in dichlorobenzene with sodium iodide at 130° C. Two equivalents of the quaternary ammonium salt were reacted with one equivalent of CH(OEt)₃ with refluxing in pyridine to afford a dimer. The dimer was reacted with NHS and DCC in acetonitrile/DMF to prepare a di-N-hydroxysuccinimide ester. The ester was reacted with N,N-Bis(carboxymethyl)lysine to prepare Compound 20.

Compound 20 was loaded with nickel by addition of nickel sulfate. The loaded bis-chelate was purified by C-18 chromatography using a Waters SEP-PAK cartridge following the manufacturer's suggested protocol.

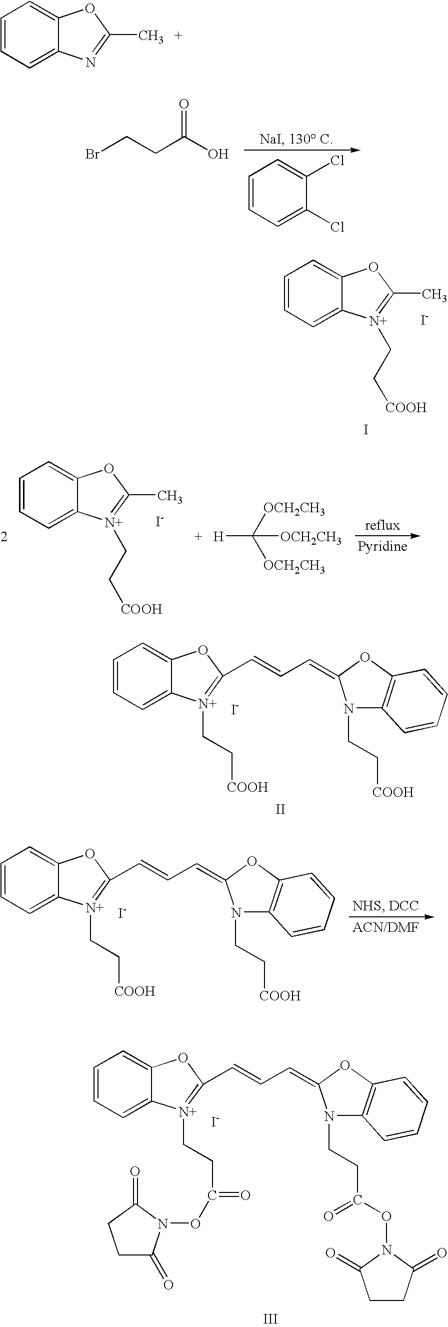

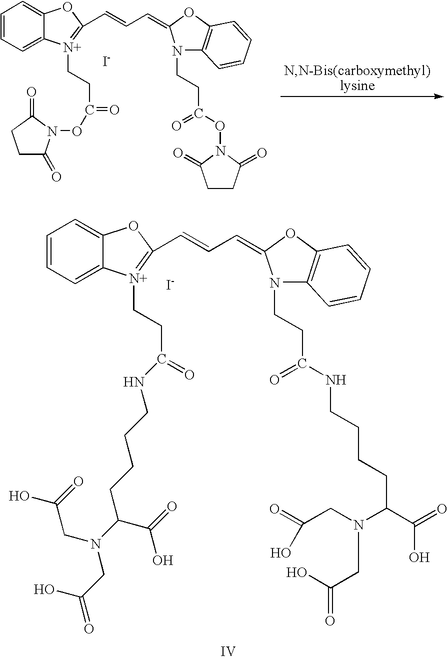

The protein staining sensitivity of t Compound 20 was compared against the sulfonated Cy2 dye (Amersham Pharmacia; Piscataway, N.J.). Both dyes exhibited similar sensitivity.

The preceding examples can be repeated with similar success by substituting the specifically described fluorescent compound, affinity tag and staining conditions of the preceding examples with those generically and specifically described in the forgoing description. One skilled in the art can easily ascertain the essential characteristics of the present invention, and without departing from the spirit and scope thereof, can make various changes and modifications of the invention to adapt to various usages and conditions.

All patents and patent applications mentioned in this specification are herein incorporated by reference to the same extent as if each individual publication, patent or patent application was specifically and individually indicated to be incorporated by reference.

What is claimed is:

2. A staining solution comprising:
a) a fluorescent compound having formula A(L)m(B)n wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH of about 7.0 to about 9.0 with the proviso that the binding domain does not comprise an antibody or fragment thereof.

3. The staining solution according to claim 1, wherein the fluorescent compound comprises a metal ion.

4. The staining solution according to claim 2, wherein the metal ion is nickel or cobalt.

5. The staining solution according to claim 2, wherein the fluorescent compound is

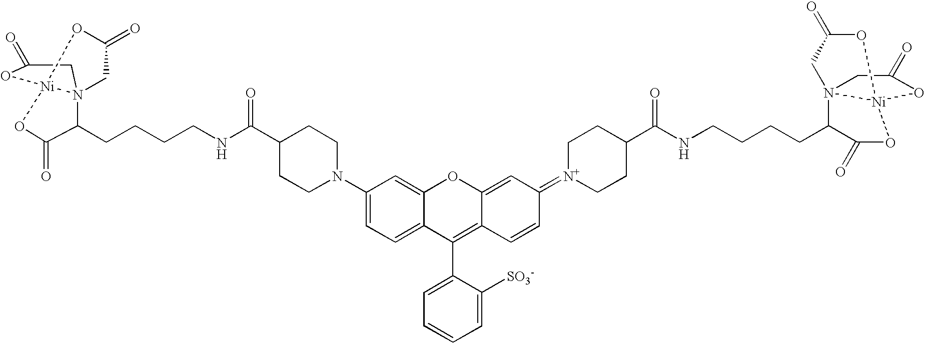

6. The staining solution according to claim 1, wherein the buffer comprises a salt.

7. The staining solution according to claim 1, wherein the buffer has a pH of about 7.8.

8. The staining solution according to claim 1, wherein the buffer comprises aqueous phosphate.

9. The staining solution according to claim 7, wherein the phosphate is present at about 20 mM.

10. The staining solution according to claim 1, wherein the fluorophore is xanthene, coumarin, cyanine, acridine, anthracene, benzofuran, indole or borapolyazaindacene.

11. The staining solution according to claim 1, wherein the binding domain is NTA or BAPTA.

12. A staining solution comprising
a) a fluorescent compound comprising nickel ions, wherein the fluorescent compound has the formula A(L)m(B)n wherein A is a fluorophore that is xanthene, coumarin, cyanine, acridine, anthracene, benzofuran, indole or borapolyazaindacene, L is a linker, B is an acetic acid binding domain that is NTA or BAPTA, m is an integer from 1 to 4 and n is an integer from 1 to 6; and,
b) a buffer having a pH of about 7.0 to about 9.0 comprising about 20 mM phosphate;
with the proviso that the binding domain does not comprise an antibody or fragment thereof.

13. The staining solution according to claim 11, wherein the fluorescent compound is

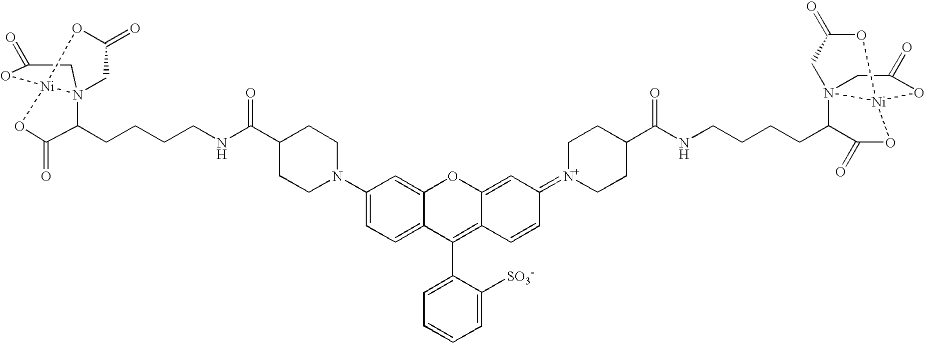

14. The staining solution according to claim 11, wherein the pH is about 7.8.

15. A method for detecting the presence or absence of an affinity tag containing fusion protein in a sample, the method comprising:
a) contacting the sample with a staining solution according to any one of claims 1-13 to prepare a contacted sample;
b) illuminating the fluorescent compound with a suitable light source to prepare an illuminated sample; and,
c) observing the illuminated sample whereby the presence or absence of the fusion protein is detected.

16. The method according to claim 14, wherein the method further comprises immobilizing the sample on a solid or semi-solid matrix prior to the contacting step.

17. The method according to claim 14, wherein the affinity tag is a poly-histidine.

18. A method for detecting a poly-histidine affinity tag containing fusion protein in a sample, the method comprising:
i) immobilizing the sample on a solid or semi-solid matrix to prepare an immobilized sample;
ii) contacting the immobilized sample with a staining solution to prepare a stained sample, wherein the staining solution comprises
a) a fluorescent compound having formula A(L)m(B)n wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH of about 7.0 to about 9.0;
iii) incubating the stained sample for a sufficient amount of time to allow the fluorescent compound to associate with the poly-histidine affinity tag to prepare an incubated sample;
iv) illuminating the incubated sample with a suitable light source to prepare an illuminated sample; and
v) observing the illuminated sample whereby the fusion protein is detected.

19. The method according to claim 17, wherein the buffer has a pH of about 7.8.

20. The method according to claim 17, wherein the fluorophore is xanthene, cyanine, coumarin, acridine, anthracene, benzofuran, borapolyazaindacene or a derivative thereof.

21. The method according to claim 17, wherein fluorescent compound of the staining solution comprises at least three acetic acid groups.

22. The method according to claim 17, wherein the acetic acid groups are complexed with nickel ions or cobalt ions.

23. The method according to claim 17, wherein immobilizing the sample comprises electrophoretically separating on a polymeric gel.

24. The method according to claim 22, further comprising adding a fixing solution to the immobilized sample.

25. The method according to claim 23, wherein the fixing solution comprises an alcohol.

26. The method according to claim 22, further comprising contacting the gel with a total protein stain after the presence or absence of the fusion protein is detected.

27. The method according to claim 17, wherein the fluorescent compound is

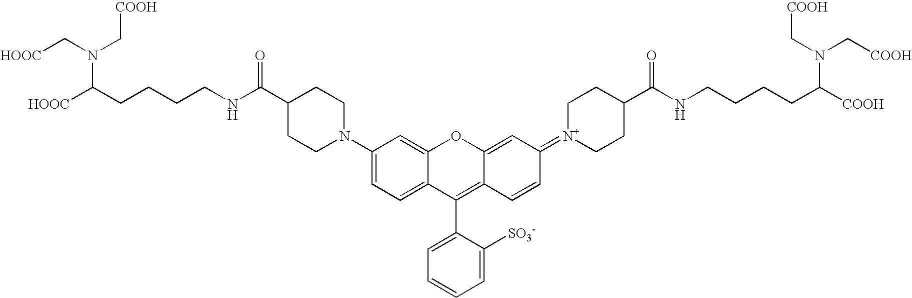

28. The method according to claim 26, wherein the fluorescent compound is complexed with a nickel ion or a cobalt ion.

29. The method according to claim 27, wherein the fluorescent compound is

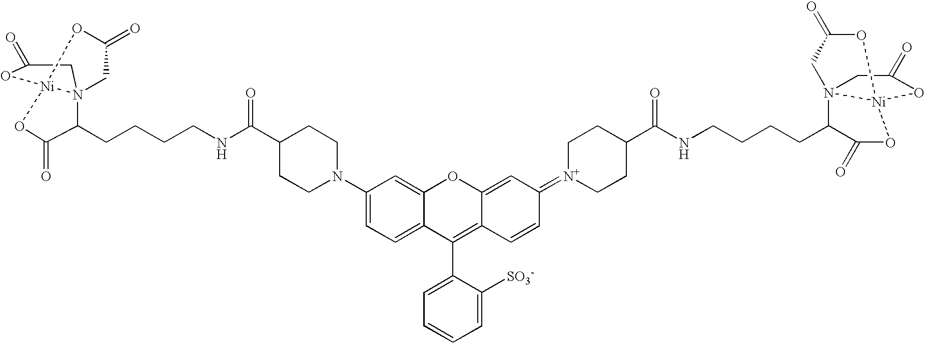

30. The method according to claim 17, wherein the fluorescent compound is 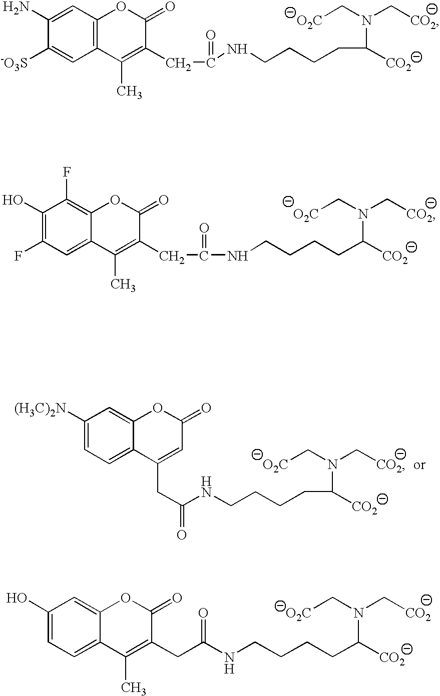
and salts thereof.

31. The method according to claim 29, wherein the fluorescent compound is complexed with nickel ions or cobalt ions.

32. The method according to claim 17, wherein the fluorescent compound is 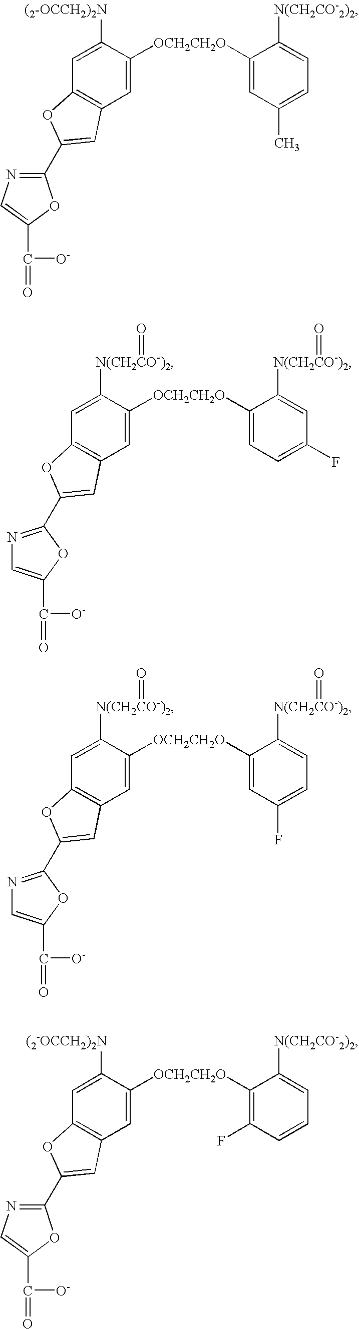 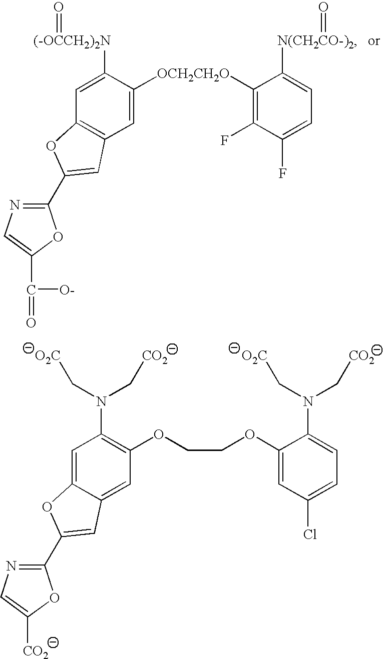
and salts thereof.

33. The method according to claim 31, wherein the fluorescent compound is complexed with nickel ions or cobalt ions.

34. The method according to claim 17, wherein the fluorescent compound is 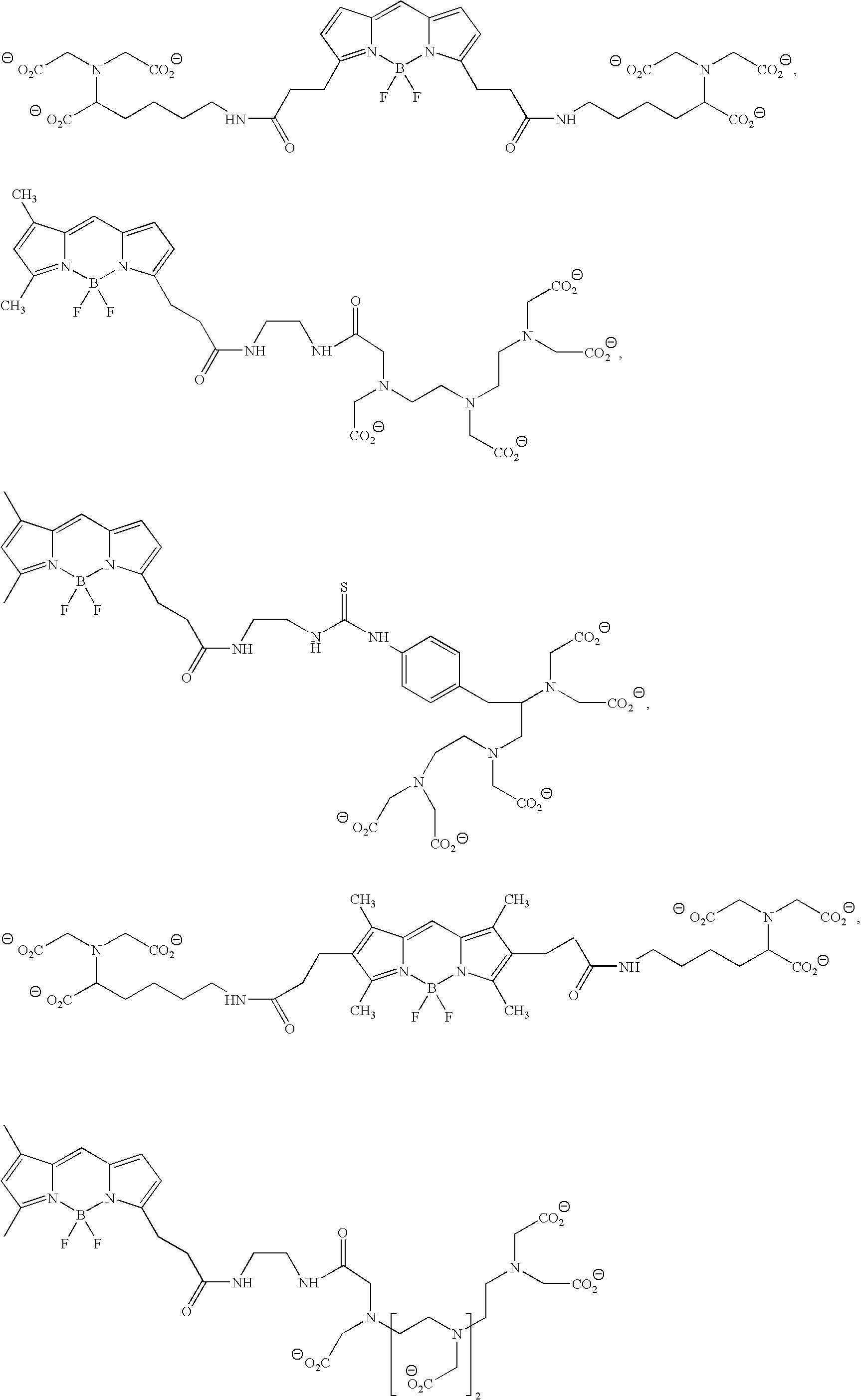 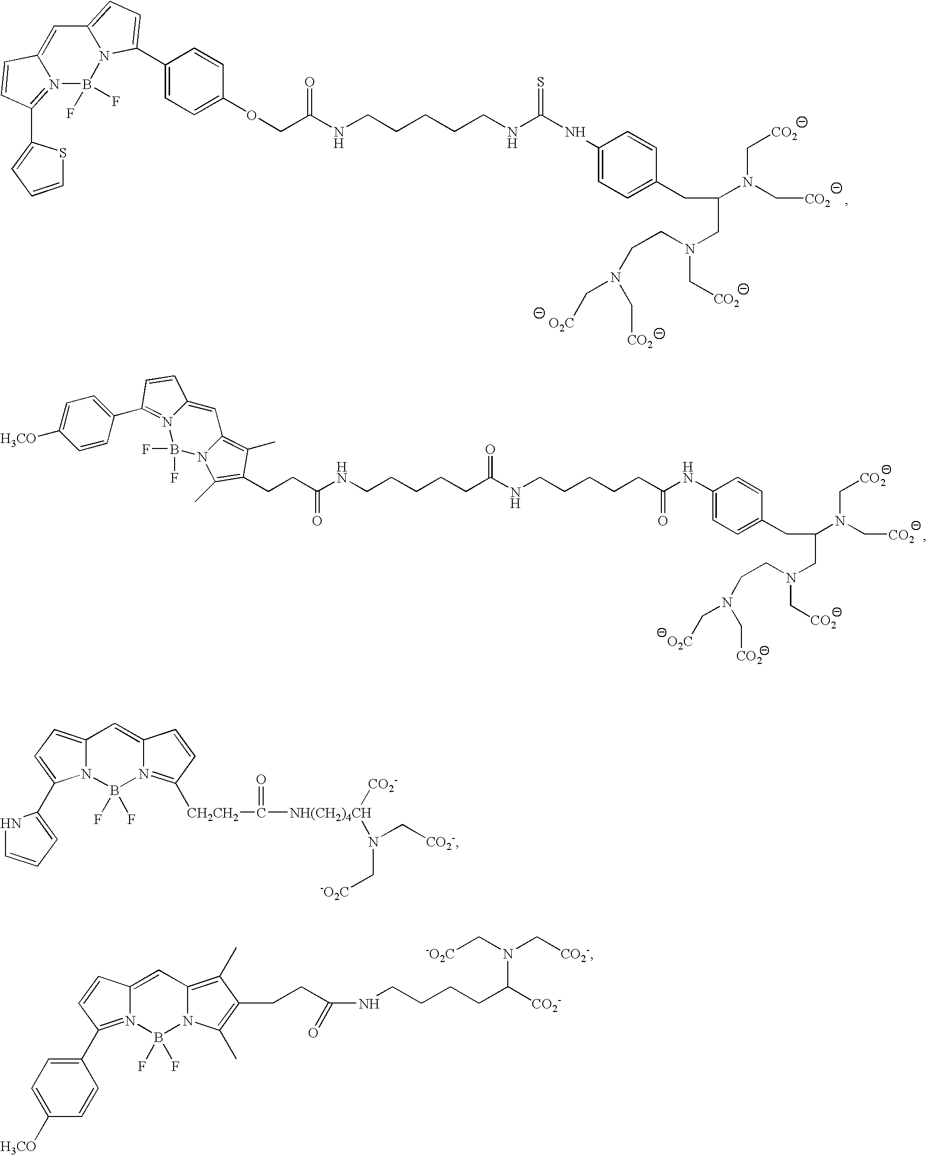 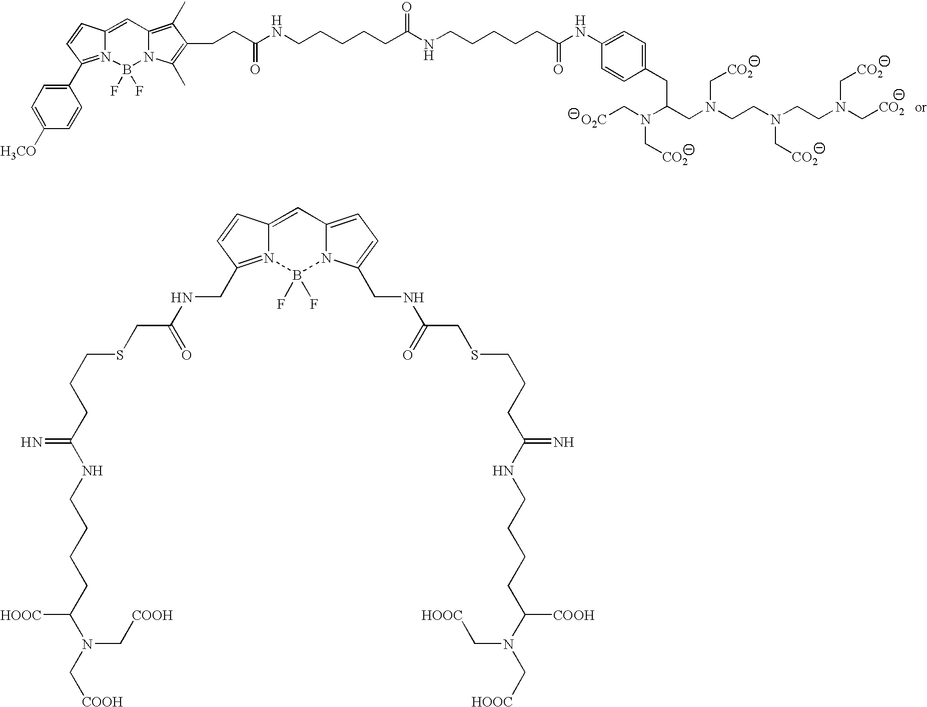
and salts thereof.

35. The method according to claim 33, wherein the fluorescent compound is complexed with nickel ions or cobalt ions.

36. A kit for detecting an affinity tag containing fusion protein, wherein the kit comprises; a staining solution comprising:
a) a fluorescent compound having formula A(L)m(B)n wherein A is a fluorophore, L is a linker, B is an acetic acid binding domain capable of selectively binding to a poly-histidine affinity tag, m is an integer from 1 to 4 and n is an integer from 1 to 6; and,
b) a buffer having a pH of about 7.0 to about 9.0;
with the proviso that the binding domain does not comprise an antibody or fragment thereof.

37. The kit according to claim 35, further comprising a molecular weight markers, a fixing solution, a wash solution or an additional detection reagent.

38. The kit according to claim 36, wherein the additional detection reagent is a total protein stain.

39. The kit according to claim 35, wherein the fluorescent compound comprises a binding domain and a fluorophore selected from the group consisting of a xanthene, cyanine, coumarin, acridine, anthracene, benzofuran, borapolyazaindacene and derivative thereof.

40. The kit according to claim 35, wherein the binding domain is NTA or BAPTA.

41. The kit according to claim 35, wherein the fluorescent compound is complexed to nickel ions or cobalt ions.

42. The kit according to claim 35, wherein the fluorescent compound is

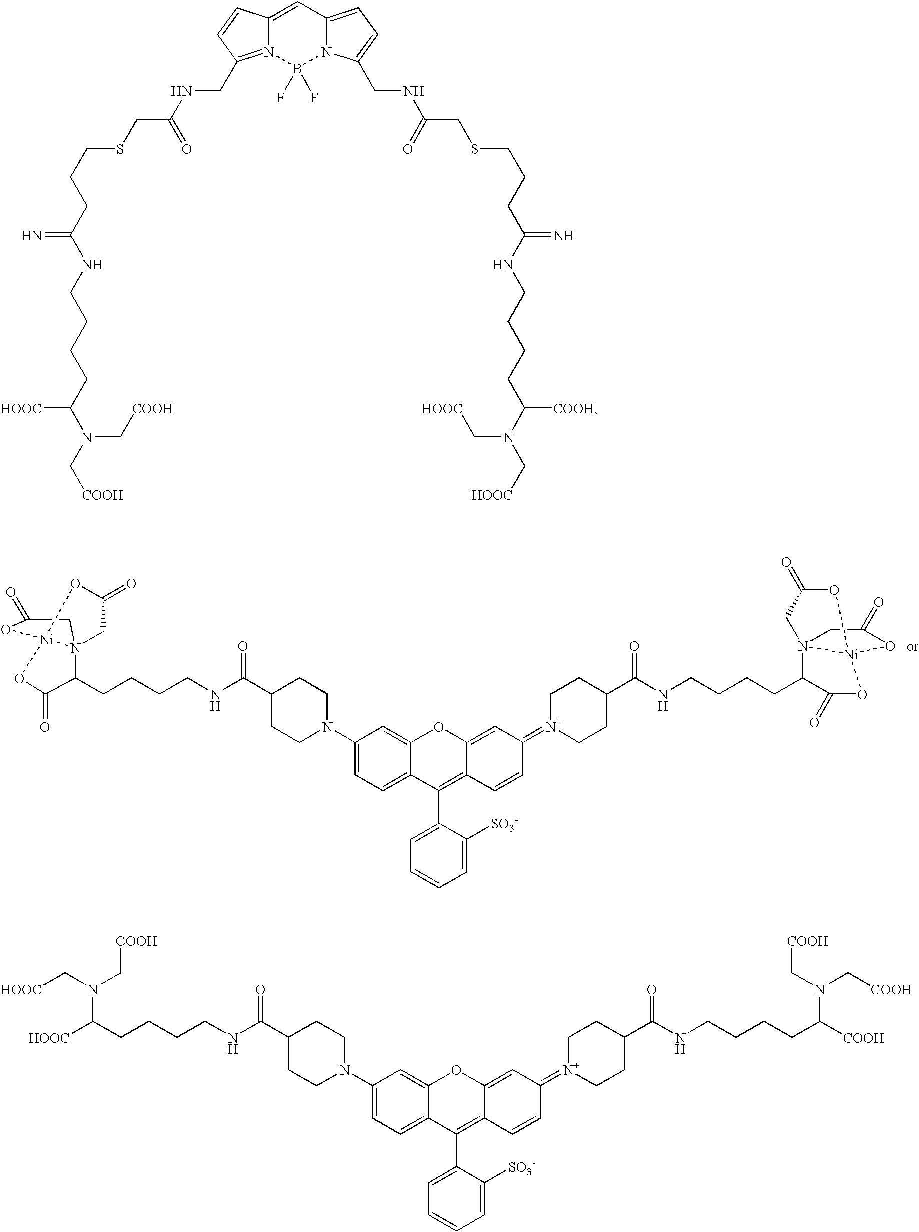

42. A fluorescent compound having the chemical structure: [chemical structure]
